(19) United States
(12) Patent Application Publication
(10) Pub. No.: US 2013/0066088 A1
(43) Pub. Date: Mar. 14, 2013

(54) METHODS FOR OBTAINING HIGH-YIELDING OIL PALM PLANTS

(71) Applicants: Tony Ooi Eng Keong, Selangor Darul Ehsan (MY); Leona Daniela Jeffery Daim, Selangor Darul Ehsan (MY); Yeap Wan Chin, Selangor Darul Ehsan (MY); Ng Boon Zean, Selangor Darul Ehsan (MY); Lee Fong Chin, Selangor Darul Ehsan (MY); Ainul Masni bt Othman, Selangor Darul Ehsan (MY); Harikrishna a/l Kulaveerasingam, Selangor Darul Ehsan (MY); Mohd. Nazir Basiran, Selangor Darul Ehsan (MY); Mohaimi Mohamed, Selangor Darul Ehsan (MY)

(72) Inventors: Tony Ooi Eng Keong, Selangor Darul Ehsan (MY); Leona Daniela Jeffery Daim, Selangor Darul Ehsan (MY); Yeap Wan Chin, Selangor Darul Ehsan (MY); Ng Boon Zean, Selangor Darul Ehsan (MY); Lee Fong Chin, Selangor Darul Ehsan (MY); Ainul Masni bt Othman, Selangor Darul Ehsan (MY); Harikrishna a/l Kulaveerasingam, Selangor Darul Ehsan (MY); Mohd. Nazir Basiran, Selangor Darul Ehsan (MY); Mohaimi Mohamed, Selangor Darul Ehsan (MY)

(21) Appl. No.: 13/362,535

(22) Filed: Jan. 31, 2012

(30) Foreign Application Priority Data
Sep. 13, 2011 (MY) PI 2011004343

(51) Int. Cl. C11B 1/00; G01N 33/566; G01N 33/573; A01H 5/00

(52) U.S. Cl. 554/8; 800/298; 436/501; 435/7.4

(57) ABSTRACT

Methods are provided for obtaining a high-yielding oil palm plant, comprising determining the level of a protein in mesocarp tissue of a fruit of a parental oil palm plant, determining whether there is a difference between the level of the protein in the mesocarp tissue of the fruit of the parental oil palm plant and the level of the protein in mesocarp tissue of a fruit of a reference oil palm plant, and selecting progeny of the parental oil palm plant based on the difference to obtain the high-yielding oil palm plant. Also provided are methods for predicting oil yield of a test oil palm plant and kits for obtaining a high-yielding oil palm plant.

### TECHNICAL FIELD

This application relates to methods for obtaining high-yielding plants, and more particularly to methods for obtaining oil palm plants that are high-yielding with respect to producing palm oil.

### BACKGROUND ART

The African oil palm Elaeis guineensis Jacq. is an important oil-food crop. Oil palm plants are monoecious, i.e. single plants produce both male and female flowers, and are characterized by alternating series of male and female inflorescences. The male inflorescence is made up of numerous spikelets, and can bear well over 100,000 flowers. Oil palm is naturally cross-pollinated by insects and wind. The female inflorescence is a spadix which contains several thousands of flowers borne on thorny spikelets. A bunch carries 500 to 4,000 fruits. The oil palm fruit is a sessile drupe that is spherical to ovoid or elongated in shape and is composed of an exocarp, a mesocarp containing palm oil, and an endocarp surrounding a kernel.

Oil palm is important both because of its high yield and because of the high quality of its oil. Regarding yield, oil palm is the highest yielding oil-food crop, with a recent average yield of 3.67 tonnes per hectare per year and with best progenies known to produce about 10 tonnes per hectare per year. Oil palm is also the most efficient plant known for harnessing the energy of sunlight for producing oil. Regarding quality, oil palm is cultivated for both palm oil, which is produced in the mesocarp, and palm kernel oil, which is produced in the kernel. Palm oil in particular is a balanced oil, having almost equal proportions of saturated fatty acids (≈55% including 45% of palmitic acid) and unsaturated fatty acids (≈45%), and it includes beta carotene. The palm kernel oil is more saturated than the mesocarp oil. Both are low in free fatty acids. The current combined output of palm oil and palm kernel oil is about 50 million tonnes per year, and demand is expected to increase substantially in the future with increasing global population and per capita consumption of oils and fats.

Although oil palm is the highest yielding oil-food crop, current oil palm crops produce well below their theoretical maximum. Moreover, conventional methods for identifying potential high-yielding palms for use in crosses to generate progeny with higher yields require cultivation of palms and measurement of production of oil thereby over the course of many years, which is both time and labor intensive. In addition, conventional breeding techniques for propagation of oil palm for oil production are also time and labor intensive, particularly because the most productive, and thus commercially relevant, palms exhibit a hybrid phenotype which makes propagation thereof by direct hybrid crosses impractical. Accordingly, a need exists to improve oil palm yields through improved methods for obtaining and identifying high-yielding palms.

Transgenic approaches offer potential solutions to the general problem of the need to increase plant yields. For example, transgenic modification of crops such as soy and corn by the introduction of pest resistance genes derived from other organisms is now well known as a means for increasing crop yields. Moreover, methods for increasing plant yields by increasing or generating in the plant activities of particular proteins have also been disclosed, for example by Schön et al., WO 2010/046221. However, transgenic modification of crops raises potential concerns regarding unintended detrimental effects on individuals and ecosystems.

Proteomics, which encompasses the study of the protein complement of a genome, also offers potential solutions to the general problem of increasing plant yields. For example, difference gel electrophoresis (“DIGE”) analysis, corresponding to two dimensional gel electrophoresis employing sensitive fluorescent labeling dyes, as described by Mackintosh et al., 3 Proteomics 2273-88 (2003), has been successfully employed in protein expression analyses in rice and sunflower, as described by Teshima et al., Regulatory Toxicology & Pharmacology (article in press), and Hajduch et al., 6 Journal of Proteome Research 3232-41 (2007), respectively. In rice, this approach was used to differentiate one cultivar from others, and also to compare expression of allergen proteins. In sunflower, several leads in seed oil traits have been identified for further investigation. However, given the many differences in the genetics and metabolism of rice, sunflower, and oil palm, and the highly specific nature of protein expression, these studies in rice and sunflower would not be expected to be useful with respect to improving oil palm yields through improved methods for obtaining and identifying high-yielding palms.

### DISCLOSURE OF INVENTION

A method is provided for obtaining a high-yielding oil palm plant. The method comprises determining the level of a protein in mesocarp tissue of a fruit of a parental oil palm plant. The protein is selected from the group consisting of 5-methyltetrahydropteroyltriglutamate-homocysteine methyltransferase, abscisic stress ripening protein, actin 6, actin E, biotin carboxylase precursor, caffeic acid O-methyltransferase, catalase 2, conserved-hypothetical-protein-of-Ricinus-communis ortholog, fibrillin-like protein, flavodoxin-like quinone reductase 1, fructose-bisphosphate aldolase, glyceraldehyde 3-phosphate dehydrogenase, H0825G02.11 ortholog, large subunit of ribulose-1,5-bisphosphate carboxylase/oxygenase, LealP, methionine synthase protein, mitochondrial peroxiredoxin, Os02g0753300 ortholog, Os05g0482700 ortholog, Os12g0163700 ortholog, OSJNBb0085F13.17 ortholog, predicted-protein-of-Ostreococcus-lucimarinus-CCE9901 ortholog, predicted-protein-of-Physcomitrella patens-subsp.-patens ortholog, predicted-protein-of-Populus-trichocarpa ortholog, hypothetical-protein-isoform-1-of-Vitis-vinifera ortholog, nascent polypeptide associated complex alpha, proline iminopeptidase, protein transporter, putative-NBS-LRR-disease-resistance-protein-homologue-of-Oryza-sativa-Japonica-Group ortholog, Ran GTPase binding protein, chloroplastic triosephosphate isomerase, V-type proton ATPase catalytic subunit A, regulator of ribonuclease activity A, retroelement pol polyprotein-like ortholog, ribosomal protein L10, short chain type dehydrogenase, temperature-induced lipocalin, and unknown-protein-of-Picea-sitchensis ortholog. The method also comprises determining whether there is a difference between the level of the protein in the mesocarp tissue of the fruit of the parental oil palm plant and the level of the protein in mesocarp tissue of a fruit of a reference oil palm plant. The method also comprises selecting progeny of the parental oil palm plant based on the difference to obtain the high-yielding oil palm plant.

Also provided is a method for predicting oil yield of a test oil palm plant. The method comprises determining the level of a protein in mesocarp tissue of a fruit of the test oil palm plant. The protein is selected from the group consisting of 5-methyltetrahydropteroyltriglutamate-homocysteine methyltransferase, abscisic stress ripening protein, actin 6, actin E, biotin carboxylase precursor, caffeic acid O-methyltransferase, catalase 2, conserved-hypothetical-protein-of-Ricinus-communis ortholog, fibrillin-like protein, flavodoxin-like quinone reductase 1, fructose-bisphosphate aldolase, glyceraldehyde 3-phosphate dehydrogenase, H0825G02.11 ortholog, large subunit of ribulose-1,5-bisphosphate carboxylase/oxygenase, LealP, methionine synthase protein, mitochondrial peroxiredoxin, Os02g0753300 ortholog, Os05g0482700 ortholog, Os12g0163700 ortholog, OSJNBb0085F13.17 ortholog, predicted-protein-of-Ostreococcus-lucimarinus-CCE9901 ortholog, predicted-protein-of-Physcomitrella patens-subsp.-patens ortholog, predicted-protein-of-Populus-trichocarpa ortholog, hypothetical-protein-isoform-1-of-Vitis-vinifera ortholog, nascent polypeptide associated complex alpha, proline iminopeptidase, protein transporter, putative-NBS-LRR-disease-resistance-protein-homologue-of-Oryza-sativa-Japonica-Group ortholog, Ran GTPase binding protein, chloroplastic triosephosphate isomerase, V-type proton ATPase catalytic subunit A, regulator of ribonuclease activity A, retroelement pol polyprotein-like ortholog, ribosomal protein L10, short chain type dehydrogenase, temperature-induced lipocalin, and unknown-protein-of-Picea-sitchensis ortholog. The method also includes determining whether there is a difference between the level of the protein in the mesocarp tissue of the fruit of the test oil palm plant and the level of the protein in mesocarp tissue of a fruit of a reference oil palm plant. The method also includes predicting the oil yield of the test oil palm plant based on the difference.

Also provided is a kit for obtaining a high-yielding oil palm plant. The kit comprises an antibody for detection of a protein selected from the group consisting of 5-methyltetrahydropteroyltriglutamate-homocysteine methyltransferase, abscisic stress ripening protein, actin 6, actin E, biotin carboxylase precursor, caffeic acid O-methyltransferase, catalase 2, conserved-hypothetical-protein-of-Ricinus-communis ortholog, fibrillin-like protein, flavodoxin-like quinone reductase 1, fructose-bisphosphate aldolase, glyceraldehyde 3-phosphate dehydrogenase, H0825G02.11 ortholog, large subunit of ribulose-1,5-bisphosphate carboxylase/oxygenase, LealP, methionine synthase protein, mitochondrial peroxiredoxin, Os02g0753300 ortholog, Os05g0482700 ortholog, Os12g0163700 ortholog, OSJNBb0085F13.17 ortholog, predicted-protein-of-Ostreococcus-lucimarinus-CCE9901 ortholog, predicted-protein-of-Physcomitrella patens-subsp.-patens ortholog, predicted-protein-of-Populus-trichocarpa ortholog, hypothetical-protein-isoform-1-of-Vitis-vinifera ortholog, nascent polypeptide associated complex alpha, proline iminopeptidase, protein transporter, putative-NBS-LRR-disease-resistance-protein-homologue-of-Oryza-sativa-Japonica-Group ortholog, Ran GTPase binding protein, chloroplastic triosephosphate isomerase, V-type proton ATPase catalytic subunit A, regulator of ribonuclease activity A, retroelement pol polyprotein-like ortholog, ribosomal protein L10, short chain type dehydrogenase, temperature-induced lipocalin, and unknown-protein-of-Picea-sitchensis ortholog. The kit also comprises an extract of a mesocarp tissue of a fruit of a reference oil palm plant.

The disclosed methods and kits are based on an advantageous combination of proteomics, to identify markers for high and low-yielding traits in current oil palm breeding populations and thus to increase the pace of identification of high yielding palms, and conventional breeding techniques, to generate higher-yielding progeny therefrom. Applications include identifying high-yielding parental palm plants for use in generating higher-yielding progeny and predicting palm oil yields of test palms, in both cases without need for collecting oil yield data from palms over the course of years. Also, although the methods and kits are well suited for application to conventional breeding techniques, thus providing a basis for increasing the pace of obtaining high-yielding palms without relying on transgenics, the methods and kits can also be applied to improve the efficiency of propagation of oil palm by tissue culture or transgenic approaches too.

### BRIEF DESCRIPTION OF DRAWINGS

FIG. 1 is a scanned image of a two-dimensional fluorescence difference gel electrophoresis (“DIGE”) analytical gel corresponding to mesocarp protein of high-yielding palm H2 and low-yielding palm h1, both tested at 12 weeks post-pollination.

FIG. 2 is a scanned image of a DIGE analytical gel corresponding to mesocarp protein of high-yielding palm H4 and low-yielding palm h6, both tested at 12 weeks post-pollination.

FIG. 3 is a scanned image of a DIGE analytical gel corresponding to mesocarp protein of high-yielding palm H6 and low-yielding palm h9, both tested at 12 weeks post-pollination.

FIG. 4 is a scanned image of a DIGE analytical gel corresponding to mesocarp protein of high-yielding palm H2 and low-yielding palm h1, both tested at 16 weeks post-pollination.

FIG. 5 is a scanned image of a DIGE analytical gel corresponding to mesocarp protein of high-yielding palm H4 and low-yielding palm h6, both tested at 16 weeks post-pollination.

FIG. 6 is a scanned image of a DIGE analytical gel corresponding to mesocarp protein of high-yielding palm H6 and low-yielding palm h9, both tested at 16 weeks post-pollination.

FIG. 7 is a scanned image of a DIGE analytical gel corresponding to mesocarp protein of high-yielding palm H2 and low-yielding palm h1, both tested at 18 weeks post-pollination.

FIG. 8 is a scanned image of a DIGE analytical gel corresponding to mesocarp protein of high-yielding palm H4 and low-yielding palm h6, both tested at 18 weeks post-pollination.

FIG. 9 is a scanned image of a DIGE analytical gel corresponding to mesocarp protein of high-yielding palm H6 and low-yielding palm h9, both tested at 18 weeks post-pollination.

FIG. 10A-M is a list of sequences of the forty-five unique differentially expressed proteins identified herein, abbreviated in one-letter amino acid format.

### BEST MODE FOR CARRYING OUT THE INVENTION

The application is drawn to methods for obtaining high-yielding oil palm plants, methods for predicting oil yield of test oil palm plants, and kits for obtaining high-yielding oil palm plants. As disclosed herein, the level of a protein in mesocarp tissue of a fruit of an oil palm plant can be used for obtaining a high-yielding oil palm plant and for predicting oil yield of a test oil palm plant. Proteins useful in this regard include 5-methyltetrahydropteroyltriglutamate-homocysteine methyltransferase, abscisic stress ripening protein, actin 6, actin E, biotin carboxylase precursor, caffeic acid O-methyltransferase, catalase 2, conserved-hypothetical-protein-of-Ricinus-communis ortholog, fibrillin-like protein, flavodoxin-like quinone reductase 1, fructose-bisphosphate aldolase, glyceraldehyde 3-phosphate dehydrogenase, H0825G02.11 ortholog, large subunit of ribulose-1,5-bisphosphate carboxylase/oxygenase, LealP, methionine synthase protein, mitochondrial peroxiredoxin, Os02g0753300 ortholog, Os05g0482700 ortholog, Os12g0163700 ortholog, OSJNBb0085F13.17 ortholog, predicted-protein-of-Ostreococcus-lucimarinus-CCE9901 ortholog, predicted-protein-of-Physcomitrella patens-subsp.-patens ortholog, predicted-protein-of-Populus-trichocarpa ortholog, hypothetical-protein-isoform-1-of-Vitis-vinifera ortholog, nascent polypeptide associated complex alpha, proline iminopeptidase, protein transporter, putative-NBS-LRR-disease-resistance-protein-homologue-of-Oryza-sativa-Japonica-Group ortholog, Ran GTPase binding protein, chloroplastic triosephosphate isomerase, V-type proton ATPase catalytic subunit A, regulator of ribonuclease activity A, retroelement pol polyprotein-like ortholog, ribosomal protein L10, short chain type dehydrogenase, temperature-induced lipocalin, and unknown-protein-of-Picea-sitchensis ortholog. Accordingly, the application provides methods for obtaining high-yielding oil palm plants comprising determining the level of one of the above-noted proteins in mesocarp tissue of a fruit of a parental oil palm plant, determining whether there is a difference between the level of the protein in the mesocarp tissue of the fruit of the parental oil palm plant and the level of the protein in mesocarp tissue of a fruit of a reference oil palm plant, and selecting progeny of the parental oil palm plant based on the difference to obtain the high-yielding oil palm plant. Moreover, the application provides methods for predicting oil yield of test oil palm plants comprising determining the level of one of the above-noted proteins in mesocarp tissue of a fruit of the test oil palm plant, determining whether there is a difference between the level of the protein in the mesocarp tissue of the fruit of the test oil palm plant and the level of the protein in mesocarp tissue of a fruit of a reference oil palm plant, and predicting the oil yield of the test oil palm plant based on the difference. In addition, the application provides kits for obtaining high-yielding oil palm plants comprising an antibody for detection of one of the above-noted proteins and an extract of a mesocarp tissue of a fruit of a reference oil palm plant.

### DEFINITIONS

The term “parental oil palm plant,” as used herein, means an oil palm plant from which progeny have been generated, are generated, or will be generated during the course of carrying out methods for obtaining a high-yielding oil palm plant as disclosed herein or using kits for obtaining a high-yielding oil palm plant as disclosed herein.

The term “test oil palm plant,” as used herein, means an oil palm plant which has been subjected, is subjected, or will be subjected to a step of determining the level of a protein in mesocarp tissue of a fruit thereof during the course of carrying out methods for predicting oil yield of the plant as disclosed herein.

The term “reference oil palm plant,” as used herein, means an oil palm plant used as a basis for comparison in determining oil palm yield traits. The reference oil palm plant can be, for example, an oil palm plant that produces high, average, or low amounts of palm oil, depending on the context of the particular application. For example, the reference oil palm plant can be an oil palm plant that produces 10, 9, 8, 7, 6, 5, 4, 3, 2, or 1 tonnes of palm per hectare per year.

The terms “high-yielding,” “low-yielding,” and “oil yield,” as used herein with respect to the methods and kits disclosed herein, refer to yields of palm oil in mesocarp tissue of fruits of palm oil plants.

The term “homologs” and “homologous,” as used herein, refers to two or more genes having highly similar DNA sequences or two or more proteins having highly similar amino acid sequences. Such genes or proteins may be considered to be homologous based on sharing, for example, 60%, 70%, 75%, 80%, 85%, 90%, 91%, 92%, 93%, 94%, 95%, 96%, 97%, 98%, 99%, or greater sequence identity. The terms homologs and homologous encompass such highly similar genes or proteins, whether the genes or proteins are derived from a single species, and thus may represent structurally and functionally similar genes or proteins of the species, or from different species, and thus may represent orthologous genes or proteins derived from a common ancestor.

### Method for Obtaining a High-Yielding Oil Palm Plant

As noted above, a method is provided for obtaining a high-yielding oil palm plant. The method comprises: (i) determining the level of a protein in mesocarp tissue of a fruit of a parental oil palm plant; (ii) determining whether there is a difference between the level of the protein in the mesocarp tissue of the fruit of the parental oil palm plant and the level of the protein in mesocarp tissue of a fruit of a reference oil palm plant; and (iii) selecting progeny of the parental oil palm plant based on the difference to obtain the high-yielding oil palm plant.

### Proteins

In accordance with this method, the protein is selected from the group consisting of 5-methyltetrahydropteroyltriglutamate-homocysteine methyltransferase, abscisic stress ripening protein, actin 6, actin E, biotin carboxylase precursor, caffeic acid O-methyltransferase, catalase 2, conserved-hypothetical-protein-of-Ricinus-communis ortholog, fibrillin-like protein, flavodoxin-like quinone reductase 1, fructose-bisphosphate aldolase, glyceraldehyde 3-phosphate dehydrogenase, H0825G02.11 ortholog, large subunit of ribulose-1,5-bisphosphate carboxylase/oxygenase, LealP, methionine synthase protein, mitochondrial peroxiredoxin, Os02g0753300 ortholog, Os05g0482700 ortholog, Os12g0163700 ortholog, OSJNBb0085F13.17 ortholog, predicted-protein-of-Ostreococcus-lucimarinus-CCE9901 ortholog, predicted-protein-of-Physcomitrella patens-subsp.-patens ortholog, predicted-protein-of-Populus-trichocarpa ortholog, hypothetical-protein-isoform-1-of-Vitis-vinifera ortholog, nascent polypeptide associated complex alpha, proline iminopeptidase, protein transporter, putative-NBS-LRR-disease-resistance-protein-homologue-of-Oryza-sativa-Japonica-Group ortholog, Ran GTPase binding protein, chloroplastic triosephosphate isomerase, V-type proton ATPase catalytic subunit A, regulator of ribonuclease activity A, retroelement pol polyprotein-like ortholog, ribosomal protein L10, short chain type dehydrogenase, temperature-induced lipocalin, and unknown-protein-of-Picea-sitchensis ortholog.

In some embodiments, the 5-methyltetrahydropteroyltriglutamate-homocysteine methyltransferase comprises SEQ ID NO: 1, the abscisic stress ripening protein comprises SEQ ID NO: 2, the actin 6 comprises SEQ ID NO: 3, the actin E comprises SEQ ID NO: 4, the biotin carboxylase precursor comprises SEQ ID NO: 5, the caffeic acid O-methyltransferase comprises SEQ ID NO: 6, the catalase 2 comprises SEQ ID NO: 7, the conserved-hypothetical-protein-of-Ricinus-communis ortholog comprises SEQ ID NO: 8, the fibrillin-like protein comprises SEQ ID NO: 9, the flavodoxin-like quinone reductase 1 comprises SEQ ID NO: 10, the fructose-bisphosphate aldolase comprises SEQ ID NO: 11, the glyceraldehyde 3-phosphate dehydrogenase comprises SEQ ID NO: 12, the H0825G02.11 ortholog comprises SEQ ID NO: 13, the large subunit of ribulose-1,5-bisphosphate carboxylase/oxygenase comprises SEQ ID NO: 14, the LealP comprises SEQ ID NO: 15, the methionine synthase protein comprises SEQ ID NO: 16, the mitochondrial peroxiredoxin comprises SEQ ID NO: 17, the Os02g0753300 ortholog comprises SEQ ID NO: 18, the Os05g0482700 ortholog comprises SEQ ID NO: 19, the Os12g0163700 ortholog comprises SEQ ID NO: 20, the OSJNBb0085F13.17 ortholog comprises SEQ ID NO: 21, the predicted-protein-of-Ostreococcus-lucimarinus-CCE9901 ortholog comprises SEQ ID NO: 22, the predicted-protein-of-Physcomitrella patens-subsp.-patens ortholog comprises SEQ ID NO: 23, the predicted-protein-of-Populus-trichocarpa ortholog comprises SEQ ID NO: 24, the hypothetical-protein-isoform-1-of-Vitis-vinifera ortholog comprises SEQ ID NO: 25, the nascent polypeptide associated complex alpha comprises SEQ ID NO: 26, the proline iminopeptidase comprises SEQ ID NO: 27, the protein transporter comprises SEQ ID NO: 28, the putative-NBS-LRR-disease-resistance-protein-homologue-of-Oryza-sativa-Japonica-Group ortholog comprises SEQ ID NO: 29, the Ran GTPase binding protein comprises SEQ ID NO: 30, the chloroplastic triosephosphate isomerase comprises SEQ ID NO: 31, the V-type proton ATPase catalytic subunit A comprises SEQ ID NO: 32, the regulator of ribonuclease activity A comprises SEQ ID NO: 33, the retroelement pol polyprotein-like ortholog comprises SEQ ID NO: 34, the ribosomal protein L10 comprises SEQ ID NO: 35, the short chain type dehydrogenase comprises SEQ ID NO: 36, the temperature-induced lipocalin comprises SEQ ID NO: 37, and the unknown-protein-of-Picea-sitchensis ortholog comprises SEQ ID NO: 38.

The above-noted proteins can be grouped according to function, e.g. lipid metabolism, non-lipid metabolism, and functions other than metabolism. For example, biotin carboxylase precursor and fructose-bisphosphate aldolase function primarily in lipid metabolism. In contrast, 5-methyltetrahydropteroyltriglutamate-homocysteine methyltransferase, caffeic acid O-methyltransferase, catalase 2, glyceraldehyde 3-phosphate dehydrogenase, large subunit of ribulose-1,5-bisphosphate carboxylase/oxygenase, methionine synthase protein, proline iminopeptidase, Ran GTPase binding protein, chloroplastic triosephosphate isomerase, and V-type proton ATPase catalytic subunit A function primarily in non-lipid metabolism. Also in contrast, the remaining proteins, abscisic stress ripening protein, actin 6, actin E, conserved-hypothetical-protein-of-Ricinus-communis ortholog, fibrillin-like protein, flavodoxin-like quinone reductase 1, H0825G02.11 ortholog, LealP, mitochondrial peroxiredoxin, Os040753300 ortholog, Os05g0482700 ortholog, Os12g0163700 ortholog, OSJNBb0085F13.17 ortholog, predicted-protein-of-Ostreococcus-lucimarinus-CCE9901 ortholog, predicted-protein-of-Physcomitrella patens-subsp.-patens ortholog, predicted-protein-of-Populus-trichocarpa ortholog, hypothetical-protein-isoform-1-of-Vitis-vinifera ortholog, nascent polypeptide associated complex alpha, protein transporter, putative-NBS-LRR-disease-resistance-protein-homologue-of-Oryza-sativa-Japonica-Group ortholog, regulator of ribonuclease activity A, retroelement pol polyprotein-like ortholog, ribosomal protein L10, short chain type dehydrogenase, temperature-induced lipocalin, and unknown-protein-of-Picea-sitchensis ortholog, function primarily in non-metabolic capacities.

Accordingly, in some embodiments the protein is a protein that functions primarily in lipid metabolism selected from the group consisting of biotin carboxylase precursor and fructose-bisphosphate aldolase. Moreover, in some embodiments the protein is a protein that functions primarily in non-lipid metabolism selected from the group consisting of 5-methyltetrahydropteroyltriglutamate-homocysteine methyltransferase, caffeic acid O-methyltransferase, catalase 2, glyceraldehyde 3-phosphate dehydrogenase, large subunit of ribulose-1,5-bisphosphate carboxylase/oxygenase, methionine synthase protein, proline iminopeptidase, Ran GTPase binding protein, chloroplastic triosephosphate isomerase, and V-type proton ATPase catalytic subunit A. In addition, in some embodiments the protein is a protein that functions primarily in a non-metabolic capacity selected from the group consisting of abscisic stress ripening protein, actin 6, actin E, conserved-hypothetical-protein-of-Ricinus-communis ortholog, fibrillin-like protein, flavodoxin-like quinone reductase 1, H0825G02.11 ortholog, LealP, mitochondrial peroxiredoxin, Os02g0753300 ortholog, Os05g0482700 ortholog, Os12g0163700 ortholog, OSJNBb0085F13.17 ortholog, predicted-protein-of-Ostreococcus-lucimarinus-CCE9901 ortholog, predicted-protein-of-Physcomitrella patens-subsp.-patens ortholog, predicted-protein-of-Populus-trichocarpa ortholog, hypothetical-protein-isoform-1-of-Vitis-vinifera ortholog, nascent polypeptide associated complex alpha, protein transporter, putative-NBS-LRR-disease-resistance-protein-homologue-of-Oryza-sativa-Japonica-GrouP ortholog, regulator of ribonuclease activity A, retroelement pol polyprotein-like ortholog, ribosomal protein L10, short chain type dehydrogenase, temperature-induced lipocalin, and unknown-protein-of-Picea-sitchensis ortholog.

### Determining Level of Protein

The level of one of the above-noted proteins in mesocarp tissue of a fruit of a parental oil palm plant may be determined in a preparation of proteins from mesocarp tissue, e.g. a crude preparation, a minimally purified preparation, or a highly purified preparation of mesocarp proteins. The preparation may include total mesocarp proteins, or a subset of mesocarp proteins, e.g. soluble proteins, insoluble proteins, proteins having an isoelectric point between pH 4 to 7, or proteins having higher or lower isoelectric points. The mesocarp tissue itself may be obtained and tested at a particular developmental stage of the fruit from which it is derived, at any time following pollination (“post-pollination”), e.g. 11-19 weeks post-pollination, 11-17 weeks post-pollination, 15-19 weeks post-pollination, 11-13 weeks post-pollination, 15-17 weeks post-pollination, 17-19 weeks post-pollination, 12 weeks post-pollination, 16 weeks post-pollination, or 18 weeks post-pollination. The level of the protein may be expressed in absolute quantitative terms, e.g. mass protein per mass mesocarp tissue, or in relative terms, e.g. intensity of signal of protein relative to intensity of signal of reference.

In some embodiments the step of determining the level of the protein in mesocarp tissue of a fruit of a parental oil palm plant is carried out by antibody-based detection, for example by immunoblot, dot-blot, or enzyme-lined immunosorbent assay, in accordance with methods that are well known in the art. The antibody-based detection may be carried out, for example, by use of monoclonal antibodies or polyclonal antibodies raised against the protein. The antibodies may be prepared by methods that are well known in the art or obtained from commercial vendors. The antibody-based detection may be carried out quantitatively.

In some embodiments the step of determining the level of the protein in mesocarp tissue of a fruit of a parental oil palm plant is carried out by fluorescence-based detection, for example by CyDye labeling of total proteins in a sample, followed by separation and detection of the protein, e.g. by DIGE preparative gel analysis, in accordance with known methods. In some embodiments, levels are determined for more than one of the above-noted proteins. For example, in some embodiments levels are determined for a combination of two to thirty-eight of the above-noted proteins. Also for example, in some embodiments levels are determined for combinations of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38 of the above-noted proteins. By way of example with respect to determining levels for a combination of two of the above-noted proteins, in some embodiments levels are determined for 5-methyltetrahydropteroyltriglutamate-homocysteine methyltransferase and one of the following: abscisic stress ripening protein, actin 6, actin E, biotin carboxylase precursor, caffeic acid O-methyltransferase, catalase 2, conserved-hypothetical-protein-of-Ricinus-communis ortholog, fibrillin-like protein, flavodoxin-like quinone reductase 1, fructose-bisphosphate aldolase, glyceraldehyde 3-phosphate dehydrogenase, H0825G02.11 ortholog, large subunit of ribulose-1,5-bisphosphate carboxylase/oxygenase, LealP, methionine synthase protein, mitochondrial peroxiredoxin, Os02g0753300 ortholog, Os05g0482700 ortholog, Os12g0163700 ortholog, OSJNBb0085F13.17 ortholog, predicted-protein-of-Ostreococcus-lucimarinus-CCE9901 ortholog, predicted-protein-of-Physcomitrella patens-subsp.-patens ortholog, predicted-protein-of-Populus-trichocarpa ortholog, hypothetical-protein-isoform-1-of-Vitis-vinifera ortholog, nascent polypeptide associated complex alpha, proline iminopeptidase, protein transporter, putative-NBS-LRR-disease-resistance-protein-homologue-of-Oryza-sativa-Japonica-Group ortholog, Ran GTPase binding protein, chloroplastic triosephosphate isomerase, V-type proton ATPase catalytic subunit A, regulator of ribonuclease activity A, retroelement pol polyprotein-like ortholog, ribosomal protein L10, short chain type dehydrogenase, temperature-induced lipocalin, or unknown-protein-of-Picea-sitchensis ortholog. In other embodiments levels are determined for each of the other possible combinations of the above-noted proteins.

### Determining Difference Between Levels of Protein

The step of determining whether there is a difference between the level of one of the above-noted proteins in the mesocarp tissue of the fruit of the parental oil palm plant and the level of the protein in mesocarp tissue of a fruit of a reference oil palm plant may be carried out by comparing the respective levels of the protein, for example as determined by antibody-based or fluorescence-based detection as described above, and checking for a difference therebetween. In some embodiments such a comparison is considered to reveal a biologically and/or statistically significant difference based, for example, on the level of the protein in the mesocarp tissue of the parental oil palm being higher (or alternatively, lower) than that of the reference oil palm plant by, for example, greater than 1.1 fold, 1.25 fold, 1.5 fold, 2 fold, 4 fold, or more, with p values of, for example, <0.025, <0.05, or <0.1. As will be apparent to one of ordinary skill, the comparison may be facilitated by use of software for determining and comparing signal intensities, for example by use of Image Quant software (version 6.0, Amersham BioSciences), followed by Biological Variation Analysis using DeCyder™ 2D software version 6.5 (Amersham BioSciences).

In some embodiments, the difference between the level of the protein in the mesocarp tissue of the fruit of the parental oil palm plant and the level of the protein in the mesocarp tissue of the fruit of the reference oil palm plant is that the level of the 5-methyltetrahydropteroyltriglutamate-homocysteine methyltransferase in the mesocarp tissue of the fruit of the parental oil palm plant 11 to 13 weeks after pollination thereof is higher than the level of the 5-methyltetrahydropteroyltriglutamate-homocysteine methyltransferase in the mesocarp tissue of the fruit of the reference oil palm plant 11 to 13 weeks after pollination thereof. For example, in some embodiments the difference is that the level of the 5-methyltetrahydropteroyltriglutamate-homocysteine methyltransferase in the mesocarp tissue of the fruit of the parental oil palm plant 12 weeks after pollination thereof is higher than the level of the 5-methyltetrahydropteroyltriglutamate-homocysteine methyltransferase in the mesocarp tissue of the fruit of the reference oil palm plant 12 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the abscisic stress ripening protein in the mesocarp tissue of the fruit of the parental oil palm plant 11 to 13 weeks after pollination thereof is higher than the level of the abscisic stress ripening protein in the mesocarp tissue of the fruit of the reference oil palm plant 15 to 19 weeks after pollination thereof. For example, in some embodiments the difference is that the level of the abscisic stress ripening protein in the mesocarp tissue of the fruit of the parental oil palm plant 12 weeks after pollination thereof is higher than the level of the abscisic stress ripening protein in the mesocarp tissue of the fruit of the reference oil palm plant 16 or 18 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the actin 6 in the mesocarp tissue of the fruit of the parental oil palm plant 15 to 17 weeks after pollination thereof is lower than the level of the actin 6 in the mesocarp tissue of the fruit of the reference oil palm plant 15 to 17 weeks after pollination thereof. For example, in some embodiments the difference is that the level of the actin 6 in the mesocarp tissue of the fruit of the parental oil palm plant 16 weeks after pollination thereof is lower than the level of the actin 6 in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the actin E in the mesocarp tissue of the fruit of the parental oil palm plant 15 to 19 weeks after pollination thereof is higher than the level of the actin E in the mesocarp tissue of the fruit of the reference oil palm plant 11 to 13 weeks after pollination thereof. For example, in some embodiments the difference is that the level of the actin E in the mesocarp tissue of the fruit of the parental oil palm plant 16 or 18 weeks after pollination thereof is higher than the level of the actin E in the mesocarp tissue of the fruit of the reference oil palm plant 12 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the biotin carboxylase precursor in the mesocarp tissue of the fruit of the parental oil palm plant 15 to 19 weeks after pollination thereof is higher than the level of the biotin carboxylase precursor in the mesocarp tissue of the fruit of the reference oil palm plant 11 to 13 weeks after pollination thereof. For example, in some embodiments the difference is that the level of the biotin carboxylase precursor in the mesocarp tissue of the fruit of the parental oil palm plant 16 or 18 weeks after pollination thereof is higher than the level of the biotin carboxylase precursor in the mesocarp tissue of the fruit of the reference oil palm plant 12 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of caffeic acid O-methyltransferase in the mesocarp tissue of the fruit of the parental oil palm plant 11 to 17 weeks after pollination thereof is lower than the level of caffeic acid O-methyltransferase in the mesocarp tissue of the fruit of the reference oil palm plant 11 to 17 weeks after pollination thereof. For example, in some embodiments the difference is that the level of caffeic acid O-methyltransferase in the mesocarp tissue of the fruit of the parental oil palm plant 12 weeks after pollination thereof is lower than the level of caffeic acid O-methyltransferase in the mesocarp tissue of the fruit of the reference oil palm plant 12 weeks after pollination thereof. Also for example, in some embodiments the difference is that the level of caffeic acid O-methyltransferase in the mesocarp tissue of the fruit of the parental oil palm plant 16 weeks after pollination thereof is lower than the level of caffeic acid O-methyltransferase in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the catalase 2 in the mesocarp tissue of the fruit of the parental oil palm plant 11 to 19 weeks after pollination thereof is higher than the level of the catalase 2 in the mesocarp tissue of the fruit of the reference oil palm plant 11 to 19 weeks after pollination thereof. For example, in some embodiments the difference is that the level of the catalase 2 in the mesocarp tissue of the fruit of the parental oil palm plant 12 weeks after pollination thereof is higher than the level of the catalase 2 in the mesocarp tissue of the fruit of the reference oil palm plant 12 weeks after pollination thereof. Also for example, in some embodiments the difference is that the level of the catalase 2 in the mesocarp tissue of the fruit of the parental oil palm plant 18 weeks after pollination thereof is higher than the level of the catalase 2 in the mesocarp tissue of the fruit of the reference oil palm plant 18 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the conserved-hypothetical-protein-of-Ricinus-communis ortholog in the mesocarp tissue of the fruit of the parental oil palm plant 15 to 17 weeks after pollination thereof is higher than the level of the conserved-hypothetical-protein-of-Ricinus-communis ortholog in the mesocarp tissue of the fruit of the reference oil palm plant 15 to 17 weeks after pollination thereof. For example, in some embodiments the difference is that the level of the conserved-hypothetical-protein-of-Ricinus-communis ortholog in the mesocarp tissue of the fruit of the parental oil palm plant 16 weeks after pollination thereof is higher than the level of the conserved-hypothetical-protein-of-Ricinus-communis ortholog in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the fibrillin-like protein in the mesocarp tissue of the fruit of the parental oil palm plant 11 to 13 weeks after pollination thereof is higher than the level of the fibrillin-like protein in the mesocarp tissue of the fruit of the reference oil palm plant 11 to 13 weeks after pollination thereof. For example, in some embodiments the difference is that the level of the fibrillin-like protein in the mesocarp tissue of the fruit of the parental oil palm plant 12 weeks after pollination thereof is higher than the level of the fibrillin-like protein in the mesocarp tissue of the fruit of the reference oil palm plant 12 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the difference is that the level of the flavodoxin-like quinone reductase 1 in the mesocarp tissue of the fruit of the parental oil palm plant 11 to 19 weeks after pollination thereof is lower than the level of the flavodoxin-like quinone reductase 1 in the mesocarp tissue of the fruit of the reference oil palm plant 11 to 19 weeks after pollination thereof. For example, in some embodiments the difference is that the level of the flavodoxin-like quinone reductase 1 in the mesocarp tissue of the fruit of the parental oil palm plant 12 weeks after pollination thereof is lower than the level of the flavodoxin-like quinone reductase 1 in the mesocarp tissue of the fruit of the reference oil palm plant 12 weeks after pollination thereof. Also for example, in some embodiments the difference is that the level of the flavodoxin-like quinone reductase 1 in the mesocarp tissue of the fruit of the parental oil palm plant 18 weeks after pollination thereof is lower than the level of the flavodoxin-like quinone reductase 1 in the mesocarp tissue of the fruit of the reference oil palm plant 18 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the fructose-bisphosphate aldolase in the mesocarp tissue of the fruit of the parental oil palm plant 15 to 17 weeks after pollination thereof is higher than the level of the fructose-bisphosphate aldolase in the mesocarp tissue of the fruit of the reference oil palm plant 15 to 17 weeks after pollination thereof. For example, in some embodiments the difference is that the level of the fructose-bisphosphate aldolase in the mesocarp tissue of the fruit of the parental oil palm plant 16 weeks after pollination thereof is higher than the level of the fructose-bisphosphate aldolase in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the glyceraldehyde 3-phosphate dehydrogenase in the mesocarp tissue of the fruit of the parental oil palm plant 15 to 17 weeks after pollination thereof is lower than the level of the glyceraldehyde 3-phosphate dehydrogenase in the mesocarp tissue of the fruit of the reference oil palm plant 15 to 17 weeks after pollination thereof. For example, in some embodiments the difference is that the level of the glyceraldehyde 3-phosphate dehydrogenase in the mesocarp tissue of the fruit of the parental oil palm plant 16 weeks after pollination thereof is lower than the level of the glyceraldehyde 3-phosphate dehydrogenase in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the H0825002.11 ortholog in the mesocarp tissue of the fruit of the parental oil palm plant 15 to 17 weeks after pollination thereof is lower than the level of the H0825002.11 ortholog in the mesocarp tissue of the fruit of the reference oil palm plant 15 to 17 weeks after pollination thereof. For example, in some embodiments the difference is that the level of the H0825002.11 ortholog in the mesocarp tissue of the fruit of the parental oil palm plant 16 weeks after pollination thereof is lower than the level of the H0825G02.11 ortholog in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the large subunit of ribulose-1,5-bisphosphate carboxylase/oxygenase in the mesocarp tissue of the fruit of the parental oil palm plant 17 to 19 weeks after pollination thereof is higher than the level of the large subunit of ribulose-1,5-bisphosphate carboxylase/oxygenase in the mesocarp tissue of the fruit of the reference oil palm plant 17 to 19 weeks after pollination thereof. For example, in some embodiments the difference is that the level of the large subunit of ribulose-1,5-bisphosphate carboxylase/oxygenase in the mesocarp tissue of the fruit of the parental oil palm plant 18 weeks after pollination thereof is higher than the level of the large subunit of ribulose-1,5-bisphosphate carboxylase/oxygenase in the mesocarp tissue of the fruit of the reference oil palm plant 18 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the LealP in the mesocarp tissue of the fruit of the parental oil palm plant 15 to 17 weeks after pollination thereof is higher than the level of the LealP in the mesocarp tissue of the fruit of the reference oil palm plant 15 to 17 weeks after pollination thereof. For example, in some embodiments the difference is that the level of the LealP in the mesocarp tissue of the fruit of the parental oil palm plant 16 weeks after pollination thereof is higher than the level of the LealP in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the methionine synthase protein in the mesocarp tissue of the fruit of the parental oil palm plant 15 to 17 weeks after pollination thereof is lower than the level of the methionine synthase protein in the mesocarp tissue of the fruit of the reference oil palm plant 15 to 17 weeks after pollination thereof. For example, in some embodiments the difference is that the level of the methionine synthase protein in the mesocarp tissue of the fruit of the parental oil palm plant 16 weeks after pollination thereof is lower than the level of the methionine synthase protein in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the mitochondrial peroxiredoxin in the mesocarp tissue of the fruit of the parental oil palm plant 11 to 17 weeks after pollination thereof is higher than the level of the mitochondrial peroxiredoxin in the mesocarp tissue of the fruit of the reference oil palm plant 11 to 17 weeks after pollination thereof. For example, in some embodiments the difference is that the level of the mitochondrial peroxiredoxin in the mesocarp tissue of the fruit of the parental oil palm plant 12 weeks after pollination thereof is higher than the level of the mitochondrial peroxiredoxin in the mesocarp tissue of the fruit of the reference oil palm plant 12 weeks after pollination thereof. Also for example, in some embodiments the difference is that the level of the mitochondrial peroxiredoxin in the mesocarp tissue of the fruit of the parental oil palm plant 16 weeks after pollination thereof is higher than the level of the mitochondrial peroxiredoxin in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the Os02g0753300 ortholog in the mesocarp tissue of the fruit of the parental oil palm plant 15 to 17 weeks after pollination thereof is higher than the level of the Os02g0753300 ortholog in the mesocarp tissue of the fruit of the reference oil palm plant 15 to 17 weeks after pollination thereof. For example, in some embodiments the difference is that the level of the Os02g0753300 ortholog in the mesocarp tissue of the fruit of the parental oil palm plant 16 weeks after pollination thereof is higher than the level of the Os02g0753300 ortholog in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the Os05g0482700 ortholog in the mesocarp tissue of the fruit of the parental oil palm plant 15 to 17 weeks after pollination thereof is lower than the level of the Os05g0482700 ortholog in the mesocarp tissue of the fruit of the reference oil palm plant 15 to 17 weeks after pollination thereof. For example, in some embodiments the difference is that the level of the Os05g0482700 ortholog in the mesocarp tissue of the fruit of the parental oil palm plant 16 weeks after pollination thereof is lower than the level of the Os05g0482700 ortholog in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the Os12g0163700 ortholog in the mesocarp tissue of the fruit of the parental oil palm plant 15 to 19 weeks after pollination thereof is higher than the level of the Os12g0163700 ortholog in the mesocarp tissue of the fruit of the reference oil palm plant 11 to 13 weeks after pollination thereof. For example, in some embodiments the difference is that the level of the Os12g0163700 ortholog in the mesocarp tissue of the fruit of the parental oil palm plant 16 or 18 weeks after pollination thereof is higher than the level of the Os12g0163700 ortholog in the mesocarp tissue of the fruit of the reference oil palm plant 12 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the OSJNBb0085F13.17 ortholog in the mesocarp tissue of the fruit of the parental oil palm plant 15 to 17 weeks after pollination thereof is lower than the level of the OSJNBb0085F13.17 ortholog in the mesocarp tissue of the fruit of the reference oil palm plant 15 to 17 weeks after pollination thereof. For example, in some embodiments the difference is that the level of the OSJNBb0085F13.17 ortholog in the mesocarp tissue of the fruit of the parental oil palm plant 16 weeks after pollination thereof is lower than the level of the OSJNBb0085F13.17 ortholog in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the predicted-protein-of-Ostreococcus-lucimarinus-CCE9901 ortholog in the mesocarp tissue of the fruit of the parental oil palm plant 15 to 17 weeks after pollination thereof is higher than the level of the predicted-protein-of-Ostreococcus-lucimarinus-CCE9901 ortholog in the mesocarp tissue of the fruit of the reference oil palm plant 15 to 17 weeks after pollination thereof. For example, in some embodiments the difference is that the level of the predicted-protein-of-Ostreococcus-lucimarinus-CCE9901 ortholog in the mesocarp tissue of the fruit of the parental oil palm plant 16 weeks after pollination thereof is higher than the level of the predicted-protein-of-Ostreococcus-lucimarinus-CCE9901 ortholog in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the predicted-protein-of-Physcomitrella patens-subsp.-patens ortholog in the mesocarp tissue of the fruit of the parental oil palm plant 11 to 13 weeks after pollination thereof is lower than the level of the predicted-protein-of-Physcomitrella patens-subsp.-patens ortholog in the mesocarp tissue of the fruit of the reference oil palm plant 11 to 13 weeks after pollination thereof. For example, in some embodiments the difference is that the level of the predicted-protein-of-Physcomitrella patens-subsp.-patens ortholog in the mesocarp tissue of the fruit of the parental oil palm plant 12 weeks after pollination thereof is lower than the level of the predicted-protein-of-Physcomitrella patens-subsp.-patens ortholog in the mesocarp tissue of the fruit of the reference oil palm plant 12 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the predicted-protein-of-Populus-trichocarpa ortholog in the mesocarp tissue of the fruit of the parental oil palm plant 11 to 17 weeks after pollination thereof is lower than the level of the predicted-protein-of-Populus-trichocarpa ortholog in the mesocarp tissue of the fruit of the reference oil palm plant 11 to 17 weeks after pollination thereof. For example, in some embodiments the difference is that the level of the predicted-protein-of-Populus-trichocarpa ortholog in the mesocarp tissue of the fruit of the parental oil palm plant 12 weeks after pollination thereof is lower than the level of the predicted-protein-of-Populus-trichocarpa ortholog in the mesocarp tissue of the fruit of the reference oil palm plant 12 weeks after pollination thereof. Also for example, in some embodiments the difference is that the level of the predicted-protein-of-Populus-trichocarpa ortholog in the mesocarp tissue of the fruit of the parental oil palm plant 16 weeks after pollination thereof is lower than the level of the predicted-protein-of-Populus-trichocarpa ortholog in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the hypothetical-protein-isoform-1-of-Vitis-vinifera ortholog in the mesocarp tissue of the fruit of the parental oil palm plant 11 to 13 weeks after pollination thereof is higher than the level of the hypothetical-protein-isoform-1-of-Vitis-vinifera ortholog in the mesocarp tissue of the fruit of the reference oil palm plant 11 to 13 weeks after pollination thereof. For example, in some embodiments the difference is that the level of the hypothetical-protein-isoform-1-of-Vitis-vinifera ortholog in the mesocarp tissue of the fruit of the parental oil palm plant 12 weeks after pollination thereof is higher than the level of the hypothetical-protein-isoform-1-of-Vitis-vinifera ortholog in the mesocarp tissue of the fruit of the reference oil palm plant 12 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the nascent polypeptide associated complex alpha in the mesocarp tissue of the fruit of the parental oil palm plant 11 to 19 weeks after pollination thereof is higher than the level of the nascent polypeptide associated complex alpha in the mesocarp tissue of the fruit of the reference oil palm plant 11 to 19 weeks after pollination thereof. For example, in some embodiments the difference is that the level of the nascent polypeptide associated complex alpha in the mesocarp tissue of the fruit of the parental oil palm plant 12 weeks after pollination thereof is higher than the level of the nascent polypeptide associated complex alpha in the mesocarp tissue of the fruit of the reference oil palm plant 12 weeks after pollination thereof. Also for example, in some embodiments the difference is that the level of the nascent polypeptide associated complex alpha in the mesocarp tissue of the fruit of the parental oil palm plant 18 weeks after pollination thereof is higher than the level of the nascent polypeptide associated complex alpha in the mesocarp tissue of the fruit of the reference oil palm plant 18 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the proline iminopeptidase in the mesocarp tissue of the fruit of the parental oil palm plant 15 to 19 weeks after pollination thereof is higher than the level of the proline iminopeptidase in the mesocarp tissue of the fruit of the reference oil palm plant 15 to 19 weeks after pollination thereof. For example, in some embodiments the difference is that the level of the proline iminopeptidase in the mesocarp tissue of the fruit of the parental oil palm plant 16 weeks after pollination thereof is higher than the level of the proline iminopeptidase in the mesocarp tissue of the fruit of the reference oil palm plant 16 weeks after pollination thereof. Also for example, in some embodiments the difference is that the level of the proline iminopeptidase in the mesocarp tissue of the fruit of the parental oil palm plant 18 weeks after pollination thereof is higher than the level of the proline iminopeptidase in the mesocarp tissue of the fruit of the reference oil palm plant 18 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the protein transporter in the mesocarp tissue of the fruit of the parental oil palm plant 11 to 13 weeks after pollination thereof is lower than the level of the protein transporter in the mesocarp tissue of the fruit of the reference oil palm plant 11 to 13 weeks after pollination thereof. For example, in some embodiments the difference is that the level of the protein transporter in the mesocarp tissue of the fruit of the parental oil palm plant 12 weeks after pollination thereof is lower than the level of the protein transporter in the mesocarp tissue of the fruit of the reference oil palm plant 12 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the putative-NBS-LRR-disease-resistance-protein-homologue-of-Oryza-sativa-Japonica-Group ortholog in the mesocarp tissue of the fruit of the parental oil palm plant 15 to 17 weeks after pollination thereof is lower than the level of the putative-NBS-LRR-disease-resistance-protein-homologue-of-Oryza-sativa-Japonica-Group ortholog in the mesocarp tissue of the fruit of the reference oil palm plant 15 to 17 weeks after pollination thereof. For example, in some embodiments the difference is that the level of the putative-NBS-LRR-disease-resistance-protein-homologue-of-Oryza-sativa-Japonica-Group ortholog in the mesocarp tissue of the fruit of the parental oil palm plant 16 weeks after pollination thereof is lower than the level of the putative-NBS-LRR-disease-resistance-protein-homologue-of-Oryza-sativa-Japonica-Group ortholog in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the Ran GTPase binding protein in the mesocarp tissue of the fruit of the parental oil palm plant 11 to 13 weeks after pollination thereof is higher than the level of the Ran GTPase binding protein in the mesocarp tissue of the fruit of the reference oil palm plant 11 to 13 weeks after pollination thereof. For example, in some embodiments the difference is that the level of the Ran GTPase binding protein in the mesocarp tissue of the fruit of the parental oil palm plant 12 weeks after pollination thereof is higher than the level of the Ran GTPase binding protein in the mesocarp tissue of the fruit of the reference oil palm plant 12 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the chloroplastic triosephosphate isomerase in the mesocarp tissue of the fruit of the parental oil palm plant 15 to 17 weeks after pollination thereof is lower than the level of the chloroplastic triosephosphate isomerase in the mesocarp tissue of the fruit of the reference oil palm plant 15 to 17 weeks after pollination thereof. For example, in some embodiments the difference is that the level of the chloroplastic triosephosphate isomerase in the mesocarp tissue of the fruit of the parental oil palm plant 16 weeks after pollination thereof is lower than the level of the chloroplastic triosephosphate isomerase in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the V-type proton ATPase catalytic subunit A in the mesocarp tissue of the fruit of the parental oil palm plant 15 to 17 weeks after pollination thereof is lower than the level of the V-type proton ATPase catalytic subunit A in the mesocarp tissue of the fruit of the reference oil palm plant 15 to 17 weeks after pollination thereof. For example, in some embodiments the difference is that the level of the V-type proton ATPase catalytic subunit A in the mesocarp tissue of the fruit of the parental oil palm plant 16 weeks after pollination thereof is lower than the level of the V-type proton ATPase catalytic subunit A in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the regulator of ribonuclease activity A in the mesocarp tissue of the fruit of the parental oil palm plant 15 to 19 weeks after pollination thereof is higher than the level of the regulator of ribonuclease activity A in the mesocarp tissue of the fruit of the reference oil palm plant 11 to 13 weeks after pollination thereof. For example, in some embodiments the difference is that the level of the regulator of ribonuclease activity A in the mesocarp tissue of the fruit of the parental oil palm plant 16 or 18 weeks after pollination thereof is higher than the level of the regulator of ribonuclease activity A in the mesocarp tissue of the fruit of the reference oil palm plant 12 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the retroelement pol polyprotein-like ortholog in the mesocarp tissue of the fruit of the parental oil palm plant 11 to 13 weeks after pollination thereof is lower than the level of the retroelement pol polyprotein-like ortholog in the mesocarp tissue of the fruit of the reference oil palm plant 11 to 13 weeks after pollination thereof. For example, in some embodiments the difference is that the level of the retroelement pol polyprotein-like ortholog in the mesocarp tissue of the fruit of the parental oil palm plant 12 weeks after pollination thereof is lower than the level of the retroelement pol polyprotein-like ortholog in the mesocarp tissue of the fruit of the reference oil palm plant 12 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the ribosomal protein L 10 in the mesocarp tissue of the fruit of the parental oil palm plant 11 to 13 weeks after pollination thereof is lower than the level of the ribosomal protein L10 in the mesocarp tissue of the fruit of the reference oil palm plant 11 to 13 weeks after pollination thereof. For example, in some embodiments the difference is that the level of the ribosomal protein L10 in the mesocarp tissue of the fruit of the parental oil palm plant 12 weeks after pollination thereof is lower than the level of the ribosomal protein L10 in the mesocarp tissue of the fruit of the reference oil palm plant 12 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the short chain type dehydrogenase in the mesocarp tissue of the fruit of the parental oil palm plant 15 to 19 weeks after pollination thereof is higher than the level of the short chain type dehydrogenase in the mesocarp tissue of the fruit of the reference oil palm plant 11 to 13 weeks after pollination thereof. For example, in some embodiments the difference is that the level of the short chain type dehydrogenase in the mesocarp tissue of the fruit of the parental oil palm plant 16 or 18 weeks after pollination thereof is higher than the level of the short chain type dehydrogenase in the mesocarp tissue of the fruit of the reference oil palm plant 12 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the temperature-induced lipocalin in the mesocarp tissue of the fruit of the parental oil palm plant 15 to 17 weeks after pollination thereof is higher than the level of the temperature-induced lipocalin in the mesocarp tissue of the fruit of the reference oil palm plant 15 to 17 weeks after pollination thereof. For example, in some embodiments the difference is that the level of the temperature-induced lipocalin in the mesocarp tissue of the fruit of the parental oil palm plant 16 weeks after pollination thereof is higher than the level of the temperature-induced lipocalin in the mesocarp tissue of the fruit of the reference oil palm plant 16 weeks after pollination thereof.

In some embodiments, the difference between the level of the protein in the mesocarp tissue of the fruit of the parental oil palm plant and the level of the protein in the mesocarp tissue of the fruit of the reference oil palm plant is that the level of the unknown-protein-of-Picea-sitchensis ortholog in the mesocarp tissue of the fruit of the parental oil palm plant 15 to 17 weeks after pollination thereof is higher than the level of the unknown-protein-of-Picea-sitchensis ortholog in the mesocarp tissue of the fruit of the reference oil palm plant 15 to 17 weeks after pollination thereof. For example, in some embodiments the difference is that the level of the unknown-protein-of-Picea-sitchensis ortholog in the mesocarp tissue of the fruit of the parental oil palm plant 16 weeks after pollination thereof is higher than the level of the unknown-protein-of-Picea-sitchensis ortholog in the mesocarp tissue of the fruit of the reference oil palm plant 16 weeks after pollination thereof.

In some embodiments, differences between the level of the protein in the mesocarp tissue of the fruit of the parental oil palm plant and the level of the protein in the mesocarp tissue of the fruit of the reference oil palm plant are determined for more than one of the above-noted proteins. For example, in some embodiments differences are determined for a combination of two to thirty-eight of the above-noted proteins. Also for example, in some embodiments differences are determined for combinations of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38 of the above-noted proteins, e.g. each possible combination.

### Selecting Progeny

The step of selecting progeny of the parental oil palm plant based on the difference between the level of the protein in the mesocarp tissue of the fruit of the parental oil palm plant and the level of the protein in mesocarp tissue of the fruit of the reference oil palm plant to obtain the high-yielding oil palm plant may be carried out, for example, by choosing a parental oil palm plant for propagation based on the difference and crossing the plant with another oil palm plant, e.g. another oil palm plant also exhibiting the same or a similar difference with respect to one of the above-noted proteins, by conventional breeding techniques to obtain progeny corresponding to the high-yielding oil palm plant.

As is well known in the art, fruit type is a monogenic trait in oil palm that is important with respect to breeding and commercial production of palm oil. Specifically, oil palms with either of two distinct fruit types are generally used in breeding and seed production through crossing in order to generate palms for commercial production of oil (also termed “commercial planting materials” or “agricultural production plants”). The first fruit type is dura (genotype: sh+ sh+), which is characterized by a thick shell corresponding to 28 to 35% of the fruit by weight, with no ring of black fibres around the kernel of the fruit. For dura fruits, the mesocarp to fruit ratio varies from 50 to 60%, with extractable oil content in proportion to bunch weight of 18 to 24%. The second fruit type is pisifera (genotype: sh− sh−), which is characterized by the absence of a shell, the vestiges of which are represented by a ring of fibres around a small kernel. Accordingly, for pisifera fruits, the mesocarp to fruit ratio is 90 to 100%. The mesocarp oil to bunch ratio is comparable to the dura at 16 to 28%. Pisiferas are however usually female sterile as the majority of bunches abort at an early stage of development.

Crossing dura and pisifera gives rise to palms with a third fruit type, the tenera (genotype: sh+ sh−). Tenera fruits have thin shells of 8 to 10% of the fruit by weight, corresponding to a thickness of 0.5 to 4 mm, around which is a characteristic ring of black fibres. For tenera fruits, the ratio of mesocarp to fruit is comparatively high, in the range of 60 to 80%. Commercial tenera palms generally produce more fruit bunches than duras, although mean bunch weight is lower. The extractable oil to bunch ratio is in the range of 20 to 30%, the highest of the three fruit types, and thus tenera are typically used as commercial planting materials.

Dura palm breeding populations used in Southeast Asia include Serdang Avenue, Ulu Remis (which incorporated some Serdang Avenue material), Johor Labis, and Elmina estate, including Deli Dumpy, all of which are derived from Deli dura. Pisifera breeding populations used for seed production are generally grouped as Yangambi, AVROS, Bing a and URT. Other dura and pisifera populations are used in Africa and South America.

Accordingly, in some embodiments the parental oil palm plant is a dura palm selected from the group consisting of Deli dura, Serdang Avenue dura, Ulu Remis dura, Johor Labis dura, Elmina estate dura, and Deli Dumpy dura. Alternatively, in some embodiments the parental oil palm plant is a pisifera palm selected from the group consisting of Yangambi pisifera, AVROS pisifera, Bing a pisifera, and URT pisifera.

Oil palm breeding is primarily aimed at selecting for improved parental dura and pisifera breeding stock palms for production of superior tenera commercial planting materials. Such materials are largely in the form of seeds although the use of tissue culture for propagation of clones continues to be developed. Generally, parental dura breeding populations are generated by crossing among selected dura palms. Based on the monogenic inheritance of fruit type, 100% of the resulting palms will be duras. After several years of yield recording and confirmation of bunch and fruit characteristics, duras are selected for breeding based on phenotype. In contrast, pisifera palms are normally female sterile and thus breeding populations thereof must be generated by crossing among selected teneras or by crossing selected teneras with selected pisiferas. The tenera×tenera cross will generate 25% duras, 50% teneras and 25% pisiferas.

The tenera×pisifera cross will generate 50% teneras and 50% pisiferas. The yield potential of pisiferas is then determined indirectly by progeny testing with the elite duras, i.e. by crossing duras and pisiferas to generate teneras, and then determining yield phenotypes of the fruits of the teneras over time. From this, pisiferas with good general combining ability are selected based on the performance of their tenera progenies. Intercrossing among selected parents is also carried out with progenies being carried forward to the next breeding cycle. This allows introduction of new genes into the breeding programme to increase genetic variability. Using this general scheme, priority selection objectives include high oil yield per unit area in terms of high fresh fruit bunch yield and high oil to bunch ratio (thin shell, thick mesocarp), high early yield (precocity), and good oil qualities, among other traits.

Accordingly, in some embodiments, the parental oil palm plant is a dura breeding stock plant, the progeny comprises an oil palm plant selected from the group consisting of a dura breeding stock plant and a tenera agricultural production plant, and the high-yielding oil palm plant is selected from the group consisting of a dura breeding stock plant and a tenera agricultural production plant. For example, in some embodiments the method is carried out with the purpose of generating improved dura breeding stock, in which case the parental dura breeding stock plant is crossed with another dura breeding stock plant to obtain a high yielding oil palm plant directly among the progeny, which will also be dura breeding stock plants. Also for example, in some embodiments the method is carried out with the purpose of generating improved tenera agricultural production plants, in which case the parental dura breeding stock plant is crossed with a pisifera breeding stock plant to obtain a high yielding oil palm plant directly among the progeny, which will be tenera agricultural production plants.

Alternatively, in some embodiments the parental oil palm plant is a tenera breeding stock plant, the progeny comprises an oil palm plant selected from the group consisting of a tenera breeding stock plant, a pisifera breeding stock plant, and a tenera agricultural production plant, and the high-yielding oil palm plant is selected from the group consisting of a tenera breeding stock plant and a tenera agricultural production plant. For example, in some embodiments the method may be carried out with the purpose of generating improved tenera breeding stock, in which case the parental tenera breeding stock plant is crossed with another tenera breeding stock plant, to obtain a tenera high yielding palm plant directly among the progeny, of which 25% will be dura, 50% will be tenera, and 25% will be pisifera. Also for example, in some embodiments the method is carried out with the purpose of generating improved tenera agricultural production plants, in which case the parental tenera breeding stock plant is crossed with a pisifera breeding stock plant, to yield progeny corresponding to 50% tenera and 50% pisifera. The pisifera resulting from this cross can in turn be used as pisifera breeding stock for generation of tenera agricultural production plants.

Progeny plants may be cultivated by conventional approaches, e.g. seedlings may be cultivated in polyethylene bags in pre-nursery and nursery settings, raised for about 12 months, and then planted as seedlings, with progeny that are known or predicted to exhibit high yields chosen for further cultivation.

As will be apparent from the foregoing, the step of selecting progeny of the parental oil palm plant may also be based on differences between levels of more than one of the proteins in the mesocarp tissue of the fruit of the parental oil palm plant and the levels of the proteins in mesocarp tissue of a fruit of a reference oil palm plant to obtain the high-yielding oil palm plant. For example, in some embodiments the step of selecting is based on differences with respect to a combination of two to thirty-eight of the above-noted proteins. Also for example, in some embodiments the step of selecting is based on differences with respect to combinations of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38 of the above-noted proteins, e.g. each possible combination.

### Additional Proteins

In some embodiments, in addition to determining a difference with respect to one or more than one of the above-noted proteins, a difference is also determined with respect to one or more additional proteins selected from the group consisting of 17.6 kDa class I small heat shock protein, ABC1 family protein, glutathione peroxidase, glutathione S-transferase, glutathione-S-transferase theta, phospholipase D, and VIER F-Box Proteine 2. In some embodiments, the 17.6 kDa class I small heat shock protein comprises SEQ ID NO: 39, the ABC1 family protein comprises SEQ ID NO: 40, the glutathione peroxidase comprises SEQ ID NO: 41, the glutathione S-transferase comprises SEQ ID NO: 42, the glutathione-S-transferase theta comprises SEQ ID NO: 43, the phospholipase D comprises SEQ ID NO: 44, and the VIER F-Box Proteine 2 comprises SEQ ID NO: 45.

In some embodiments, the difference between the level of the additional protein in the mesocarp tissue of the fruit of the parental oil palm plant and the level of the additional protein in mesocarp tissue of the fruit of the reference oil palm plant is that the level of the 17.6 kDa class I small heat shock protein in the mesocarp tissue of the fruit of the parental oil palm plant 15 to 19 weeks after pollination thereof is higher than the level of the 17.6 kDa class I small heat shock protein in the mesocarp tissue of the fruit of the reference oil palm plant 11 to 13 weeks after pollination thereof. For example, in some embodiments the difference is that the level of the 17.6 kDa class I small heat shock protein in the mesocarp tissue of the fruit of the parental oil palm plant 16 or 18 weeks after pollination thereof is higher than the level of the 17.6 kDa class I small heat shock protein in the mesocarp tissue of the fruit of the reference oil palm plant 12 weeks after pollination thereof.

In some embodiments, the difference between the level of the additional protein in the mesocarp tissue of the fruit of the parental oil palm plant and the level of the additional protein in the mesocarp tissue of the fruit of the reference oil palm plant is that the level of the ABC1 family protein in the mesocarp tissue of the fruit of the parental oil palm plant 15 to 17 weeks after pollination thereof is higher than the level of the ABC1 family protein in the mesocarp tissue of the fruit of the reference oil palm plant 15 to 17 weeks after pollination thereof. For example, in some embodiments the difference is that the level of the ABC1 family protein in the mesocarp tissue of the fruit of the parental oil palm plant 16 weeks after pollination thereof is higher than the level of the ABC1 family protein in the mesocarp tissue of the fruit of the reference oil palm plant 16 weeks after pollination thereof.

In some embodiments, the difference between the level of the additional protein in the mesocarp tissue of the fruit of the parental oil palm plant and the level of the additional protein in the mesocarp tissue of the fruit of the reference oil palm plant is that the level of the glutathione peroxidase in the mesocarp tissue of the fruit of the parental oil palm plant 15 to 17 weeks after pollination thereof is higher than the level of the glutathione peroxidase in the mesocarp tissue of the fruit of the reference oil palm plant 15 to 17 weeks after pollination thereof. For example, in some embodiments the difference is that the level of the glutathione peroxidase in the mesocarp tissue of the fruit of the parental oil palm plant 16 weeks after pollination thereof is higher than the level of the glutathione peroxidase in the mesocarp tissue of the fruit of the reference oil palm plant 16 weeks after pollination thereof.

In some embodiments, the difference between the level of the additional protein in the mesocarp tissue of the fruit of the parental oil palm plant and the level of the additional protein in the mesocarp tissue of the fruit of the reference oil palm plant is that the level of the glutathione S-transferase in the mesocarp tissue of the fruit of the parental oil palm plant 11 to 19 weeks after pollination thereof is lower than the level of the glutathione S-transferase in the mesocarp tissue of the fruit of the reference oil palm plant 11 to 19 weeks after pollination thereof. For example, in some embodiments the difference is that the level of the glutathione S-transferase in the mesocarp tissue of the fruit of the parental oil palm plant 12 weeks after pollination thereof is lower than the level of the glutathione S-transferase in the mesocarp tissue of the fruit of the reference oil palm plant 12 weeks after pollination thereof. Also for example, in some embodiments the difference is that the level of the glutathione S-transferase in the mesocarp tissue of the fruit of the parental oil palm plant 16 weeks after pollination thereof is lower than the level of the glutathione S-transferase in the mesocarp tissue of the fruit of the reference oil palm plant 16 weeks after pollination thereof. As a further example, in some embodiments the difference is that the level of the glutathione S-transferase in the mesocarp tissue of the fruit of the parental oil palm plant 18 weeks after pollination thereof is lower than the level of the glutathione S-transferase in the mesocarp tissue of the fruit of the reference oil palm plant 18 weeks after pollination thereof.

In some embodiments, the difference between the level of the additional protein in the mesocarp tissue of the fruit of the parental oil palm plant and the level of the additional protein in the mesocarp tissue of the fruit of the reference oil palm plant is that the level of the glutathione-S-transferase theta in the mesocarp tissue of the fruit of the parental oil palm plant 17 to 19 weeks after pollination thereof is higher than the level of the glutathione-S-transferase theta in the mesocarp tissue of the fruit of the reference oil palm plant 17 to 19 weeks after pollination thereof. For example, in some embodiments the difference is that the level of the glutathione-S-transferase theta in the mesocarp tissue of the fruit of the parental oil palm plant 18 weeks after pollination thereof is higher than the level of the glutathione-S-transferase theta in the mesocarp tissue of the fruit of the reference oil palm plant 18 weeks after pollination thereof.

In some embodiments, the difference between the level of the additional protein in the mesocarp tissue of the fruit of the parental oil palm plant and the level of the additional protein in the mesocarp tissue of the fruit of the reference oil palm plant is that the level of the phospholipase D in the mesocarp tissue of the fruit of the parental oil palm plant 15 to 17 weeks after pollination thereof is lower than the level of the phospholipase D in the mesocarp tissue of the fruit of the reference oil palm plant 15 to 17 weeks after pollination thereof. For example, in some embodiments the difference is that the level of the phospholipase D in the mesocarp tissue of the fruit of the parental oil palm plant 16 weeks after pollination thereof is lower than the level of the phospholipase D in the mesocarp tissue of the fruit of the reference oil palm plant 16 weeks after pollination thereof.

In some embodiments, the difference between the level of the additional protein in the mesocarp tissue of the fruit of the parental oil palm plant and the level of the additional protein in the mesocarp tissue of the fruit of the reference oil palm plant is that the level of the VIER F-Box Proteine 2 in the mesocarp tissue of the fruit of the parental oil palm plant 17 to 19 weeks after pollination thereof is higher than the level of the VIER F-Box Proteine 2 in the mesocarp tissue of the fruit of the reference oil palm plant 17 to 19 weeks after pollination thereof. For example, in some embodiments the difference is that the level of the VIER F-Box Protein 2 in the mesocarp tissue of the fruit of the parental oil palm plant 18 weeks after pollination thereof is higher than the level of the VIER F-Box Proteine 2 in the mesocarp tissue of the fruit of the reference oil palm plant 18 weeks after pollination thereof.

### Method for Obtaining Palm Oil

A method for obtaining palm oil from a high-yielding oil palm plant is also disclosed. The method includes the steps of obtaining a high-yielding oil palm plant as explained above; and isolating palm oil from a fruit of the high-yielding oil palm plant. The step of isolating palm oil may be carried out by conventional approaches, e.g. harvesting of fruit bunches followed by extraction of oil, within 24 hours, from the fresh and non-wounded fruits thereof.

### Method for Predicting Oil Yield of a Test Oil Palm Plant

As noted above, also provided is a method for predicting oil yield of a test oil palm plant. The method comprises: (i) determining the level of a protein in mesocarp tissue of a fruit of the test oil palm plant; (ii) determining whether there is a difference between the level of the protein in the mesocarp tissue of the fruit of the test oil palm plant and the level of the protein in mesocarp tissue of a fruit of a reference oil palm plant; and (iii) predicting the oil yield of the test oil palm plant based on the difference.

The proteins described above as being useful in the method for obtaining a high-yielding oil palm plant are also useful in the method for predicting oil yield of a test oil palm plant.

The step of determining the level of a protein in mesocarp tissue of a fruit of the test oil palm plant may also be carried out similarly as described above, e.g. based on two-dimensional fluorescence difference gel electrophoresis, antibody-based detection, immunoblot detection, or dot-blot detection, and/or with respect to more than one of the proteins, except that the level of the protein in the mesocarp tissue of the fruit is determined with respect to a fruit of a test oil palm plant rather than a parental oil palm plant.

The step of determining whether there is a difference between the level of the protein in the mesocarp tissue of the fruit of the test oil palm plant and the level of the protein in the mesocarp tissue of a fruit of a reference oil palm plant may also be carried as described above, based for example on the level of the protein of the mesocarp of the test oil palm plant being higher (or alternatively, lower) than that of the reference oil palm plant by, for example, greater than 1.1 fold, 1.25 fold, 1.5 fold, 2 fold, 4 fold, or more, with p values of, for example, <0.025, <0.05, or <0.1. Moreover, the difference may be based on any of the specific differences noted above with respect to each specific protein, e.g. in some embodiments the difference between the level of the protein in the mesocarp tissue of the fruit of the test oil palm plant and the level of the protein in mesocarp tissue of the fruit of the reference oil palm plant is that the level of the 5-methyltetrahydropteroyltriglutamate-homocysteine methyltransferase in the mesocarp tissue of the fruit of the test oil palm plant 11 to 13 weeks after pollination thereof is higher than the level of the 5-methyltetrahydropteroyltriglutamate-homocysteine methyltransferase in the mesocarp tissue of the fruit of the reference oil palm plant 11 to 13 weeks after pollination thereof. In addition, differences may be determined with respect to levels of more than one of the proteins.

The predicting step may be carried out, for example, based on the amount of the difference in the level of the protein in the mesocarp tissue of the fruit of the test oil palm plant and the level of the protein in mesocarp tissue of the fruit of the reference oil palm plant, and/or based on correlations between levels of expression of the protein and yield. The predicting step also may be carried out, for example, based on differences with respect to the levels of more than one of the proteins.

### Kit for Obtaining a High-Yielding Oil Palm Plant

As noted above, also provided is a kit for obtaining a high-yielding oil palm plant. The kit comprises: (i) an antibody for detection of a protein; and (ii) an extract of a mesocarp tissue of a fruit of a reference oil palm plant. The proteins described above as being useful in the method for obtaining a high-yielding oil palm plant are also useful in the kit for obtaining a high-yielding oil palm plant. Also, as noted above, antibodies to the above-noted proteins may be prepared by methods that are well known in the art or obtained from commercial vendors.

In some embodiments, the kit further comprises instructions indicating use of the antibody for determining whether there is a difference between the level of the protein in mesocarp tissue of a fruit of a parental oil palm plant and the level of the protein in the extract of the mesocarp tissue of the fruit of the reference oil palm plant. The step of determining whether there is such a difference may also be carried as described above. In some embodiments, the kit also further comprises instructions indicating selection of progeny of the parental oil palm plant based on the difference to obtain the high-yielding oil palm plant. The step of selecting progeny of the parental oil palm plant may also be carried out as described above. In addition, in some embodiments, the kit further comprises at least another antibody for detection of at least another of the proteins.

The following examples are for purposes of illustration and are not intended to limit the scope of the claims.

### Example 1

### Two-Dimensional Difference Gel Electrophoresis and Identification of Proteins

### Objectives

Objectives included identifying proteins that are differentially expressed in oil palm mesocarp tissue across high- and low-yielding traits and across time of fruit development.

### Methods

### Screening Populations:

Two screening populations of oil palm plants, a high-yielding screening population and a low-yielding screening population, each consisting of three individual palm plants, were used. The screening populations were derived from crosses of Serdang Avenue dura (at least 75% of Serdang Avenue dura) and AVROS pisifera (at least 75% of AVROS pisifera) to yield tenera progeny. More specifically, the high-yielding screening population was derived from a population of oil palm plants that had previously been determined to yield relatively high amounts of palm oil, specifically more than 10 tonnes of palm oil per hectare per year, and thus to have a high-yielding phenotype, also termed an H phenotype. The low-yielding screening population was derived from a population of oil palm plants that had previously been determined to yield relatively low amounts of palm oil, specifically lower than 6 tonnes of palm per hectare per year, and thus to have a low-yielding phenotype, also termed an h phenotype. The yield determinations for the high-yielding and low-yielding populations were defined by 4-year statistical data collected by reliable oil palm breeders. For the high-yielding screening populations, the three high-yielding palms thereof were designated H2, H4, and H6. For the low-yielding screening population, the three low-yielding palms thereof were designated h1, h6, and h9.

### Comparisons:

Each of the three high-yielding palms and the three low-yielding palms of the screening populations were sampled across three time points, 12 weeks post-pollination (time “a”), 16 weeks post-pollination (time “b”), and 18 weeks post-pollination (time “c”), to provide for the following comparisons:

1. High yield (12 weeks) v. Low yield (12 weeks) (also termed “Ha vs ha”)
2. High yield (16 weeks) v. Low yield (16 weeks) (also termed “Hb vs hb”)
3. High yield (18 weeks) v. Low yield (18 weeks) (also termed “He vs hc”)
4. High yield (12 weeks) v. High yield (16 weeks) (also termed “Ha vs Hb”)
5. High yield (12 weeks) v. High yield (18 weeks) (also termed “Ha vs He”)
6. Low yield (12 weeks) v. Low yield (16 weeks) (also termed “ha vs hb”)
7. Low yield (12 weeks) v. Low yield (18 weeks) (also termed “ha vs hc”)
Specifically, mesocarp tissue was obtained from fruitlets of each of the palms at each of the time points. For reference, oil deposition in the endosperm starts at approximately 12 weeks post-pollination and is almost complete by 16 weeks post-pollination, whereas oil deposition in the mesocarp starts at approximately 15 weeks post-pollination and continues until fruit maturity at about 20 weeks post-pollination. The time points of 12, 16, and 18 weeks post-pollination were chosen because 12 weeks post-pollination marks the start of oil deposition in endosperm but precedes the start of oil deposition in mesocarp, 16 weeks post-pollination marks the point of highest transcript expression level in mesocarp, following the initiation of oil biosynthesis after pollination, and 18 weeks post-pollination marks the time at which transcript expression would be expected to decrease as the fruit matures.

### Preparation of Protein Samples:

Samples of total mesocarp protein were extracted from oil palm fruitlets from each of the three high-yielding palm plants and each of the three low-yielding palm plants at 12, 16, and 18 weeks post-pollination based on, a modified protein extraction method of He et al, 7 Forestry Studies in China 20, 20-23 (2005). The protein samples were resuspended in 2-D cell lysis buffer (30 mM Tris-HCl, pH 8.8, containing 7 M urea, 2M thiourea and 4% CHAPS). The mixture was sonicated at 4° C. followed by shaking for 30 minutes at room temperature. The samples were centrifuged for 30 minutes at 14,000 rpm and the supernatant was collected. Protein concentration of the supernatant fraction was measured using Bio-Rad protein assay method (Bradford, 1976).

### Internal Standard:

An internal standard was made by mixing an equal amount of protein from each sample, i.e. total mesocarp protein from each of the three high-yielding palms and three low-yielding palms, at each of the three time points of 12, 16, and 18 weeks post-pollination. The internal standard was used to match and normalize protein patterns across different gels, thereby negating the problem of inter-gel variation. This approach allowed accurate quantification of differences between samples with an associated statistical significance. Quantitative comparisons of protein between samples were made based on the relative change of each protein spot to its own in-gel internal standard.

### CyDye Labeling:

For each sample, 30 μg of protein was mixed with 1.0 μl of diluted CyDye, and kept in dark on ice for 30 min. Samples from each pairwise comparison were labeled with Cy3 and Cy5 respectively. The internal standard was labeled with Cy2. The labeling reaction was stopped by adding 1.0 μl of 10 mM lysine to each sample, and incubating in dark on ice for an additional 15 min. The three labeled samples were then mixed together. The 2×2-D Sample buffer (8 M urea, 4% CHAPS, 20 mg/ml DTT, 2% pharmalytes and trace amount of bromophenol blue), 100 μl de-streak solution and rehydration buffer (7 M urea, 2 M thiourea, 4% CHAPS, 20 mg/ml DTT, 1% pharmalytes and trace amount of bromophenol blue) were added to the labeling mix to make the total volume of 250 μl. Samples were mixed well and spun down. Then samples were loaded onto immobilized pH gradient gel (“IPG”) strips housed in a strip holder.

### Difference Gel Electrophoresis Gels:

Difference gel electrophoresis (“DIGE”) analytical gels were designed to contain the appropriate sample pairings in order to facilitate gel analysis in the later part of the experiment. A total of nine DIGE gels were produced with the sample pairings as follow:

Gel 1: H2a, h1a, and internal standard
Gel 2: H4a, h6a, and internal standard
Gel 3: H6a, h9a, and internal standard
Gel 4: H2b, h1b, and internal standard
Gel 5: H4b, h6b, and internal standard
Gel 6: H6b, h9b, and internal standard
Gel 7: H2c, h1c, and internal standard Gel 8: H4c, h6c, and internal standard
Gel 9: H6c, h9c, and internal standard
Accordingly, gels 1-3, 4-6, and 7-9 correspond to comparisons of high-versus low-yielding palms at 12, 16, and 18 weeks post-pollination, respectively.

### Isoelectric Focusing and SDS-Polyacrylamide Gel Electrophoresis:

After loading the labeled samples onto IPG strips of pH 4-7, isoelectric focusing (“IEF”) was conducted following a known protocol of Amersham BioSciences, 2-D Electrophoresis: Principles and Methods, pp. 43-72 (2004). Upon finishing the IEF, the IPG strips were incubated in freshly made equilibration buffer-1 (50 mM Tris-HCl, pH 8.8, containing 6 M urea, 30% glycerol, 2% SDS, trace amount of bromophenol blue and 10 mg/ml DTT) for 15 minutes with gentle shaking. Then the IPG strips were rinsed in the freshly made equilibration buffer-2 (50 mM Tris-HCl, pH 8.8, containing 6 M urea, 30% glycerol, 2% SDS, trace amount of bromophenol blue and 45 mg/ml DTT) for 10 minutes with gentle shaking. The IPG strips were rinsed in the SDS-polyacrylamide gel electrophoresis (“SDS-PAGE”) gel running buffer and then transferred into 12% SDS-PAGE gels. The SDS-PAGE gels were run at 15° C. until the dye front ran out of the gels.

### Image Scan and Data Analysis:

Gel images were scanned immediately following SDS-PAGE using Typhoon TRIO (Amersham BioSciences) in accordance with known methods for use thereof. The scanned images were then analyzed by Image Quant software (version 6.0, Amersham BioSciences), followed by Biological Variation Analysis (“BVA”) using DeCyder™ 2D software version 6.5 (Amersham BioSciences).

### Results

Results from 2D-DIGE Analytical Gels:

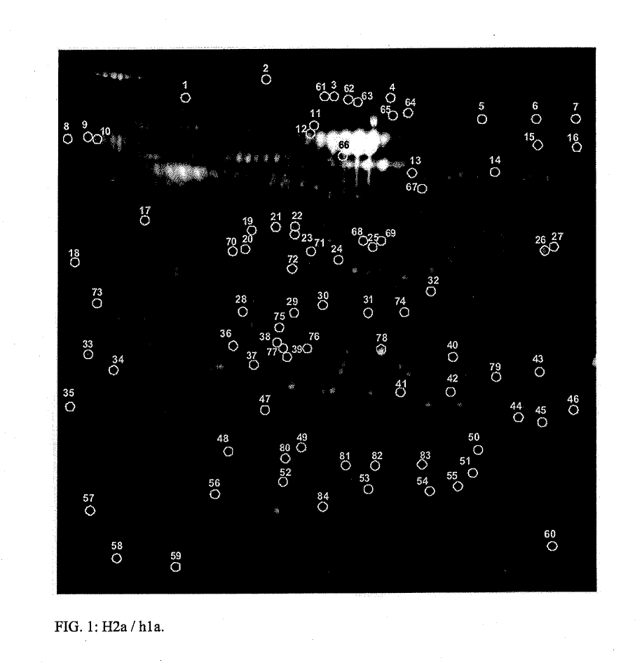

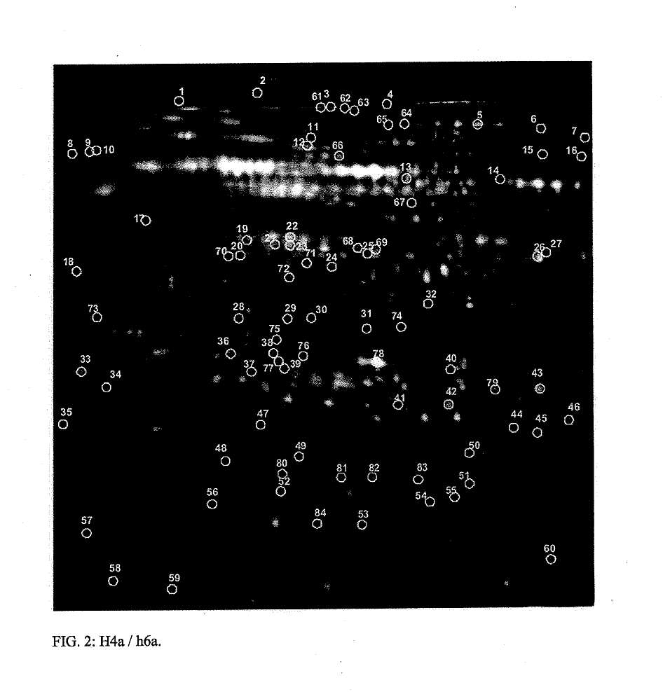

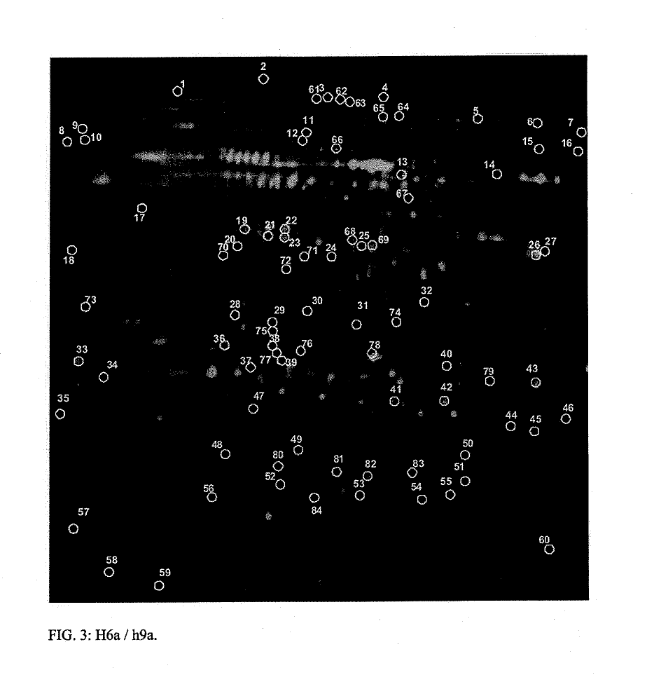

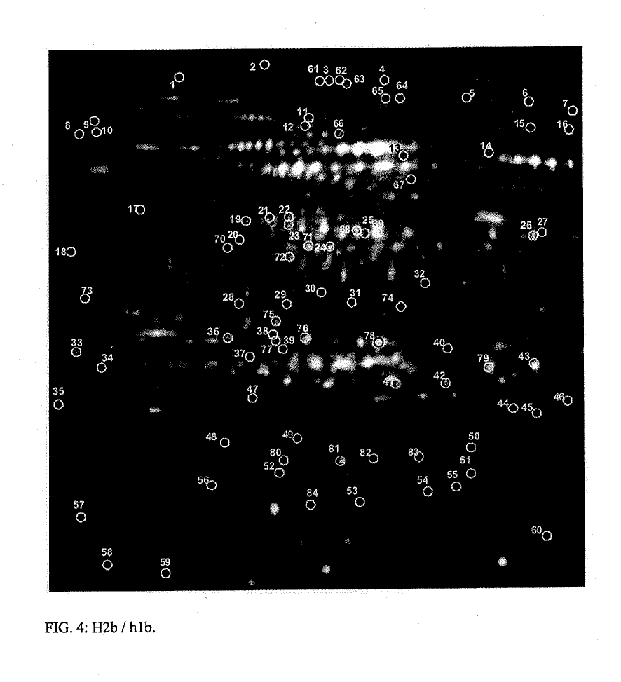

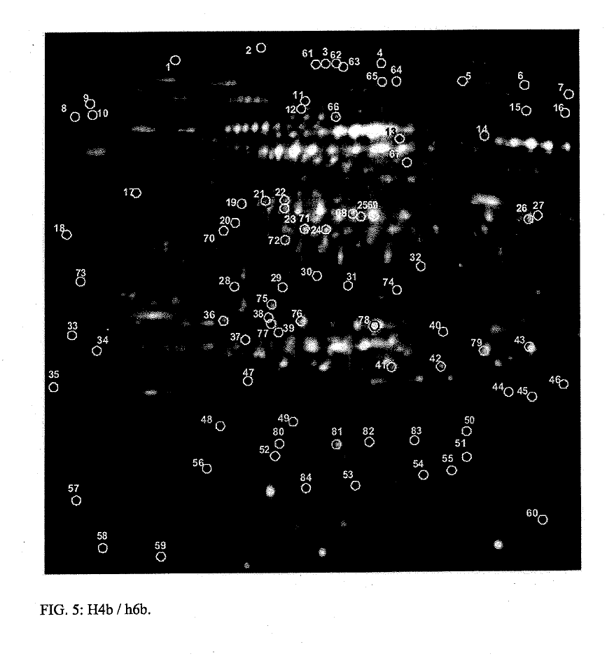

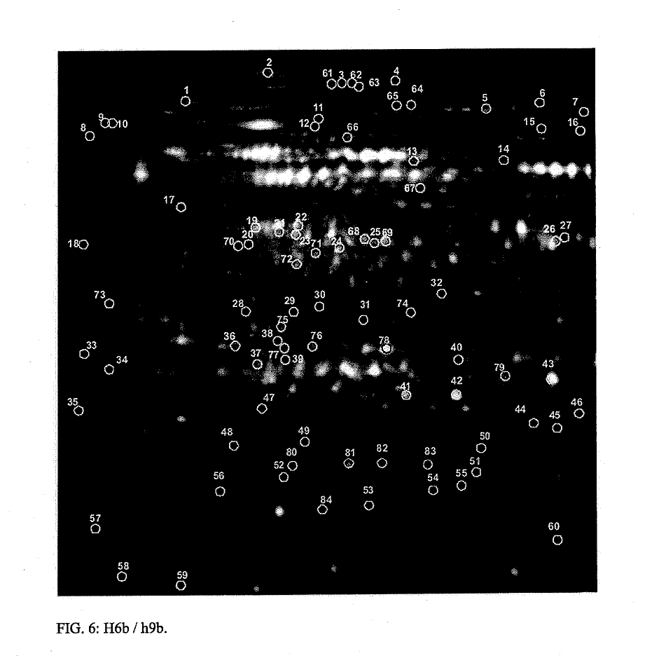

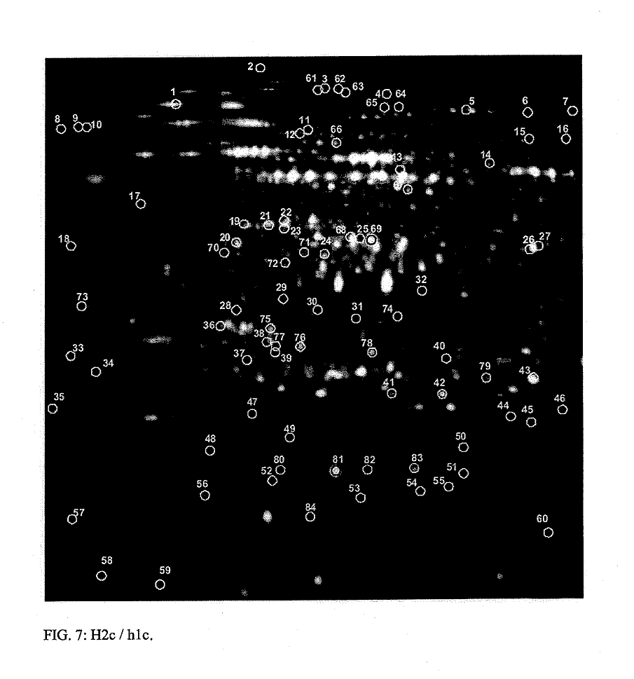

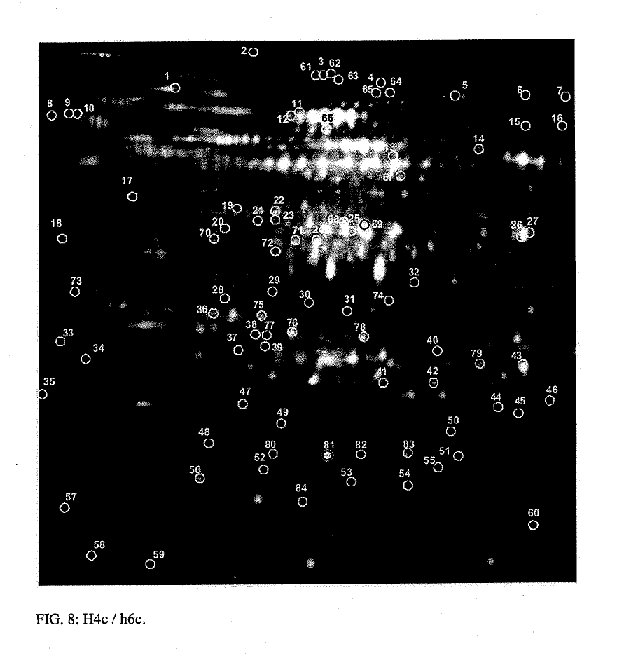

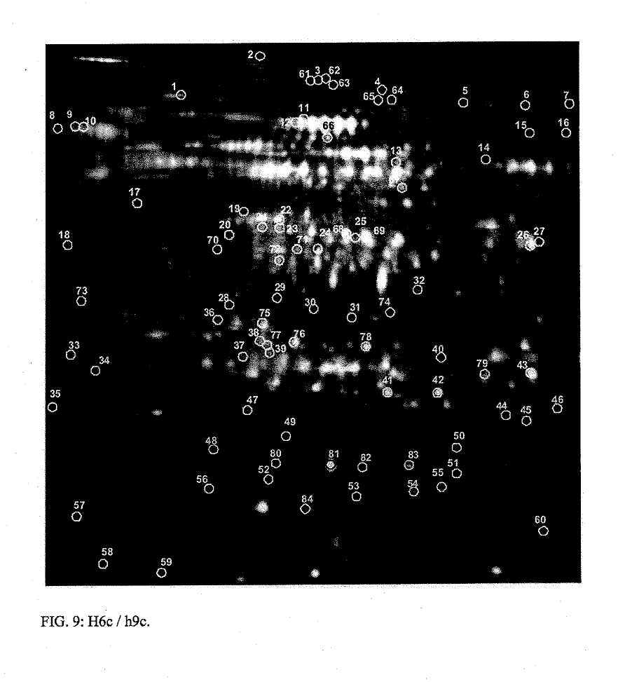

Results for 2D-DIGE analytical gels are shown in FIGS. 1-9, corresponding to comparisons of (1) H2a vs. h1a, (2) H4a vs. h6a, (3) H6a vs. h9a, (4) H2b vs. h1b, (5) H4b vs. h6b, (6) H6b vs. h9b, (7) H2c vs. h1c, (8) H4c vs. h6c, and (9) H6c vs. h9c, respectively.

The DIGE analytical gels were used for cross-gel BVA analysis as follows:

i) Hb vs hb, corresponding to (H2b, H4b, H6b) vs (h1b, h6b, h9b)
ii) Ha vs ha, corresponding to (H2a, H4a, H6a) vs. (h1a, h6a, h9a)
iii) Hc vs hc, corresponding to (H2c, H4c, H6c) vs. (h1c, h6c, h9c)
iv) Ha vs Hb vs Hc, corresponding to (H2a, H4a, H6a) vs. (H2b, H4b, H6b) vs. (H2c, H4c, H6c)
v) ha vs hb vs hc, corresponding to (h1a, h6a, h9a) vs. (h1b, h6b, h9b) vs. (h1c, h6c, h9c)

Based on matching multiple gels for comparison and conducting statistical analysis of protein-abundance changes, the BVA analysis provides an accurate indication of the differential expression of protein spots. For purposes of determining statistical significance in this analysis, p value<0.1 was applied due to the small sample size (n=3) for each gel-to-gel comparison. The number of differentially expressed proteins found in this analysis was narrowed down to those with expression ratios of >1.5 fold change. The corresponding protein spots in the gels were cross-checked by eye to ensure good quality/resolution spots with no artificial streaks. The screened spots were subsequently picked for identification via mass spectrometry (“MS”).

In accordance with these methods, 84 protein spots were detected as differentially expressed from 2D-DIGE BVA analysis in one or more of the above-noted analyses, as shown as in FIGS. 1-9 as protein spots that are circled and numbered.

The 84 spots were narrowed down further to 61 spots for mass spectrometry identification via MALDI-ToF/ToF. The selection criteria practiced here was based on i) visibility of the spots on the DIGE gel, ii) differences in protein expression greater than 1.5 fold, and iii) the occurrence of protein isoforms. Candidate spots that were not clearly visible on the DIGE gel, that had lower than 1.5 fold change in expression, or that were redundant based on an isoform thereof having been identified already, were not selected for further analysis.

### 2D-DIGE Preparative Gels:

Three preparative gels were run with 3 CyDye labeling. Samples of total protein from individual palms were mixed for identification purposes, because individual samples did not include sufficient protein. Samples selected for preparative gels were as follows:

Gel 1: H4a, h6a, mix of other Ha/ha
Gel 2: H4b, h6b, mix of other Hb/hb
Gel 3: H2c, h1c, mix of other Hc/hc

The preparative gels were used for spot-picking all of the 61 protein spots of interest. For each picked spot, the identity of the corresponding protein was determined by MS. Specifically, each protein was subjected to MALDI-ToF/ToF, amino acid sequences of peptide fragments thereof were determined, the sequences were compared to the NCBI non-redundant database for identification of the nearest homolog, and an identity was assigned to the protein based on the identity of the nearest homolog.

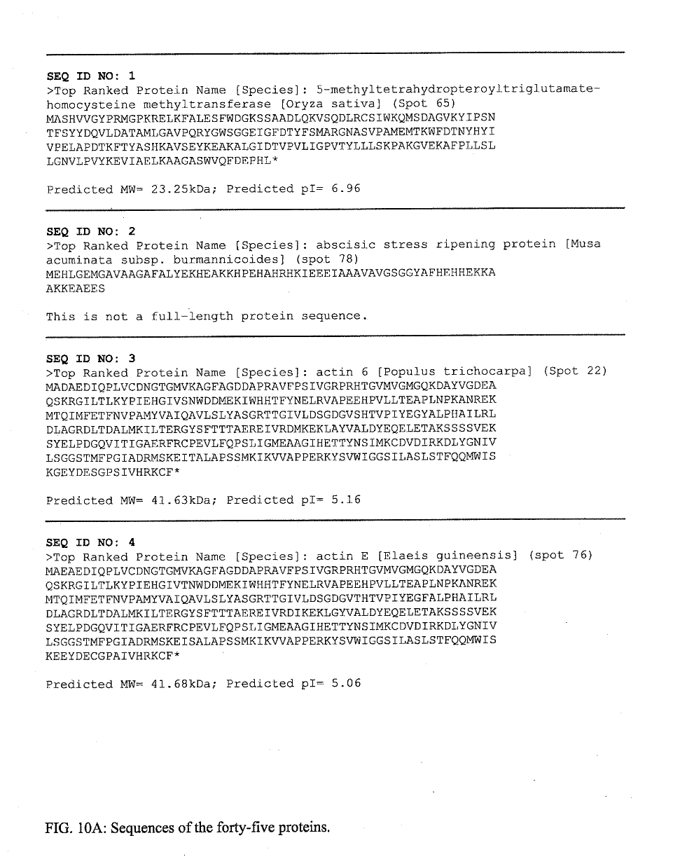

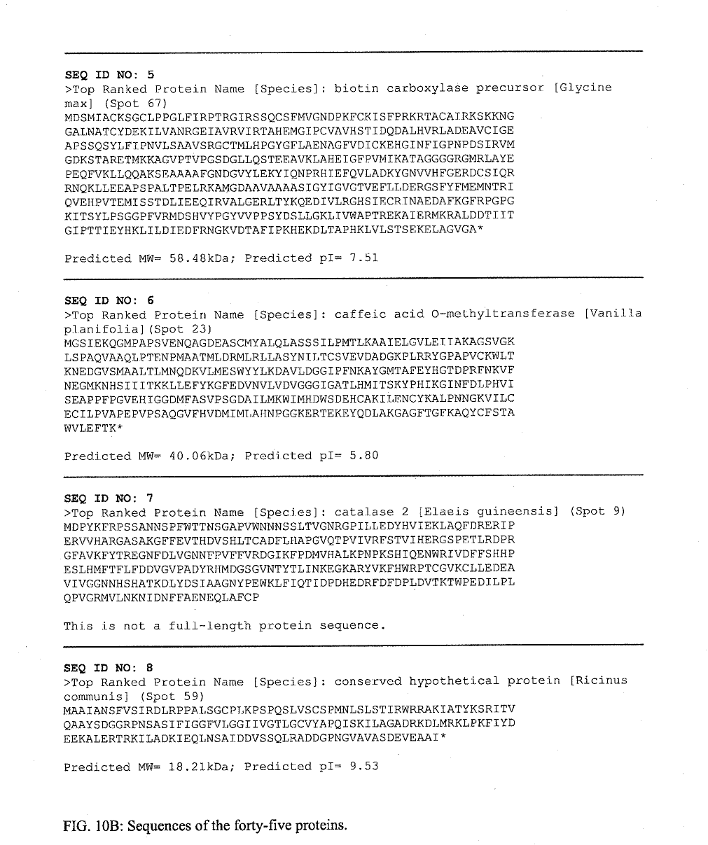

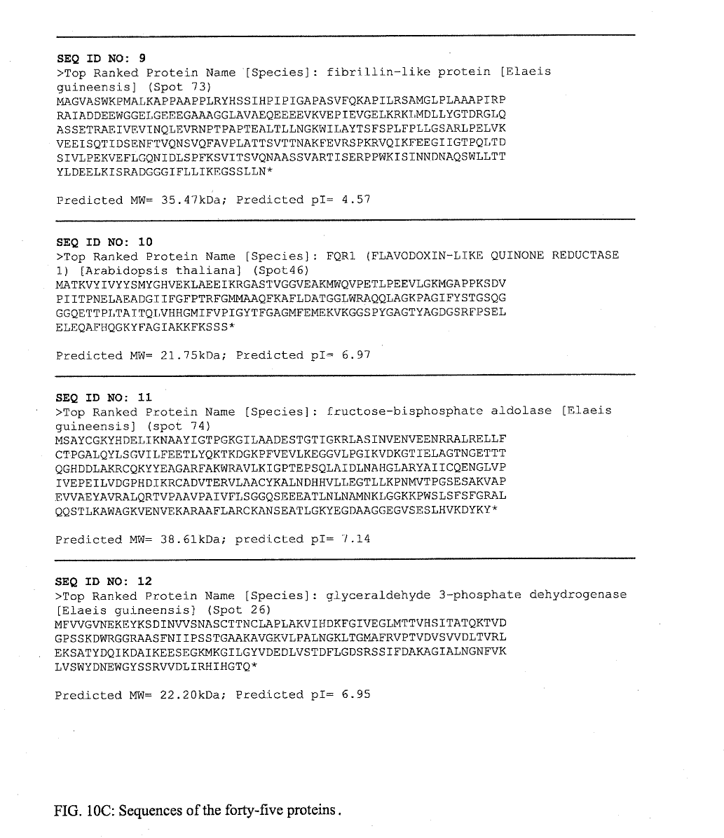

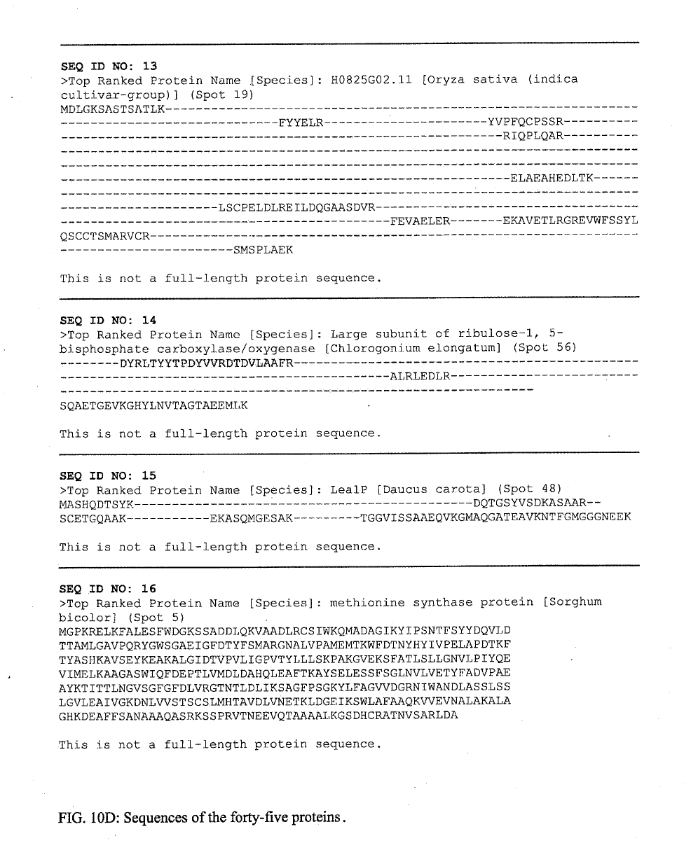

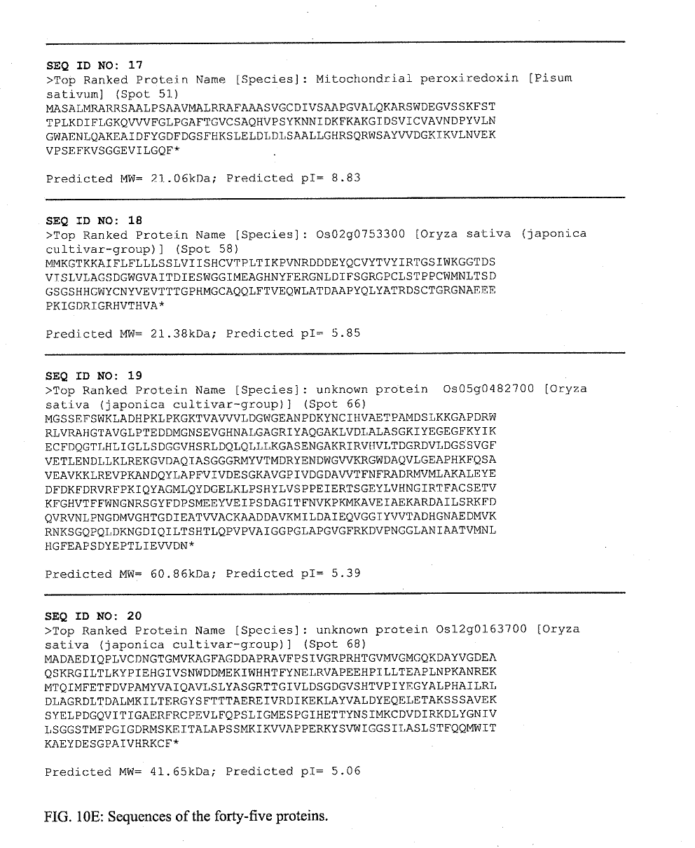

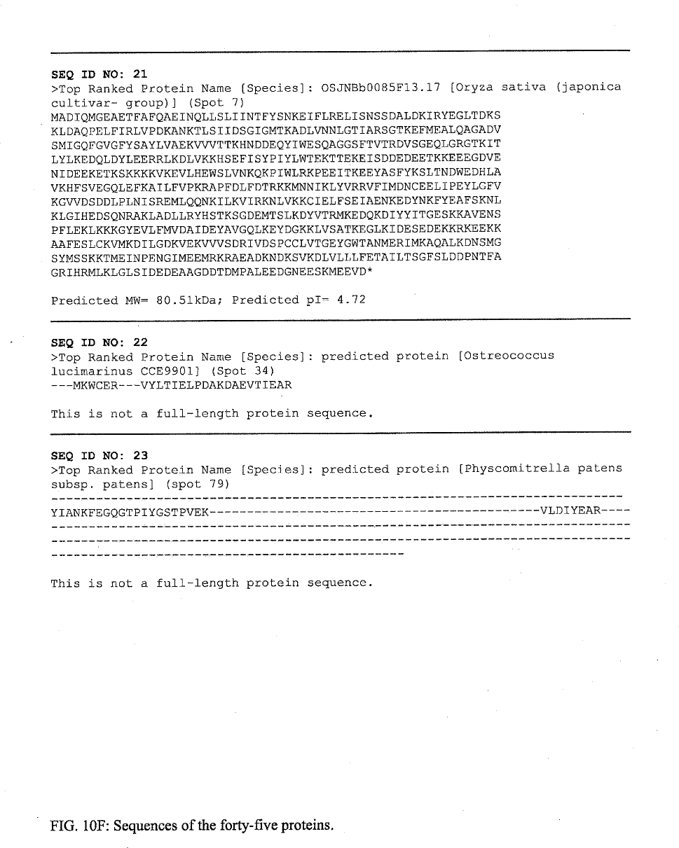

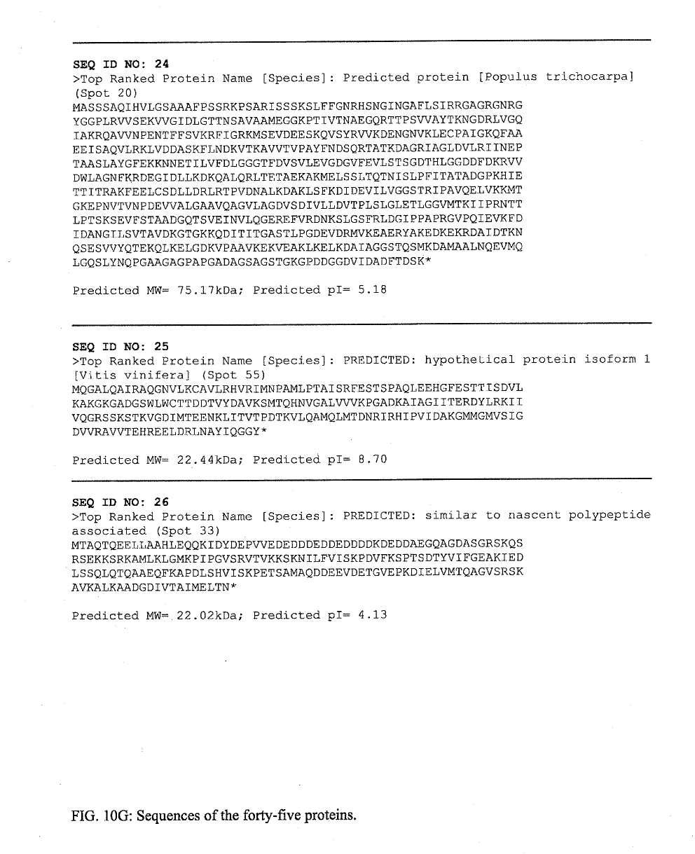

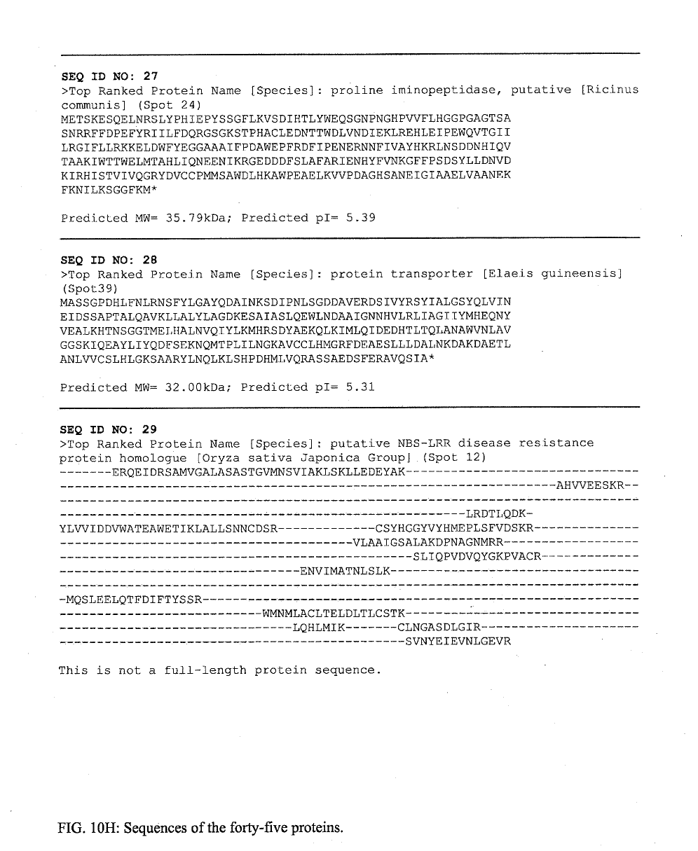

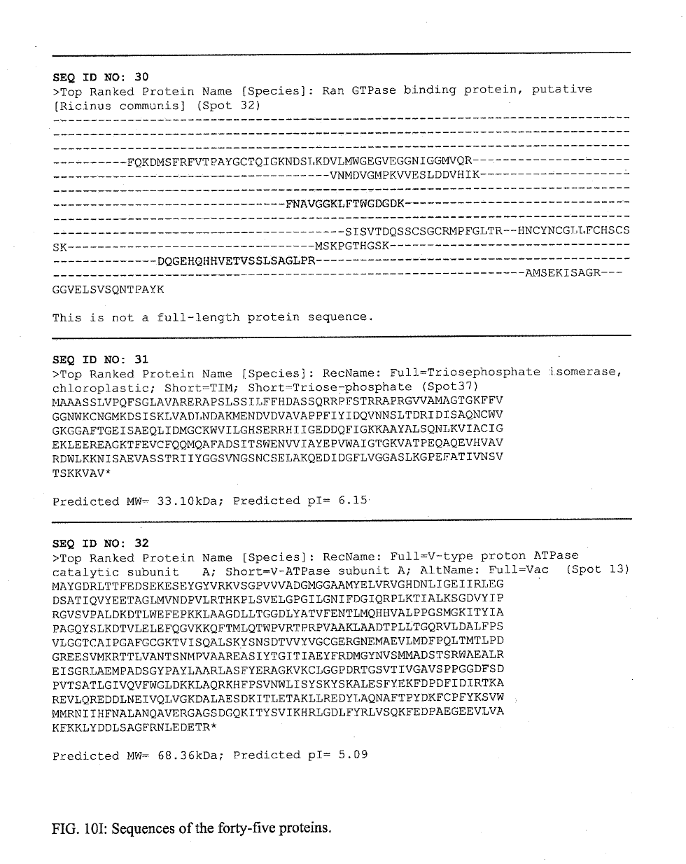

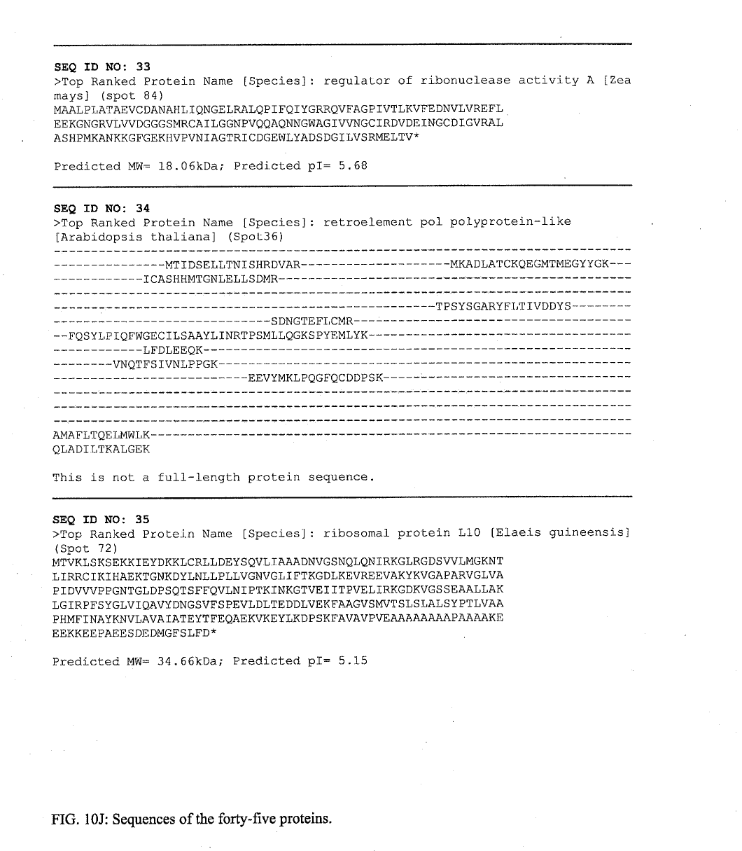

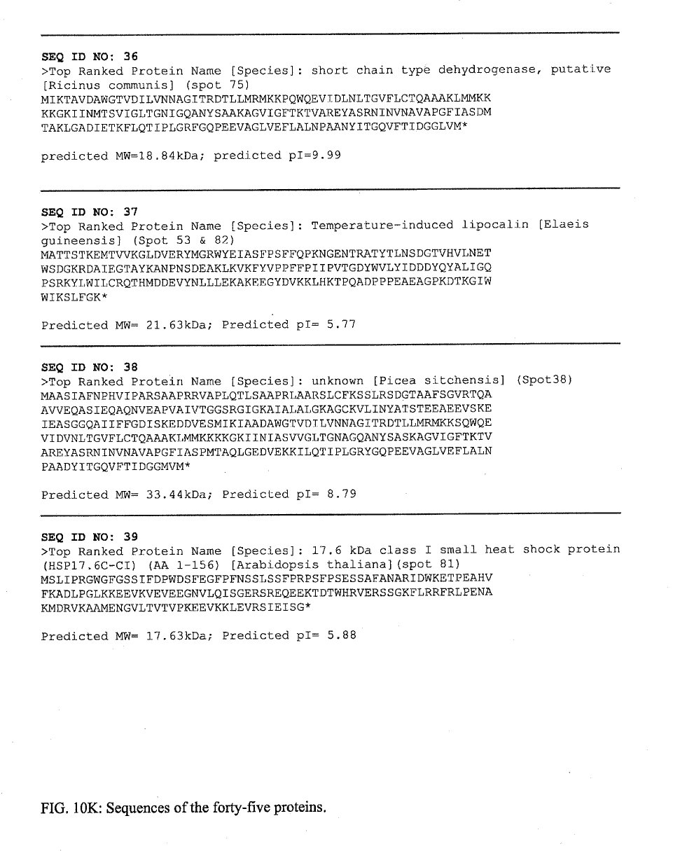

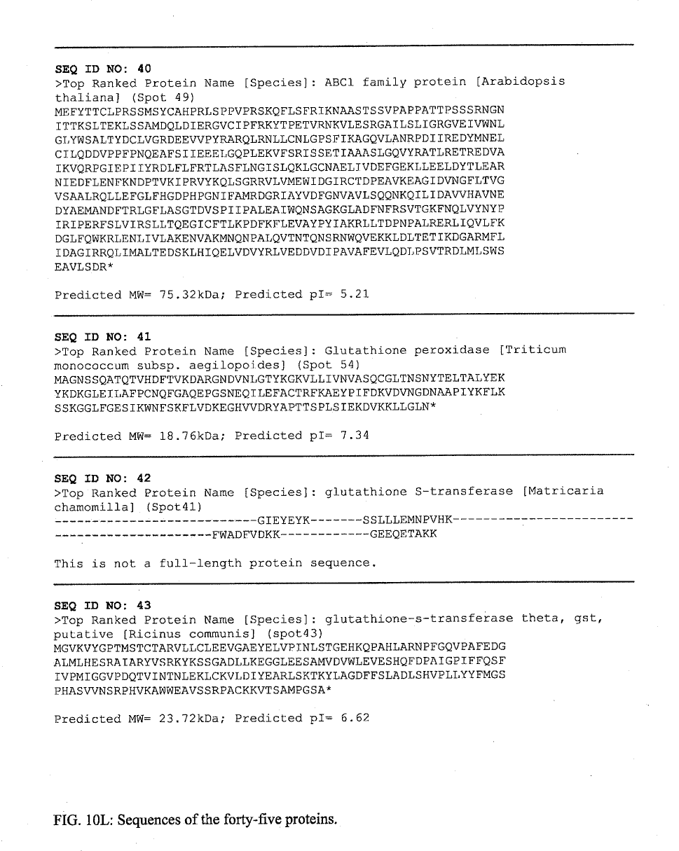

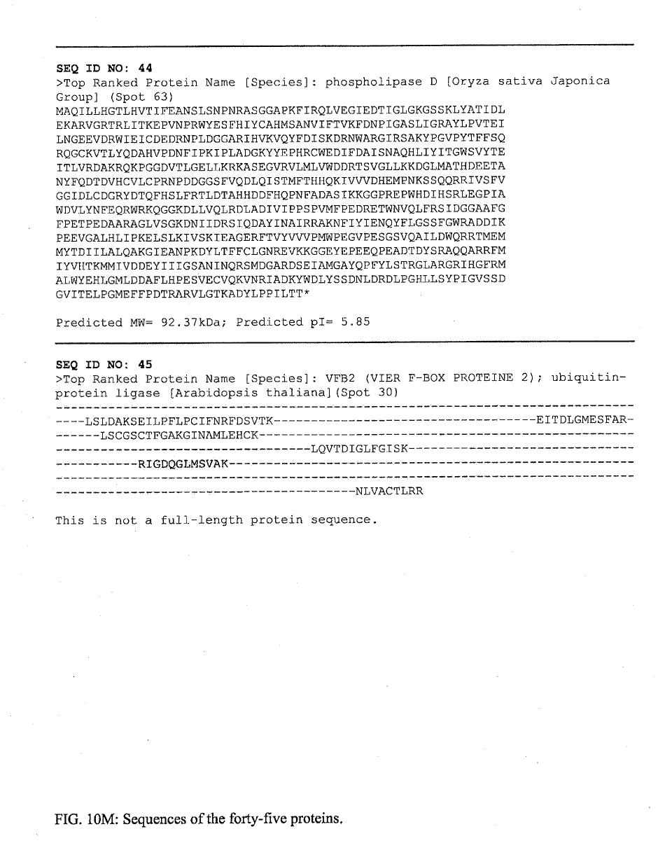

This analysis resulted in 45 unique protein identifications corresponding to oil palm proteins that are homologous to proteins previously identified in oil palm or other organisms, as shown in TABLE 1, and that are differentially expressed with respect to high- or low-yielding palms and/or across the time points of 12, 16, and 18 weeks post-pollination, as shown in TABLE 2, and that thus are related to high/low yielding traits in oil palm. Specifically, of the 61 protein spots, 8 yielded matches of no confidence, i.e. the confidence index scores for the matches between each of the 8 protein spots and the nearest homologous proteins in the NCBI non-redundant database were below 80%. These 8 protein spots were not considered further. Moreover, 8 protein spots yielded repetitive identifications, i.e. the identifications were redundant with respect to other protein spots. These 8 protein spots also were not considered further. The remaining 45 unique protein identifications correspond to the following oil palm proteins: 5-methyltetrahydropteroyltriglutamate-homocysteine methyltransferase comprising SEQ ID NO: 1, abscisic stress ripening protein comprising SEQ ID NO: 2, actin 6 comprising SEQ ID NO: 3, actin E comprising SEQ ID NO: 4, biotin carboxylase precursor comprising SEQ ID NO: 5, caffeic acid O-methyltransferase comprising SEQ ID NO: 6, catalase 2 comprising SEQ ID NO: 7, conserved-hypothetical-protein-of-Ricinus-communis ortholog comprising SEQ ID NO: 8, fibrillin-like protein comprising SEQ ID NO: 9, flavodoxin-like quinone reductase 1 comprising SEQ ID NO: 10, fructose-bisphosphate aldolase comprising SEQ ID NO: 11, glyceraldehyde 3-phosphate dehydrogenase comprising SEQ ID NO: 12, H0825G02.11 ortholog comprising SEQ ID NO: 13, large subunit of ribulose-1,5-bisphosphate carboxylase/oxygenase comprising SEQ ID NO: 14, LealP comprising SEQ ID NO: 15, methionine synthase protein comprising SEQ ID NO: 16, mitochondrial peroxiredoxin comprising SEQ ID NO: 17, Os02g0753300 ortholog comprising SEQ ID NO: 18, Os05g0482700 ortholog comprising SEQ ID NO: 19, Os12g0163700 ortholog comprising SEQ ID NO: 20, OSJNBb0085F13.17 ortholog comprising SEQ ID NO: 21, predicted-protein-of-Ostreococcus-lucimarinus-CCE9901 ortholog comprising SEQ ID NO: 22, predicted-protein-of-Physcomitrella patens-subsp.-patens ortholog comprising SEQ ID NO: 23, predicted-protein-of-Populus-trichocarpa ortholog comprising SEQ ID NO: 24, hypothetical-protein-isoform-1-of-Vitis-vinifera ortholog comprising SEQ ID NO: 25, nascent polypeptide associated complex alpha comprising SEQ ID NO: 26, proline iminopeptidase comprising SEQ ID NO: 27, protein transporter comprising SEQ ID NO: 28, putative-NBS-LRR-disease-resistance-protein-homologue-of-Oryza-sativa-Japonica-Group ortholog comprising SEQ ID NO: 29, Ran GTPase binding protein comprising SEQ ID NO: 30, chloroplastic triosephosphate isomerase comprising SEQ ID NO: 31, V-type proton ATPase catalytic subunit A comprising SEQ ID NO: 32, regulator of ribonuclease activity A comprising SEQ ID NO: 33, retroelement pol polyprotein-like ortholog comprising SEQ ID NO: 34, ribosomal protein L10 comprising SEQ ID NO: 35, short chain type dehydrogenase comprising SEQ ID NO: 36, temperature-induced lipocalin comprising SEQ ID NO: 37, unknown-protein-of-Picea-sitchensis ortholog comprising SEQ ID NO: 38, 17.6 kDa class I small heat shock protein comprising SEQ ID NO: 39, ABC1 family protein comprising SEQ ID NO: 40, glutathione peroxidase comprising SEQ ID NO: 41, glutathione S-transferase comprising SEQ ID NO: 42, glutathione-S-transferase theta comprising SEQ ID NO: 43, phospholipase D comprising SEQ ID NO: 44, and VIER F-Box Proteine 2 comprising SEQ ID NO: 45. The sequences of the 45 proteins are provided in FIG. 10A-M. Of note, SEQ ID NOs: 1, 3-6, 8-12, 17-21, 24-28, 31-33, 35-41, 43, and 44 correspond to amino acid sequences of full length proteins, as deduced by determining nucleotide sequences of the corresponding mRNA transcripts, which themselves were identified based on amino acid sequences of various non-consecutive peptide fragments of the proteins as determined by MS. In contrast, SEQ ID NOs: 2, 7, 13-16, 22, 23, 29, 30, 34, 42, and 45 correspond to non-full length protein sequences, i.e. the N- and or C-terminal sequences of the corresponding full length protein have not been determined, or amino acid sequences of various non-consecutive peptide fragments of the proteins as determined by MS.

### Example 2

### Functions of the 45 Unique Differentially Expressed Proteins

The 45 unique differentially expressed proteins identified in. Example 1 were annotated based on predicted molecular function, pathway involvement and enzyme classification, as shown in TABLE 1. Surprisingly, of the 45 proteins, only three are functionally related to lipid metabolism. The three proteins are phospholipase D, biotin carboxylase precursor, and fructose-bisphosphate aldolase. Moreover, only 17 have been successfully mapped to so-called KEGG Pathways, i.e. pathways for which the proteins thereof play a role in metabolism of carbohydrates, amino acids, lipids, nucleotides, energy, or secondary metabolites, in accordance with the KEGG (Kyoto Encyclopedia of Genes and Genomes) pathway database, available at: http://www.genome.jp/keg/pathway,html (last accessed November 2010). The 17 proteins include the three above-noted lipid metabolism proteins and 5-methyltetrahydropteroyltriglutamate-homocysteine methyltransferase, caffeic acid O-methyltransferase, catalase 2, glyceraldehyde 3-phosphate dehydrogenase, large subunit of ribulose-1,5-bisphosphate carboxylase/oxygenase, methionine synthase protein, proline iminopeptidase, Ran GTPase binding protein, chloroplastic triosephosphate isomerase, V-type proton ATPase catalytic subunit A, glutathione peroxidase, glutathione S-transferase, glutathione-S-transferase theta, and VIER F-Box Proteine 2. The remaining 28 differentially expressed proteins were not known to be involved in oil biosynthesis. The remaining proteins include abscisic stress ripening protein, actin 6, actin E, conserved-hypothetical-protein-of-Ricinus-communis ortholog, fibrillin-like protein, flavodoxin-like quinone reductase 1, H0825G02.11 ortholog, LealP, mitochondrial peroxiredoxin, Os02g0753300 ortholog, Os05g0482700 ortholog, Os12g0163700 ortholog, OSJNBb0085F13.17 ortholog, predicted-protein-of-Ostreococcus-lucimarinus-CCE9901 ortholog, predicted-protein-of-Physcomitrella patens-subsp.-patens ortholog, predicted-protein-of-Populus-trichocarpa ortholog, hypothetical-protein-isoform-1-of-Vitis-vinifera ortholog, nascent polypeptide associated complex alpha, protein transporter, putative-NBS-LRR-disease-resistance-protein-homologue-of-Oryza-sativa-Japonica-Group ortholog, regulator of ribonuclease activity A, retroelement pol polyprotein-like ortholog, ribosomal protein L10, short chain type dehydrogenase, temperature-induced lipocalin, unknown-protein-of-Picea-sitchensis ortholog, 17.6 kDa class I small heat shock protein, and ABC1 family protein.

### Example 3

### Dot Blot-Based Detection of Various of the 45 Unique Differentially Expressed Proteins Objectives

Objectives included validating protein leads obtained from the DIGE experiment of Example 1 in larger populations of high and low yielding oil palms.

### Methods

### Samples:

Mesocarp tissues of 8 high and 8 low yielding palms obtained across 6 time points, namely 12, 14, 16, 18, 20 and 22 weeks post-pollination, were used.

### Antibodies:

Antibodies against 27 of the 45 unique differentially expressed proteins were obtained from various suppliers, as indicated in Table 3.

### Protein Extraction (TCA Extraction):

TCA extraction buffer (containing 10% TCA (10 g), 0.007% DTT (70 mg), and acetone (to 100 ml) and stored at −20° C.) pre-cooled at −20° C. (0.2 g+0.5 ml) was added to mesocarp samples in the form of fine powder. Samples were ground further using a mini plastic grinder. Samples were mixed and mashed well. Then another 1 ml of buffer was added and samples were incubated at −20° C. for 1 hour. Samples were then subjected to centrifugation at maximum speed (13.2 g) at 4° C. for 15 min. Tubes were placed on ice and supernatant was removed by use of a pipette. A volume of 1.8 ml wash buffer (containing 0.007% DTT (70 mg) and acetone (to 100 ml) and stored at −20° C.) was added and pellets were resuspended and crushed by use of pipette tips. Samples were incubated at −20° C. for 1 hour. Samples were again subjected to centrifugation at maximum speed at 4° C. for 15 min. Supernatant was removed and washing steps were repeated for a total of three times. Sample powders were air-dried on ice for 30 min. Dried sample powders were then resuspended in 500 μl of lysis/USB buffer (containing 9M urea (5.4 g), 4% CHAPS (0.4 g), 1% DTT (0.1 g), 1% ampholytes pH 3-10 (250 μl), 35 mM Tris Base (0.0424 g), sterile MilliQ water (to 10 ml), all filtered through 0.2 μm pore size membrane and stored at −20° C.). Samples were incubated at 37° C. for 1 hour with continuous shaking. Samples were subjected to centrifugation at max speed at room temperature for 15 min. Supernatants were transferred to clean tubes and stored at −80° C. Pellets were stored at −80° C. as back-up for further use. To further elute protein from pellet, an additional 500 μl of lysis/USB buffer can be added, followed by incubation of the pellet at room temperature for 1 hour with shaking, transfer of the back-up supernatants to clean microcentrifuge tubes, and finally storage at −80° C.

### Protein Quantification (Bradford Assay):

Samples corresponding to 5-fold dilutions of protein stocks were prepared for quantification. The BSA stock concentration was 1.4 μg/μl. Six points of 2-fold serial dilutions were used to construct the standard curve. Concentrations obtained for samples ranged from 0.244 μg/μl (lowest) to 2.934 μg/μl (highest). Having determined the concentrations of protein stocks, working stocks (330 μl) at a final concentration of 0.2 μg/μl were prepared by use of PBS buffer (+10% glycerol) for dot-blotting onto membrane.

### Dot-Blot Screening:

### Dot-Blot Arrays Using a 386 Pin Replicator:

For each blot, a nitrocellulose membrane was cut to 2.95×4.6 inches and pasted onto single well plates. A plate was stacked with membrane on top of another empty plate and then the stamping guide was stacked over both plates. Protein samples were prepared in two concentrations, 0.20 μg/μl and 0.02 μg/μl (10× dilution). The replicator was dipped into the 386-well plate and swirled. The replicator was lifted and the guiding pins were slotted into the guide slots on the stamping guide. The blot was then stamped and fan dried. When the membrane was fully dried, the stamping procedure was repeated for a total of 5 rounds (equivalent to stamping 0.20 μg or 0.02 μg of protein on each spot since replicator pins delivers 0.2 μl of sample), with the membrane being fan dried after each application. Membranes were then allowed to air dry overnight. Membranes were removed from plates and cut down to size. The membranes were kept sandwiched between the original protective paper and stored in air-tight containers in a dry environment until use.

### Preparation for Screening:

Individual membranes were clipped onto glass slides, two on each slide, with their backs facing inward. The clipped membranes were dipped into a container filled with cold 0.1% PBS-T (pH 7.4) and stirred at about speed 7 on a magnetic stirrer for 40 minutes. The 0.1% PBS-T was replaced with cold 0.05% PBS-T and washing was continued for 15 minutes. The 0.05% PBS-T was replaced with fresh cold 0.05% PBS-T, stirring was continued for 7 minutes, and then the replacement and stirring steps were repeated.

### Antibody Incubations:

Membranes were laid in clean and appropriately sized incubation containers, and any bubbles trapped underneath the membranes were removed. A volume of 1 ml of PBS-T 0.05% was pipetted onto blank membranes and 1 ml antibody was diluted in PBS-T 0.05% onto corresponding membranes. The membranes were shaken to ensure that the whole surface of membranes was covered. Containers were covered with wet c-fold towels before wrapping with cling-film to keep in moisture. The membranes were then incubated overnight on a Belly Dancer laboratory shaker at 4° C. or at room temperature for 2 to 3 hours depending on optimized conditions for individual antibodies. Used sera were retained for further experiments or discarded into a bottle to be autoclaved. Membranes were clipped onto glass slides. Membranes were washed in cold 0.05% PBS-T for 15 min, and then were twice washed in fresh 0.05% PBS-T for 7 min each time. Membranes were laid back into clean incubation containers to ensure that no bubbles were trapped underneath the membranes. A volume of 1 ml of secondary antibody diluted in 0.05% PBS-T was added to each membrane. For secondary antibodies with background signals, pre-adsorption was performed with 1% BSA with shaking at room temperature for 40 min. Containers were covered in a similar pattern as above and incubated for 2.5 hours on a Belly Dancer laboratory shaker at room temperature. Secondary antibody was discarded and then the above-described washing steps were repeated for 1.5 min, 7 min, and 7 min, each round with fresh, cold 0.05% PBS-T.

### Development and Documentation:

Membranes were laid back in incubation containers, it was ensured that no bubbles were trapped underneath the membranes, and any remaining 0.05% PBS-T was flicked off of the membranes. Fresh NBT/BCIP was prepared according to manufacturer guidelines using alkaline phosphatase (AP) buffer (100 mM Tris [pH 9.0], 150 mM NaCl, 1 mM MgCl2). A volume of 1.5 ml of NBT/BCIP solution was added to each membrane, followed by incubation on a Belly Dancer laboratory shaker until the purple color of the positive controls was well developed (approximately 30-45 min). The reactions were then stopped by rinsing and soaking membranes in water. The developed membranes were scanned by use of an HP paper scanner (while the membrane remained wet) with HP Director software and the resulting images were saved in TIFF format. Settings were as follows: (a) Highlights: 255; (b) Shadows: 50; (c) Midtones: 2.00; (d) Sharpen: Medium; (e) Resolution: 150; and (f) White Level: 240. Scanned images were further processed by use of Adobe Photoshop C84 Extended & Olympus Micro software to automatically capture and transform spot densities into Microsoft Excel spreadsheets. Data generated from dot-blot immunoassays were analyzed using the Mann Whitney statistical test.

### Results:

Result of the dot-blot immunoassays indicated significant differences in the expression of 11 out of the 27 proteins assayed, as shown in TABLE 4. Specifically, in mesocarp tested 12 weeks post-pollination the proteins caffeic acid O-methyltransferase, chloroplastic triosephosphate isomerase, ABC1 family protein, nascent polypeptide-associated complex alpha, glutathione peroxidase and fructose-bisphosphate aldolase were found to be differentially expressed. In mesocarp tested 14 weeks post-pollination, nascent polypeptide-associated complex alpha and ribosomal L10 proteins were found to be differentially expressed between high and low yielding palms. In mesocarp tested 16 weeks post-pollination, chloroplastic triosephosphate isomerase was differentially expressed. In mesocarp tested 20 weeks post-pollination, glutathione-S-transferase theta and predicted-protein-of-Physcomitrella patens-subsp.-patens ortholog were differentially expressed in mesocarps of high and low yielders. And in mesocarp tested 22 weeks post-pollination, large subunit of ribulose-1,5-bisphosphate carboxylase/oxygenase protein and 17.6 kDa class I small heat shock protein were differentially expressed between high and low yielding palms.

### INDUSTRIAL APPLICABILITY

The methods and kits disclosed herein are useful for obtaining high-yielding oil palms and for predicting oil yields of test oil palm plants, and thus for improving commercial production of palm oil.

TABLE 1
Forty-five unique differentially expressed proteins.

| Pro- | Spot | Top Ranked Protein Name | | | Pathways/ | |
|---|---|---|---|---|---|---|
| tein | # | [Species] | Accession # | Function | Metabolism | Enzyme Class |
| SEQ ID | 65 | 5-methyltetrahydropteroyl- | gi\|108862992 | Catalyzes | Cysteine and | EC2.1.1.14 5-- |
| NO: 1 | | triglutamate-homocysteine methyl- | | methionine | methionine | methyltetrahydropteroyl- |
| | | transferase, putative, expressed | | formation in | metabolism; | triglutamate-homocysteine |
| | | [Oryza sativa] | | amino-acid | biosynthesis of | methyltransferase |
| | | | | biosynthesis | plant hormones | |
| | | | | pathway | | |
| SEQ ID | 78 | abscisic stress ripening protein [Musa | gi\|219963178 | Stress related | — | — |
| NO: 2 | | acuminata subsp. burmannicoides] | | protein | | |
| SEQ ID | 22 | actin 6 [Populus trichocarpa] | gi\|224117708 | Housekeeping, | — | — |
| NO: 3 | | | | monomeric | | |
| | | | | subunit of | | |
| | | | | microfilaments | | |
| SEQ ID | 76 | actin E [Elaeis guincensis] | gi\|48527433 | Actin | — | — |
| NO: 4 | | | | | | |
| SEQ ID | 67 | biotin carboxylase precursor [Glycine | gi\|3219361 | Component of | Fatty acid | EC6.3.4.14 biotin |
| NO: 5 | | max] | | acetyl CoA | biosynthesis; | carboxylase |
| | | | | carboxylase & | pyruvate; | |
| | | | | involved in fatty | propanoate | |
| | | | | acid | metabolism | |
| | | | | biosynthesis | | |
| SEQ ID | 23 | caffeic acid O-methyltransferase | gi\|45444737 | — | Biosynthesis of | 2.1.1.68 Transferases, |
| NO: 6 | | [Vanilla planifolia] | | | secondary | methyltransferase |
| | | | | | metabolites | |
| SEQ ID | 9 | catalase 2 [Elaeis guineensis] | gi\|192910916 | Hydrogen | Energy; methane; | EC1.11.1.6 Catalase |
| NO: 7 | | | | peroxidase | amino acid and | |
| | | | | metabolism | tryptophan | |
| | | | | activity | metabolism | |
| SEQ ID | 59 | conserved hypothetical protein | gi\|223539131 | — | — | — |
| NO: 8 | | [Ricinus communis] | | | | |
| SEQ ID | 73 | fibrillin-like protein [Elaeis | gi\|32250939 | Fibrillin = | — | — |
| NO: 9 | | guineensis] | | microfibrils | | |
| | | | | surrounding | | |
| | | | | amorphous elastin | | |
| SEQ ID | 46 | FQR1 (FLAVODOXIN-LKE | gi\|15239652 | Quinone reductase | — | — |
| NO: 10 | | QUINONE REDUCTASE 1) | | family, involved in | | |
| | | [Arabidopsis thaliana] | | redox and auxin | | |
| | | | | response | | |
| SEQ ID | 74 | fructose-bisphosphate aldolase | gi\|192910908 | — | Carbohydrate | EC4.1.2.13 Lyases, |
| NO: 11 | | [Elaeis guineensis] | | | metabolism, energy | Aldehydelyases |
| | | | | | metabolism | |
| SEQ ID | 26 | glyceraldehyde 3-phosphate | gi\|82400215 | Intermediate in | Carbohydrate | EC1.2.1.9 GAP (NADP); |
| NO: 12 | | dehydrogenase [Elaeis guineensis] | | glycolysis, | metabolism, | EC1.2.1.12 GAP A; |
| | | | | gluconeogenesis | glycolysis and | EC1.2.1.59 GAP2 (NAD(P)); |
| | | | | and photosynthesis | gluconeogenesis, | EC1.2.1.13 GAP2 (NADP + |
| | | | | | energy metabolism, | phosphorylating); EC1.2.1.12 |
| | | | | | carbon fixation in | GAPDHS (spermatogenic) |
| | | | | | photosynthesis | |
| SEQ ID | 19 | H0825G02.11 [Oryza sativa | gi\|116309131 | Contains | — | — |
| NO: 13 | | (indica cultivar-group)] | | transposase | | |
| | | | | domain; | | |
| | | | | Transposase = | | |
| | | | | enzyme binds to | | |
| | | | | end of transposon | | |
| | | | | to catalyze | | |
| | | | | movement of | | |
| | | | | transposon | | |
| SEQ ID | 56 | large subunit of ribulose-1,5- | gi\|74267421 | RuBisCO = used in | Glyoxylate and | EC4.1.1.39 ribulose- |
| NO: 14 | | bisphosphate carboxylase/oxygenase | | Calvin cycle to | dicarboxylate | bisphosphate carboxylase |
| | | [Chlorogonium elongatum] | | catalyze carbon | metabolism; | |
| | | | | fixation | photosynthesis | |
| | | | | | carbon fixation | |
| SEQ ID | 48 | Lea1P [Daucus carota] | gi\|10945667 | A small nuclear | — | — |
| NO: 15 | | | | ribonucleoprotein | | |
| | | | | (snRNP), required | | |
| | | | | in prespliceosome | | |
| | | | | formation | | |
| SEQ ID | 5 | methionine synthase protein | gi\|18483235 | Oxidoreductases | Cysteine and | EC2.1.1.13; methionine |
| NO: 16 | | [Sorghum bicolor] | | family | methionine | synthase |
| | | | | | metabolism | |
| SEQ ID | 51 | mitochondrial peroxiredoxin | gi\|47775654 | Antioxidant | Unclassified | EC1.11.1.15 Peroxiredoxin; |
| NO: 17 | | [Pisum sativum] | | enzyme; regulates | | EC1.8.98.2 Peroxiredoxin- |
| | | | | changes in | | (S-hydroxy-S-oxocystein) |
| | | | | phosphorylation, | | reductase |
| | | | | redox & | | |
| | | | | oligomerization | | |
| SEQ ID | 58 | Os02g0753300 [Oryza sativa | gi\|115448731 | Homolog = A. | — | — |
| NO: 18 | | (japonica cultivar-group)] | | thaliana lipid- | | |
| | | | | associated family | | |
| | | | | protein | | |
| SEQ ID | 66 | Os05g0482700 [Oryza sativa | gi\|115464537 | Unknown protein | — | — |
| NO: 19 | | (japonica cultivar-group)] | | | | |
| SEQ ID | 68 | Os12g0163700 [Oryza sativa | gi\|115487492 | Homologous to | — | — |
| NO: 20 | | (japonica cultivar-group)] | | ACTG2-actin | | |
| | | | | gamma 2 | | |
| SEQ ID | 7 | OSJNBb0085F13.17 [Oryza sativa | gi\|38345312 | Heat shock protein | NOD-like receptor | — |
| NO: 21 | | (japonica cultivar-group)] | | 90 homolog | signaling pathway | |
| SEQ ID | 34 | predicted protein [Ostreococcus | gi\|145345862 | Contains a p23-like | — | — |
| NO: 22 | | lucimarinus CCE9901] | | domain and several | | |
| | | | | HSP90 binding | | |
| | | | | sites | | |
| SEQ ID | 79 | predicted protein [Physcomitrella | gi\|168027637 | Ortholog of | — | — |
| NO: 23 | | patens subsp. patens] | | Glutathione S- | | |
| | | | | transferase theta | | |
| | | | | from A. thaliana | | |
| SEQ ID | 20 | predicted protein [Populus | gi\|224129424 | — | — | — |
| NO: 24 | | trichocarpa] | | | | |
| SEQ ID | 55 | PREDICTED: hypothetical protein | gi\|225434277 | Contains a | — | — |
| NO: 25 | | isoform 1 [Vitis vinifera] | | cystathione beta- | | |
| | | | | synthase (CBS) | | |
| | | | | domain | | |
| SEQ ID | 33 | PREDICTED: similar to nascent | gi\|225470846 | — | — | — |
| NO: 26 | | polypeptide associated complex | | | | |
| | | alpha-like protein 1 | | | | |
| | | [Arabidopsis thaliana] | | | | |
| SEQ ID | 24 | proline iminopeptidase, putative | gi\|223538638 | — | Amino acid | EC3.4.11.5 Hydrolases, |
| NO: 27 | | [Ricinus communis] | | | metabolism | Aminopeptidases |
| SEQ ID | 39 | protein transporter [Elaeis guineensis] | gi\|192910836 | Transmembrane | — | — |
| NO: 28 | | | | protein that helps | | |
| | | | | certain substances | | |
| | | | | to cross | | |
| | | | | the membrane | | |
| SEQ ID | 12 | putative NBS-LRR disease resistance | gi\|38636971 | Nucleotide-binding | — | — |
| NO: 29 | | protein homologue [Oryza sativa | | sequence-leucine | | |
| | | Japonica Group] | | rich region, plant | | |
| | | | | defensive system | | |
| SEQ ID | 32 | Ran GTPase binding protein, putative | gi\|223534074 | Ran = Ras-related | Purine | EC3.1.5.1 dGTPase; |
| NO: 30 | | [Ricinus communis] | | nuclear protein. | metabolism | EC3.6.5.3 protein- |
| | | | | Involved in | | synthesizing GTPase; |
| | | | | transportation of | | EC3.6.5.5 dynamic |
| | | | | cell nucleus during | | GTPase; EC3.6.5.2 small |
| | | | | interphase | | monomeric GTPase; |
| | | | | | | EC3.6.5.4 signal |
| | | | | | | recognition particle |
| | | | | | | GTPase |
| SEQ ID | 37 | Triosephosphate isomerase, | gi\|1174745 | Involved in | Glycolysis/ | EC5.3.1.1 triosephosphate |
| NO: 31 | | chloroplastic [Spinacia oleracea] | | glycolysis and | gluconeogenesis; | isomerase (TIM) |
| | | | | energy production. | fructose and | |
| | | | | | mannose | |
| | | | | | metabolism; | |
| | | | | | inositol phosphate | |
| | | | | | metabolism | |
| SEQ ID | 13 | V-type proton ATPase catalytic subunit | gi\|137460 | Catalyzes ATP to | Energy metabolism; | EC3.6.3.9 ATP1A Na+/K+ |
| NO: 32 | | A [Daucus carota] | | ADP to release | photosynthesis | exchanging ATPase alpha |
| | | | | energy | | subunit; EC3.6.3.14 |
| | | | | | | ATPF F-type H+-transporting |
| | | | | | | ATPase subunit a, b and c |
| SEQ ID | 84 | regulator of ribonuclease activity A | gi\|195638112 | Inhibits the | — | — |
| NO: 33 | | [Zea mays] | | endonuclease | | |
| | | | | activity | | |
| SEQ ID | 36 | retroelement pol polyprotein-like | gi\|9759493 | Transposons via | — | — |
| NO: 34 | | [Arabidopsis thaliana] | | RNA intermediates. | | |
| SEQ ID | 72 | ribosomal protein L10 [Elaeis | gi\|192910684 | Mitochodrial | Ribosome | — |
| NO: 35 | | guineensis] | | protein, key protein | | |
| | | | | for large ribosomal | | |
| | | | | subunit | | |
| | | | | assembly and | | |
| | | | | protein synthesis | | |
| SEQ ID | 75 | short chain type dehydrogenase, | gi\|223550344 | NAD or NADP- | Unclassified | EC1.1.1.100 Short chain |
| NO: 36 | | putative [Ricinus communis] | | dependent | | type dehydrogenase |
| | | | | oxidoreductases | | |
| SEQ ID | 53 | temperature-induced lipocalin [Elaeis | gi\|192911934 | Lipocalin = proteins | — | — |
| NO: 37 | | guineensis] | | that transport lipid | | |
| | | | | & steroids. TIL is | | |
| | | | | responsive to heat | | |
| | | | | stress | | |
| SEQ ID | 38 | unknown [Picea sitchensis] | gi\|224285125 | Contains conserved | — | — |
| NO: 38 | | | | region of 3- | | |
| | | | | oxoacyl-(acyl- | | |
| | | | | carrier-protein) | | |
| | | | | reductase & | | |
| | | | | Rossmann-fold | | |
| | | | | NAD(P)(+)-binding | | |
| | | | | proteins | | |
| SEQ ID | 81 | 17.6 kDa class I small heat shock | gi\|15220832 | Induced by heat | — | — |
| NO: 39 | | protein (HSP17.6C-CI) (AA 1-156) | | stress and involved | | |
| | | [Arabidopsis thaliana] | | in chaperone | | |
| | | | | functions by | | |
| | | | | protecting the | | |
| | | | | proteins from | | |
| | | | | irreversible | | |
| | | | | denaturation | | |
| SEQ ID | 49 | ABC1 family protein [Arabidopsis | gi\|79325958 | ABC1 = novel | — | — |
| NO: 40 | | thaliana] | | chaperonins in | | |
| | | | | electron transfer | | |
| SEQ ID | 54 | glutathione peroxidase [Triticum | gi\|148529480 | — | Metabolism of other | EC1.11.1.9 Oxidoreductases, |
| NO: 41 | | monococcum subsp. aegilopoides] | | | amino acids, | Peroxidases |
| | | | | | lipid metabolism | |
| SEQ ID | 41 | glutathione S-transferase [Matricaria | gi\|17385642 | — | Metabolism of other | EC2.5.1.18 Transferases |
| NO: 42 | | chamomilla] | | | amino acids | |
| SEQ ID | 43 | glutathione-s-transferase theta, gst, | gi\|223528475 | — | — | — |
| NO: 43 | | putative [Ricinus communis] | | | | |
| SEQ ID | 63 | phospholipase D [Oryza sativa | gi\|1020415 | Located in plasma | Glycerophospholipid; | EC3.1.4.4 phospholipase D; |
| NO: 44 | | Japonica Group] | | membrane and | ether lipid; arachidonic | EC3.1.4.11 phospholipase C, |
| | | | | catalyzes | acid; linoleic acid; | delta |
| | | | | hydrolysis of | alphalinolcic acid | |
| | | | | phosphatidylcholine | metabolism | |
| SEQ ID | 30 | VFB2 (VIER F-BOX PROTEINE 2); | gi\|18409012 | Involved in | Ubiquitin mediated | EC6.3.2.19 ubiquitin-protein |
| NO: 45 | | ubiquitin-protein ligase [Arabidopsis | | polyubiquitination | proteolysis; Wnt | ligase |
| | | thaliana] | | (marks protein for | and p53 signaling | |
| | | | | degradation by | pathway | |
| | | | | proteosome) | | |

TABLE 2
Details of differential expression of the forty-five proteins.

| | | | Week 12 | Week 16 | Week 18 | High | High | Low | Low |
|---|---|---|---|---|---|---|---|---|---|
| Pro- | Spot | Top Ranked Protein Name | (High | (High | (High | (Week | (Week | (Week | (Week |
| tein | # | [Species] | vs Low) | vs Low) | vs Low) | 12 vs 16) | 12 vs 18) | 12 vs 16) | 12 vs 18) |
| SEQ ID | 65 | 5-methyltetrahydropteroyl- | Upregulated | — | — | Upregulated | Upregulated | Upregulated | Upregulated |
| NO: 1 | | triglutamate-homocysteine | in high | | | in H12 | in H12 | in L12 | in L12 |
| | | methyltransferase, putative, | | | | | | | |
| | | expressed [Oryza sativa] | | | | | | | |
| SEQ ID | 78 | abscisic stress ripening protein | — | — | — | Upregulated | Upregulated | Upregulated | Upregulated |
| NO: 2 | | [Musa acuminata subsp. | | | | in H12 | in H12 | in L12 | in L12 |
| | | burmannicoides] | | | | | | | |
| SEQ ID | 22 | actin 6 [Populus trichocarpa] | — | Upregulated | — | Upregulated | — | — | — |
| NO: 3 | | | | in low | | in H12 | | | |
| SEQ ID | 76 | actin E [Elaeis guineensis] | — | — | — | Upregulated | Upregulated | Upregulated | Upregulated |
| NO: 4 | | | | | | in H16 | in H18 | in L16 | in L18 |
| SEQ ID | 67 | biotin carboxylase precursor | — | — | — | Upregulated | Upregulated | Upregulated | Upregulated |
| NO: 5 | | [Glycine max] | | | | in H16 | in H18 | in L16 | in L18 |
| SEQ ID | 23 | caffeic acid O-methyltransferase | Upregulated | Upregulated | — | Upregulated | Upregulated | — | Upregulated |
| NO: 6 | | [Vanilla planifolia] | in low | in low | | in H12 | in H12 | | in L12 |
| SEQ ID | 9 | catalase 2 [Elaeis guineensis] | Upregulated | — | Upregulated | Upregulated | Upregulated | Upregulated | Upregulated |
| NO: 7 | | | in high | | in high | in H12 | in H12 | in L12 | in L12 |
| SEQ ID | 59 | conserved hypothetical protein | — | Upregulated | — | Upregulated | Upregulatcd | — | Upregulated |
| NO: 8 | | [Ricinus communis] | | in high | | in H16 | in H18 | | in L18 |
| SEQ ID | 73 | fibrillin-like protein [Elaeis | Upregulated | — | — | Upregulated | — | — | Upregulated |
| NO: 9 | | guineensis] | in high | | | in H12 | | | in L18 |
| SEQ ID | 46 | FQR1 (FLAVODOXIN-LIKE | Upregulated | — | Upregulated | Upregulated | — | — | — |
| NO: 10 | | QUINONE REDUCTASE 1) | in low | | in low | in H16 | | | |
| | | [Arabidopsis thaliana] | | | | | | | |
| SEQ ID | 74 | fructose-bisphosphate aldolase | — | Upregulated | — | Upregulated | Upregulated | Upregulated | Upregulated |
| NO: 11 | | [Elaeis guineensis] | | in high | | in H16 | in H18 | in L16 | in L18 |
| SEQ ID | 26 | glyceraldehyde 3-phosphate | — | Upregulated | — | — | — | — | — |
| NO: 12 | | dehydrogenase [Elaeis | | in low | | | | | |
| | | guineensis] | | | | | | | |
| SEQ ID | 19 | H0825G02.11 [Oryza sativa | — | Upregulated | — | — | Upregulated | Upregulated | — |
| NO: 13 | | (indica cultivar-group)] | | in low | | | in H18 | in L16 | |
| SEQ ID | 56 | large subunit of ribulose-1,5- | — | — | Upregulated | Upregulated | Upregulated | Upregulated | Upregulated |
| NO: 14 | | bisphosphate carboxylase/ | | | in high | in H16 | in H18 | in L16 | in L18 |
| | | oxygenase [Chlorogonium | | | | | | | |
| | | elongatum] | | | | | | | |
| SEQ ID | 48 | Lea1P [Daucus carota] | — | Upregulated | — | Upregulated | — | — | — |
| NO: 15 | | | | in high | | in H16 | | | |
| SEQ ID | 5 | methionine synthase protein | — | Upregulated | — | Upregulated | — | — | — |
| NO: 16 | | [Sorghum bicolor] | | in low | | in H12 | | | |
| SEQ ID | 51 | mitochondrial peroxiredoxin | Upregulated | Upregulated | — | Upregulated | Upregulated | Upregulated | Upregulated |
| NO: 17 | | [Pisum sativum] | in high | in high | | in H16 | in H18 | in L16 | in L18 |
| SEQ ID | 58 | Os02g0753300 [Oryza sativa | — | Upregulated | — | — | — | — | — |
| NO: 18 | | (japonica cultivar-group)] | | in high | | | | | |
| SEQ ID | 66 | Os05g0482700 [Oryza sativa | — | Upregulated | — | Upregulated | Upregulated | — | — |
| NO: 19 | | (japonica cultivar-group)] | | in low | | in H12 | in H12 | | |
| SEQ ID | 68 | Os12g0163700 [Oryza sativa | — | — | — | Upregulated | Upregulated | Upregulated | Upregulated |
| NO: 20 | | (japonica cultivar-group)] | | | | in H16 | in H18 | in L16 | in L18 |
| SEQ ID | 7 | OSJNBb0085F13.17 [Oryza | — | Upregulated | — | Upregulated | — | — | Upregulated |
| NO: 21 | | sativa (japonica cultivar-group)] | | in low | | in H12 | | | in L18 |
| SEQ ID | 34 | predicted protein [Ostreococcus | — | Upregulated | — | Upregulated | — | — | — |
| NO: 22 | | lucimarinus CCE9901] | | in high | | in H16 | | | |
| SEQ ID | 79 | predicted protein | Upregulated | — | — | Upregulated | Upregulated | — | — |
| NO: 23 | | [Physcomitrella patens subsp. | in low | | | in H16 | in H18 | | |
| | | patens] | | | | | | | |
| SEQ ID | 20 | predicted protein [Populus | Upregulated | Upregulated | — | — | Upregulated | — | — |
| NO: 24 | | trichocarpa] | in low | in low | | | in H18 | | |
| SEQ ID | 55 | PREDICTED: hypothetical | Upregulated | — | — | Upregulated | Upregulated | — | Upregulated |
| NO: 25 | | protein isoform 1 [Vitis vinifera] | in high | | | in H12 | in H12 | | in L12 |
| SEQ ID | 33 | PREDICTED: similar to | Upregulated | — | Upregulated | Upregulated | Upregulated | — | Upregulated |
| NO: 26 | | nascent polypeptide associated | in high | | in high | in H12 | in H12 | | in L12 |
| | | complex alpha-like protein 1 | | | | | | | |
| | | [Arabidopsis thaliana] | | | | | | | |
| SEQ ID | 24 | proline iminopeptidase, putative | — | Upregulated | Upregulated | Upregulated | Upregulated | Upregulated | Upregulated |
| NO: 27 | | [Ricinus communis] | | in high | in high | in H16 | in H18 | in L16 | in L18 |
| SEQ ID | 39 | protein transporter [Elaeis | Upregulated | — | — | Upregulated | Upregulated | — | — |
| NO: 28 | | guineensis] | in low | | | in H16 | in H18 | | |
| SEQ ID | 12 | putative NBS-LRR disease | — | Upregulated | — | Upregulated | — | — | — |
| NO: 29 | | resistance protein homologue | | in low | | in H12 | | | |
| | | [Oryza sativa Japonica Group] | | | | | | | |
| SEQ ID | 32 | Ran GTPase binding protein, | Upregulated | — | — | Upregulated | Upregulated | Upregulated | Upregulated |
| NO: 30 | | putative [Ricinus communis] | in high | | | in H16 | in H18 | in L16 | in L18 |
| SEQ ID | 37 | Triosephosphate isomerase, | — | Upregulated | — | — | — | — | — |
| NO: 31 | | chloroplastic [Spinacia oleracea] | | in low | | | | | |
| SEQ ID | 13 | V-type proton ATPase catalytic | — | Upregulated | — | Upregulated | — | — | — |
| NO: 32 | | subunit A [Daucus carota] | | in low | | in H12 | | | |
| SEQ ID | 84 | regulator of ribonuclease | — | — | — | Upregulated | Upregulated | — | Upregulated |
| NO: 33 | | activity A [Zea mays] | | | | in H16 | in H18 | | in L18 |
| SEQ ID | 36 | retroelement pol polyprotein-like | Upregulated | — | — | Upregulated | — | — | — |
| NO: 34 | | [Arabidopsis thaliana] | in low | | | in H16 | | | |
| SEQ ID | 72 | ribosomal protein L10 [Elaeis | Upregulated | — | — | Upregulated | Upregulated | Upregulated | Upregulated |
| NO: 35 | | guineensis] | in low | | | in H16 | in H18 | in L16 | in L18 |
| SEQ ID | 75 | short chain type dehydrogenase, | — | — | — | Upregulated | Upregulated | Upregulated | Upregulated |
| NO: 36 | | putative [Ricinus communis] | | | | in H16 | in H18 | in L16 | in L18 |
| SEQ ID | 53 | temperature-induced lipocalin | — | Upregulated | — | Upregulated | — | — | — |
| NO: 37 | | [Elaeis guineensis] | | in high | | in H16 | | | |
| SEQ ID | 38 | unknown [Picea sitchensis] | — | Upregulated | — | Upregulated | Upregulated | — | Upregulated |
| NO: 38 | | | | in high | | in H16 | in H18 | | in L18 |
| SEQ ID | 81 | 17.6 kDa class I small heat | — | — | — | Upregulated | Upregulated | Upregulated | Upregulated |
| NO: 39 | | shock protein (HSP17.6C-CI) | | | | in H16 | in H18 | in L16 | in L18 |
| | | (AA 1-156) [Arabidopsis | | | | | | | |
| | | thaliana] | | | | | | | |
| SEQ ID | 49 | ABC1 family protein | — | Upregulated | Upregulated | Upregulated | Upregulated | — | — |
| NO: 40 | | [Arabidopsis thaliana] | | in high | in low | in H16 | in H12 | | |
| SEQ ID | 54 | glutathione peroxidase [Triticum | — | Upregulated | — | — | — | — | — |
| NO: 41 | | monococcum subsp. | | in high | | | | | |
| | | aegilopoides] | | | | | | | |
| SEQ ID | 41 | glutathione S-transferase | Upregulated | Upregulated | Upregulated | Upregulated | Upregulated | Upregulated | — |
| NO: 42 | | [Matricaria chamomilla] | in low | in low | in low | in H16 | in H18 | in L16 | |
| SEQ ID | 43 | glutathione-s-transferase theta, | Upregulated | — | Upregulated | Upregulated | Upregulated | — | — |
| NO: 43 | | gst, putative [Ricinus communis] | in low | | in high | in H16 | in H18 | | |
| SEQ ID | 63 | phospholipase D [Oryza sativa | — | Upregulated | — | Upregulated | — | — | Upregulated |
| NO: 44 | | Japonica Group] | | in low | | in H12 | | | in L12 |
| SEQ ID | 30 | VFB2 (VIER F-BOX | — | — | Upregulated | — | — | — | Upregulated |
| NO: 45 | | PROTEINE 2); ubiquitin-protein | | | in high | | | | in L12 |
| | | ligase [Arabidopsis thaliana] | | | | | | | |

TABLE 3
Antibodies against 27 of the 45 unique differentially expressed proteins.

| Antibody | Protein | | | |
|---|---|---|---|---|
| No. | Spot No. | Antibody Ref. No. | Antibody Name | Company |
| 1 | 5 | AB9209 | MTR antibody | Abcam |
| 2 | 7 | AB19104 | HSP90 antibody | Abcam |
| 3 | 9 & 14 | AS09501 | Catalase | Agrisera |
| 4 | 13 | AS09503 | V-ATPase B, vacuolar H+ ATPase subunit B | Agrisera |
| 5 | 22 & 76 | A0480-200 μl | Anti-actin (plant), Clone 10-B3 | Sigma Aldrich |
| 6 | 23 & 25 | AB51984 | COMT | Abcam |
| 7 | 24 | GW22303B-50 μg | Chicken Anti-XPNPEP (X-prolyl aminopeptidase | GenWay |
| | | | (aminopeptidase P)1)(N-193) | |
| 8 | 26 | 10494-1-AP | GAPDH | |
| 9 | 30 | AB90146 | HECT E3 ubiquitin ligase antibody | Abcam |
| 10 | 32 | AS07217 | FtsZ, prokaryotic cell division GTPase | Agrisera |
| 11 | 33 | ABIN337196 | Nascent-polypeptide-associated + Complex + Alpha + | Antibodies-online |
| | | | Polypeptide + (NACA) (Human) | |
| 12 | 37 | AB58329 | Triosephosphate isomerase antibody | Abcam |
| 13 | 41 & 42 | AS09479 | GST class-phi, glutathione S transferase | Agrisera |
| 14 | 43 & 79 | AB55188 | glutathione s-transferase theta 1 | Abcam |
| 15 | 48 | SC-102147 | U2 snRNP A (F-22) | Santa-Cruz |
| 16 | 49 | AB18180 | ABCA1 antibody [AB.H10] | Abcam |
| 17 | 51 | AS05093 | PrxQ, peroxiredoxin, thioredoxin reductase | Agrisera |
| 18 | 53 & 82 | AB61866 | Prostaglandin D Synthase (Lipocalin) antibody | Abcam |
| 19 | 54 | AS04055 | GPX, chloroplastic glutathione peroxidase | Agrisera |
| 20 | 56 | AS03037 | RbcL, Rubisco large subunit, form I and form II | Agrisera |
| 21 | 63 | AS09556 | PLD, phospholipase D | Agrisera |
| 22 | 65 | M4697-50 | Anti-MTR (5-Methyltetrahydrofolate-homocysteine Methyltransferase) | US Biological |
| 23 | 67 | GTX110062 | MCCC1 | GeneTex Inc |
| 24 | 68 | ABIN229498 | 0 actin, Gamma 2, Smooth muscle enteric ACTG2 | Antibodies-online |
| 25 | 72 | 17013-1-AP | RPL10 | Proteintech Group |
| 26 | 74 | AS08294 | ALD, fructose-1,6 bisphosphate aldolase | Agrisera |
| 27 | 80, 81 & 83 | AS07254 | HSP17.6 cytosolic class I HSP 17.6 | Agrisera |

TABLE 4
Dot-blot immunoassay results, as supported by Mann Whitney test.

| Antibody | Antibody Name | Protein | | | Differential |
|---|---|---|---|---|---|
| No. | (Ref. No.) | Spot No. | Top Ranked Protein Name (Species) | Accession No. | Expression |
| 1 | COMT | 23 & 25 | Caffeic acid O-methyltransferase (Vanilla planifolia) | gi/45444737 | Week 12 |
| | (AB51984) | | | | |
| 2 | TIM (AB58329) | 37 | Triosephosphate isomerase, chloroplastic (Spinacia oleracea) | gi/1174745 | Weeks 12 & 16 |
| 3 | GST theta | 43 | Glutathione-s-transferase theta, gst, putative (Ricinus communis) | gi/223528475 | Week 20 |
| | (AB55188) | | | | |
| 4 | GST theta | 79 | Predicted protein (Physcomitrella patens subsp. Patens) | gi/168027637 | Week 20 |
| | (AB55188) | | | | |
| 5 | ABCA1 | 49 | ABC1 family protein (Arabidopsis thaliana) | gi/79325958 | Week 12 |
| | (AB18180) | | | | |
| 6 | NACA | 33 | Predicted: Similar to nascent polypeptide associated complex | gi/225470846 | Weeks 12 & 14 |
| | (ABIN337196) | | alpha-like protein 1 | | |
| 7 | GPX (AS04055) | 54 | Glutathione peroxidase (Triticum monococcum subsp. aegilopoides) | gi/148529480 | Week 12 |
| 8 | RbcL(AS03037) | 56 | Large subunit of ribulose-1,5-bisphosphate carboxylase/oxygenase | gi/74267421 | Week 22 |
| 9 | RPL10 (17013- | 72 | Ribosomal protein L10 (Elaeis guineensis) | gi/192910684 | Week 14 |
| | 1-AP) | | | | |
| 10 | ALD (AS08294) | 74 | Fructose-bisphosphate aldolase (Elaeis guineensis) | gi/192910908 | Week 12 |
| 11 | HSP17.6 | 80, 81 & | 17.6 kDa class I small heat shock protein (HSP17.6C-CI) | gi/15220832 | Week 22 |
| | (AS07254) | 83 | (Arabidopsis thaliana) | | |

1. A method for obtaining a high-yielding oil palm plant comprising:
(i) determining the level of a protein in mesocarp tissue of a fruit of a parental oil palm plant, wherein the protein is selected from the group consisting of 5-methyltetrahydropteroyltriglutamate-homocysteine methyltransferase, abscisic stress ripening protein, actin 6, actin E, biotin carboxylase precursor, caffeic acid O-methyltransferase, catalase 2, conserved-hypothetical-protein-of-Ricinus-communis ortholog, fibrillin-like protein, flavodoxin-like quinone reductase 1, fructose-bisphosphate aldolase, glyceraldehyde 3-phosphate dehydrogenase, H0825G02.11 ortholog, large subunit of ribulose-1,5-bisphosphate carboxylase/oxygenase, LealP, methionine synthase protein, mitochondrial peroxiredoxin, Os02g0753300 ortholog, Os05g0482700 ortholog, Os12g0163700 ortholog, OSJNBb0085F13.17 ortholog, predicted-protein-of-Ostreococcus-lucimarinus-CCE9901 ortholog, predicted-protein-of-Physcomitrella patens-subsp.-patens ortholog, predicted-protein-of-Populus-trichocarpa ortholog, hypothetical-protein-isoform-1-of-Vitis-vinifera ortholog, nascent polypeptide associated complex alpha, proline iminopeptidase, protein transporter, putative-NBS-LRR-disease-resistance-protein-homologue-of-Oryza-sativa-Japonica-Group ortholog, Ran GTPase binding protein, chloroplastic triosephosphate isomerase, V-type proton ATPase catalytic subunit A, regulator of ribonuclease activity A, retroelement pol polyprotein-like ortholog, ribosomal protein L10, short chain type dehydrogenase, temperature-induced lipocalin, and unknown-protein-of-Picea-sitchensis ortholog;
(ii) determining whether there is a difference between the level of the protein in the mesocarp tissue of the fruit of the parental oil palm plant and the level of the protein in mesocarp tissue of a fruit of a reference oil palm plant; and
(iii) selecting progeny of the parental oil palm plant based on the difference to obtain the high-yielding oil palm plant.

2. The method of claim 1, wherein the 5-methyltetrahydropteroyltriglutamate-homocysteine methyltransferase comprises SEQ ID NO: 1, the abscisic stress ripening protein comprises SEQ ID NO: 2, the actin 6 comprises SEQ ID NO: 3, the actin E comprises SEQ ID NO: 4, the biotin carboxylase precursor comprises SEQ ID NO: 5, the caffeic acid O-methyltransferase comprises SEQ ID NO: 6, the catalase 2 comprises SEQ ID NO: 7, the conserved-hypothetical-protein-of-Ricinus-communis ortholog comprises SEQ ID NO: 8, the fibrillin-like protein comprises SEQ ID NO: 9, the flavodoxin-like quinone reductase 1 comprises SEQ ID NO: 10, the fructose-bisphosphate aldolase comprises SEQ ID NO: 11, the glyceraldehyde 3-phosphate dehydrogenase comprises SEQ ID NO: 12, the H0825G02.11 ortholog comprises SEQ ID NO: 13, the large subunit of ribulose-1,5-bisphosphate carboxylase/oxygenase comprises SEQ ID NO: 14, the LealP comprises SEQ ID NO: 15, the methionine synthase protein comprises SEQ ID NO: 16, the mitochondrial peroxiredoxin comprises SEQ ID NO: 17, the Os02g0753300 ortholog comprises SEQ ID NO: 18, the Os05g0482700 ortholog comprises SEQ ID NO: 19, the Os12g0163700 ortholog comprises SEQ ID NO: 20, the OSJNBb0085F13.17 ortholog comprises SEQ ID NO: 21, the predicted-protein-of-Ostreococcus-lucimarinus-CCE9901 ortholog comprises SEQ ID NO: 22, the predicted-protein-of-Physcomitrella patens-subsp.-patens ortholog comprises SEQ ID NO: 23, the predicted-protein-of-Populus-trichocarpa ortholog comprises SEQ ID NO: 24, the hypothetical-protein-isoform-1-of-Vitis-vinifera ortholog comprises SEQ ID NO: 25, the nascent polypeptide associated complex alpha comprises SEQ ID NO: 26, the proline iminopeptidase comprises SEQ ID NO: 27, the protein transporter comprises SEQ ID NO: 28, the putative-NBS-LRR-disease-resistance-protein-homologue-of-Oryza-sativa-Japonica-Group ortholog comprises SEQ ID NO: 29, the Ran GTPase binding protein comprises SEQ ID NO: 30, the chloroplastic triosephosphate isomerase comprises SEQ ID NO: 31, the V-type proton ATPase catalytic subunit A comprises SEQ ID NO: 32, the regulator of ribonuclease activity A comprises SEQ ID NO: 33, the retroelement pol polyprotein-like ortholog comprises SEQ ID NO: 34, the ribosomal protein L10 comprises SEQ ID NO: 35, the short chain type dehydrogenase comprises SEQ ID NO: 36, the temperature-induced lipocalin comprises SEQ ID NO: 37, and the unknown-protein-of-Picea-sitchensis ortholog comprises SEQ ID NO: 38.

3. The method of claim 1 or 2, wherein the difference is that the level of the 5-methyltetrahydropteroyltriglutamate-homocysteine methyltransferase in the mesocarp tissue of the fruit of the parental oil palm plant 11 to 13 weeks after pollination thereof is higher than the level of the 5-methyltetrahydropteroyltriglutamate-homocysteine methyltransferase in the mesocarp tissue of the fruit of the reference oil palm plant 11 to 13 weeks after pollination thereof.

4. The method of claim 1 or 2, wherein the difference is that the level of the abscisic stress ripening protein in the mesocarp tissue of the fruit of the parental oil palm plant 11 to 13 weeks after pollination thereof is higher than the level of the abscisic stress ripening protein in the mesocarp tissue of the fruit of the reference oil palm plant 15 to 19 weeks after pollination thereof.

5. The method of claim 1 or 2, wherein the difference is that the level of the actin 6 in the mesocarp tissue of the fruit of the parental oil palm plant 15 to 17 weeks after pollination thereof is lower than the level of the actin 6 in the mesocarp tissue of the fruit of the reference oil palm plant 15 to 17 weeks after pollination thereof.

6. The method of claim 1 or 2, wherein the difference is that the level of the actin E in the mesocarp tissue of the fruit of the parental oil palm plant 15 to 19 weeks after pollination thereof is higher than the level of the actin E in the mesocarp tissue of the fruit of the reference oil palm plant 11 to 13 weeks after pollination thereof.

7. The method of claim 1 or 2, wherein the difference is that the level of the biotin carboxylase precursor in the mesocarp tissue of the fruit of the parental oil palm plant 15 to 19 weeks after pollination thereof is higher than the level of the biotin carboxylase precursor in the mesocarp tissue of the fruit of the reference oil palm plant 11 to 13 weeks after pollination thereof.

8. The method of claim 1 or 2, wherein the difference is that the level of caffeic acid O-methyltransferase in the mesocarp tissue of the fruit of the parental oil palm plant 11 to 17 weeks after pollination thereof is lower than the level of caffeic acid O-methyltransferase in the mesocarp tissue of the fruit of the reference oil palm plant 11 to 17 weeks after pollination thereof.

9. The method of claim 1 or 2, wherein the difference is that the level of the catalase 2 in the mesocarp tissue of the fruit of the parental oil palm plant 11 to 19 weeks after pollination thereof is higher than the level of the catalase 2 in the mesocarp tissue of the fruit of the reference oil palm plant 11 to 19 weeks after pollination thereof.

10. The method of claim 1 or 2, wherein the difference is that the level of the conserved-hypothetical-protein-of-Ricinus-communis ortholog in the mesocarp tissue of the fruit of the parental oil palm plant 15 to 17 weeks after pollination thereof is higher than the level of the conserved-hypothetical-protein-of-Ricinus-communis ortholog in the mesocarp tissue of the fruit of the reference oil palm plant 15 to 17 weeks after pollination thereof.

11. The method of claim 1 or 2, wherein the difference is that the level of the fibrillin-like protein in the mesocarp tissue of the fruit of the parental oil palm plant 11 to 13 weeks after pollination thereof is higher than the level of the fibrillin-like protein in the mesocarp tissue of the fruit of the reference oil palm plant 11 to 13 weeks after pollination thereof.

12. The method of claim 1 or 2, wherein the difference is that the level of the flavodoxin-like quinone reductase 1 in the mesocarp tissue of the fruit of the parental oil palm plant 11 to 19 weeks after pollination thereof is lower than the level of the flavodoxin-like quinone reductase 1 in the mesocarp tissue of the fruit of the reference oil palm plant 11 to 19 weeks after pollination thereof.

13. The method of claim 1 or 2, wherein the difference is that the level of the fructose-bisphosphate aldolase in the mesocarp tissue of the fruit of the parental oil palm plant 15 to 17 weeks after pollination thereof is higher than the level of the fructose-bisphosphate aldolase in the mesocarp tissue of the fruit of the reference oil palm plant 15 to 17 weeks after pollination thereof.

14. The method of claim 1 or 2, wherein the difference is that the level of the glyceraldehyde 3-phosphate dehydrogenase in the mesocarp tissue of the fruit of the parental oil palm plant 15 to 17 weeks after pollination thereof is lower than the level of the glyceraldehyde 3-phosphate dehydrogenase in the mesocarp tissue of the fruit of the reference oil palm plant 15 to 17 weeks after pollination thereof.

15. The method of claim 1 or 2, wherein the difference is that the level of the H0825G02.11 ortholog in the mesocarp tissue of the fruit of the parental oil palm plant 15 to 17 weeks after pollination thereof is lower than the level of the H0825G02.11 ortholog in the mesocarp tissue of the fruit of the reference oil palm plant 15 to 17 weeks after pollination thereof.

16. The method of claim 1 or 2, wherein the difference is that the level of the large subunit of ribulose-1,5-bisphosphate carboxylase/oxygenase in the mesocarp tissue of the fruit of the parental oil palm plant 17 to 19 weeks after pollination thereof is higher than the level of the large subunit of ribulose-1,5-bisphosphate carboxylase/oxygenase in the mesocarp tissue of the fruit of the reference oil palm plant 17 to 19 weeks after pollination thereof.

17. The method of claim 1 or 2, wherein the difference is that the level of the LealP in the mesocarp tissue of the fruit of the parental oil palm plant 15 to 17 weeks after pollination thereof is higher than the level of the LealP in the mesocarp tissue of the fruit of the reference oil palm plant 15 to 17 weeks after pollination thereof.

18. The method of claim 1 or 2, wherein the difference is that the level of the methionine synthase protein in the mesocarp tissue of the fruit of the parental oil palm plant 15 to 17 weeks after pollination thereof is lower than the level of the methionine synthase protein in the mesocarp tissue of the fruit of the reference oil palm plant 15 to 17 weeks after pollination thereof.

19. The method of claim 1 or 2, wherein the difference is that the level of the mitochondrial peroxiredoxin in the mesocarp tissue of the fruit of the parental oil palm plant 11 to 17 weeks after pollination thereof is higher than the level of the mitochondrial peroxiredoxin in the mesocarp tissue of the fruit of the reference oil palm plant 11 to 17 weeks after pollination thereof.

20. The method of claim 1 or 2, wherein the difference is that the level of the Os02g0753300 ortholog in the mesocarp tissue of the fruit of the parental oil palm plant 15 to 17 weeks after pollination thereof is higher than the level of the Os02g0753300 ortholog in the mesocarp tissue of the fruit of the reference oil palm plant 15 to 17 weeks after pollination thereof.

21. The method of claim 1 or 2, wherein the difference is that the level of the Os05g0482700 ortholog in the mesocarp tissue of the fruit of the parental oil palm plant 15 to 17 weeks after pollination thereof is lower than the level of the Os05g0482700 ortholog in the mesocarp tissue of the fruit of the reference oil palm plant 15 to 17 weeks after pollination thereof.

22. The method of claim 1 or 2, wherein the difference is that the level of the Os12g0163700 ortholog in the mesocarp tissue of the fruit of the parental oil palm plant 15 to 19 weeks after pollination thereof is higher than the level of the Os12g0163700 ortholog in the mesocarp tissue of the fruit of the reference oil palm plant 11 to 13 weeks after pollination thereof.

23. The method of claim 1 or 2, wherein the difference is that the level of the OSJNBb0085F13.17 ortholog in the mesocarp tissue of the fruit of the parental oil palm plant 15 to 17 weeks after pollination thereof is lower than the level of the OSJNBb0085F13.17 ortholog in the mesocarp tissue of the fruit of the reference oil palm plant 15 to 17 weeks after pollination thereof.

24. The method of claim 1 or 2, wherein the difference is that the level of the predicted-protein-of-Ostreococcus-lucimarinus-CCE9901 ortholog in the mesocarp tissue of the fruit of the parental oil palm plant 15 to 17 weeks after pollination thereof is higher than the level of the predicted-protein-of-Ostreococcus-lucimarinus-CCE9901 ortholog in the mesocarp tissue of the fruit of the reference oil palm plant 15 to 17 weeks after pollination thereof.

25. The method of claim 1 or 2, wherein the difference is that the level of the predicted-protein-of-Physcomitrella patens-subsp.-patens ortholog in the mesocarp tissue of the fruit of the parental oil palm plant 11 to 13 weeks after pollination thereof is lower than the level of the predicted-protein-of-Physcomitrella patens-subsp.-patens ortholog in the mesocarp tissue of the fruit of the reference oil palm plant 11 to 13 weeks after pollination thereof.

26. The method of claim 1 or 2, wherein the difference is that the level of the predicted-protein-of-Populus-trichocarpa ortholog in the mesocarp tissue of the fruit of the parental oil palm plant 11 to 17 weeks after pollination thereof is lower than the level of the predicted-protein-of-Populus-trichocarpa ortholog in the mesocarp tissue of the fruit of the reference oil palm plant 11 to 17 weeks after pollination thereof.

27. The method of claim 1 or 2, wherein the difference is that the level of the hypothetical-protein-isoform-1-of-Vitis-vinifera ortholog in the mesocarp tissue of the fruit of the parental oil palm plant 11 to 13 weeks after pollination thereof is higher than the level of the hypothetical-protein-isoform-1-of-Vitis-vinifera ortholog in the mesocarp tissue of the fruit of the reference oil palm plant 11 to 13 weeks after pollination thereof.

28. The method of claim 1 or 2, wherein the difference is that the level of the nascent polypeptide associated complex alpha in the mesocarp tissue of the fruit of the parental oil palm plant 11 to 19 weeks after pollination thereof is higher than the level of the nascent polypeptide associated complex alpha in the mesocarp tissue of the fruit of the reference oil palm plant 11 to 19 weeks after pollination thereof.

29. The method of claim 1 or 2, wherein the difference is that the level of the proline iminopeptidase in the mesocarp tissue of the fruit of the parental oil palm plant 15 to 19 weeks after pollination thereof is higher than the level of the proline iminopeptidase in the mesocarp tissue of the fruit of the reference oil palm plant 15 to 19 weeks after pollination thereof.

30. The method of claim 1 or 2, wherein the difference is that the level of the protein transporter in the mesocarp tissue of the fruit of the parental oil palm plant 11 to 13 weeks after pollination thereof is lower than the level of the protein transporter in the mesocarp tissue of the fruit of the reference oil palm plant 11 to 13 weeks after pollination thereof.

31. The method of claim 1 or 2, wherein the difference is that the level of the putative-NBS-LRR-disease-resistance-protein-homologue-of-Oryza-sativa-Japonica-Group ortholog in the mesocarp tissue of the fruit of the parental oil palm plant 15 to 17 weeks after pollination thereof is lower than the level of the putative-NBS-LRR-disease-resistance-protein-homologue-of-Oryza-sativa-Japonica-Group ortholog in the mesocarp tissue of the fruit of the reference oil palm plant 15 to 17 weeks after pollination thereof.

32. The method of claim 1 or 2, wherein the difference is that the level of the Ran GTPase binding protein in the mesocarp tissue of the fruit of the parental oil palm plant 11 to 13 weeks after pollination thereof is higher than the level of the Ran GTPase binding protein in the mesocarp tissue of the fruit of the reference oil palm plant 11 to 13 weeks after pollination thereof.

33. The method of claim 1 or 2, wherein the difference is that the level of the chloroplastic triosephosphate isomerase in the mesocarp tissue of the fruit of the parental oil palm plant 15 to 17 weeks after pollination thereof is lower than the level of the chloroplastic triosephosphate isomerase in the mesocarp tissue of the fruit of the reference oil palm plant 15 to 17 weeks after pollination thereof.

34. The method of claim 1 or 2, wherein the difference is that the level of the V-type proton ATPase catalytic subunit A in the mesocarp tissue of the fruit of the parental oil palm plant 15 to 17 weeks after pollination thereof is lower than the level of the V-type proton ATPase catalytic subunit A in the mesocarp tissue of the fruit of the reference oil palm plant 15 to 17 weeks after pollination thereof.

35. The method of claim 1 or 2, wherein the difference is that the level of the regulator of ribonuclease activity A in the mesocarp tissue of the fruit of the parental oil palm plant 15 to 19 weeks after pollination thereof is higher than the level of the regulator of ribonuclease activity A in the mesocarp tissue of the fruit of the reference oil palm plant 11 to 13 weeks after pollination thereof.

36. The method of claim 1 or 2, wherein the difference is that the level of the retroelement pol polyprotein-like ortholog in the mesocarp tissue of the fruit of the parental oil palm plant 11 to 13 weeks after pollination thereof is lower than the level of the retroelement pol polyprotein-like ortholog in the mesocarp tissue of the fruit of the reference oil palm plant 11 to 13 weeks after pollination thereof.

37. The method of claim 1 or 2, wherein the difference is that the level of the ribosomal protein L10 in the mesocarp tissue of the fruit of the parental oil palm plant 11 to 13 weeks after pollination thereof is lower than the level of the ribosomal protein L10 in the mesocarp tissue of the fruit of the reference oil palm plant 11 to 13 weeks after pollination thereof.

38. The method of claim 1 or 2, wherein the difference is that the level of the short chain type dehydrogenase in the mesocarp tissue of the fruit of the parental oil palm plant 15 to 19 weeks after pollination thereof is higher than the level of the short chain type dehydrogenase in the mesocarp tissue of the fruit of the reference oil palm plant 11 to 13 weeks after pollination thereof.

39. The method of claim 1 or 2, wherein the difference is that the level of the temperature-induced lipocalin in the mesocarp tissue of the fruit of the parental oil palm plant 15 to 17 weeks after pollination thereof is higher than the level of the temperature-induced lipocalin in the mesocarp tissue of the fruit of the reference oil palm plant 15 to 17 weeks after pollination thereof.

40. The method of claim 1 or 2, wherein the difference is that the level of the unknown-protein-of-Picea-sitchensis ortholog in the mesocarp tissue of the fruit of the parental oil palm plant 15 to 17 weeks after pollination thereof is higher than the level of the unknown-protein-of-Picea-sitchensis ortholog in the mesocarp tissue of the fruit of the reference oil palm plant 15 to 17 weeks after pollination thereof.

41. The method of claim 1 or 2, further comprising:
(i) determining the level of at least another of the proteins in the mesocarp tissue of the fruit of the parental oil palm plant;
(ii) determining whether there is a difference between the level of the other protein in the mesocarp tissue of the fruit of the parental oil palm plant and the level of the other protein in the mesocarp tissue of the fruit of the reference oil palm plant; and
(iii) selecting the progeny of the parental oil palm plant based also on the difference with respect to the levels of the other protein to obtain the high-yielding oil palm plant.

42. The method of claim 1 or 2, wherein the difference is determined by a technique selected from the group consisting of two-dimensional fluorescence difference gel electrophoresis, antibody-based detection, immunoblot detection, and dot-blot detection.

43. The method of any one of claims 1 to 42, wherein:
the parental oil palm plant is a dura breeding stock plant;
the progeny comprises an oil palm plant selected from the group consisting of a dura breeding stock plant and a tenera agricultural production plant; and
the high-yielding oil palm plant is selected from the group consisting of a dura breeding stock plant and a tenera agricultural production plant.

44. The method of any one of claims 1 to 42, wherein:
the parental oil palm plant is a tenera breeding stock plant;
the progeny comprises an oil palm plant selected from the group consisting of a tenera breeding stock plant, a pisifera breeding stock plant, and a tenera agricultural production plant; and
the high-yielding oil palm plant is selected from the group consisting of a tenera breeding stock plant and a tenera agricultural production plant.

45. A method for obtaining palm oil from a high-yielding oil palm plant comprising:
(i) obtaining the high-yielding oil palm plant by the method of any one of claims 1 to 44; and
(ii) isolating palm oil from a fruit of the high-yielding oil palm plant.

46. A method for predicting oil yield of a test oil palm plant comprising:
(i) determining the level of a protein in mesocarp tissue of a fruit of the test oil palm plant, wherein the protein is selected from the group consisting of 5-methyltetrahydropteroyltriglutamate-homocysteine methyltransferase, abscisic stress ripening protein, actin 6, actin E, biotin carboxylase precursor, caffeic acid O-methyltransferase, catalase 2, conserved-hypothetical-protein-of-Ricinus-communis ortholog, fibrillin-like protein, flavodoxin-like quinone reductase 1, fructose-bisphosphate aldolase, glyceraldehyde 3-phosphate dehydrogenase, H0825G02.11 ortholog, large subunit of ribulose-1,5-bisphosphate carboxylase/oxygenase, LealP, methionine synthase protein, mitochondrial peroxiredoxin, Os02g0753300 ortholog, Os05g0482700 ortholog, Os12g0163700 ortholog, OSJNBb0085F13.17 ortholog, predicted-protein-of-Ostreococcus-lucimarinus-CCE9901 ortholog, predicted-protein-of-Physcomitrella patens-subsp.-patens ortholog, predicted-protein-of-Populus-trichocarpa ortholog, hypothetical-protein-isoform-1-of-Vitis-vinifera ortholog, nascent polypeptide associated complex alpha, proline iminopeptidase, protein transporter, putative-NBS-LRR-disease-resistance-protein-homologue-of-Oryza-sativa-Japonica-Group ortholog, Ran GTPase binding protein, chloroplastic triosephosphate isomerase, V-type proton ATPase catalytic subunit A, regulator of ribonuclease activity A, retroelement pol polyprotein-like ortholog, ribosomal protein L10, short chain type dehydrogenase, temperature-induced lipocalin, and unknown-protein-of-Picea-sitchensis ortholog;
(ii) determining whether there is a difference between the level of the protein in the mesocarp tissue of the fruit of the test oil palm plant and the level of the protein in mesocarp tissue of a fruit of a reference oil palm plant; and
(iii) predicting the oil yield of the test oil palm plant based on the difference.

47. The method of claim 46, wherein the 5-methyltetrahydropteroyltriglutamate-homocysteine methyltransferase comprises SEQ ID NO: 1, the abscisic stress ripening protein comprises SEQ ID NO: 2, the actin 6 comprises SEQ ID NO: 3, the actin E comprises SEQ ID NO: 4, the biotin carboxylase precursor comprises SEQ ID NO: 5, the caffeic acid O-methyltransferase comprises SEQ ID NO: 6, the catalase 2 comprises SEQ ID NO: 7, the conserved-hypothetical-protein-of-Ricinus-communis ortholog comprises SEQ ID NO: 8, the fibrillin-like protein comprises SEQ ID NO: 9, the flavodoxin-like quinone reductase 1 comprises SEQ ID NO: 10, the fructose-bisphosphate aldolase comprises SEQ ID NO: 11, the glyceraldehyde 3-phosphate dehydrogenase comprises SEQ ID NO: 12, the H0825G02.11 ortholog comprises SEQ ID NO: 13, the large subunit of ribulose-1,5-bisphosphate carboxylase/oxygenase comprises SEQ ID NO: 14, the LealP comprises SEQ ID NO: 15, the methionine synthase protein comprises SEQ ID NO: 16, the mitochondrial peroxiredoxin comprises SEQ ID NO: 17, the Os02g0753300 ortholog comprises SEQ ID NO: 18, the Os05g0482700 ortholog comprises SEQ ID NO: 19, the Os12g0163700 ortholog comprises SEQ ID NO: 20, the OSJNBb0085F13.17 ortholog comprises SEQ ID NO: 21, the predicted-protein-of-Ostreococcus-lucimarinus-CCE9901 ortholog comprises SEQ ID NO: 22, the predicted-protein-of-Physcomitrella patens-subsp.-patens ortholog comprises SEQ ID NO: 23, the predicted-protein-of-Populus-trichocarpa ortholog comprises SEQ ID NO: 24, the hypothetical-protein-isoform-1-of-Vitis-vinifera ortholog comprises SEQ ID NO: 25, the nascent polypeptide associated complex alpha comprises SEQ ID NO: 26, the proline iminopeptidase comprises SEQ ID NO: 27, the protein transporter comprises SEQ ID NO: 28, the putative-NBS-LRR-disease-resistance-protein-homologue-of-Oryza-sativa-Japonica-Group ortholog comprises SEQ ID NO: 29, the Ran GTPase binding protein comprises SEQ ID NO: 30, the chloroplastic triosephosphate isomerase comprises SEQ ID NO: 31, the V-type proton ATPase catalytic subunit A comprises SEQ ID NO: 32, the regulator of ribonuclease activity A comprises SEQ ID NO: 33, the retroelement pol polyprotein-like ortholog comprises SEQ ID NO: 34, the ribosomal protein L10 comprises SEQ ID NO: 35, the short chain type dehydrogenase comprises SEQ ID NO: 36, the temperature-induced lipocalin comprises SEQ ID NO: 37, and the unknown-protein-of-Picea-sitchensis ortholog comprises SEQ ID NO: 38.

48. The method of claim 46 or 47, further comprising:
(i) determining the level of at least another of the proteins in the mesocarp tissue of the fruit of the test oil palm plant;
(ii) determining whether there is a difference between the level of the other protein in the mesocarp tissue of the test oil palm plant and the level of the other protein in the mesocarp tissue of the fruit of the reference oil palm plant; and
(iii) predicting the oil yield of the test oil palm plant based also on the difference with respect to the levels of the other protein.

49. A kit for obtaining a high-yielding oil palm plant comprising:
an antibody for detection of a protein selected from the group consisting of 5-methyltetrahydropteroyltriglutamate-homocysteine methyltransferase, abscisic stress ripening protein, actin 6, actin E, biotin carboxylase precursor, caffeic acid O-methyltransferase, catalase 2, conserved-hypothetical-protein-of-Ricinus-communis ortholog, fibrillin-like protein, flavodoxin-like quinone reductase 1, fructose-bisphosphate aldolase, glyceraldehyde 3-phosphate dehydrogenase, H0825G02.11 ortholog, large subunit of ribulose-1,5-bisphosphate carboxylase/oxygenase, LealP, methionine synthase protein, mitochondrial peroxiredoxin, Os02g0753300 ortholog, Os05g0482700 ortholog, Os12g0163700 ortholog, OSJNBb0085F13.17 ortholog, predicted-protein-of-Ostreococcus-lucimarinus-CCE9901 ortholog, predicted-protein-of-Physcomitrella patens-subsp.-patens ortholog, predicted-protein-of-Populus-trichocarpa ortholog, hypothetical-protein-isoform-1-of-Vitis-vinifera ortholog, nascent polypeptide associated complex alpha, proline iminopeptidase, protein transporter, putative-NBS-LRR-disease-resistance-protein-homologue-of-Oryza-sativa-Japonica-Group ortholog, Ran GTPase binding protein, chloroplastic triosephosphate isomerase, V-type proton ATPase catalytic subunit A, regulator of ribonuclease activity A, retroelement pal polyprotein-like ortholog, ribosomal protein L10, short chain type dehydrogenase, temperature-induced lipocalin, and unknown-protein-of-Picea-sitchensis ortholog; and
an extract of a mesocarp tissue of a fruit of a reference oil palm plant.

50. The kit of claim 49, wherein the 5-methyltetrahydropteroyltriglutamate-homocysteine methyltransferase comprises SEQ ID NO: 1, the abscisic stress ripening protein comprises SEQ ID NO: 2, the actin 6 comprises SEQ ID NO: 3, the actin E comprises SEQ ID NO: 4, the biotin carboxylase precursor comprises SEQ ID NO: 5, the caffeic acid O-methyltransferase comprises SEQ ID NO: 6, the catalase 2 comprises SEQ ID NO: 7, the conserved-hypothetical-protein-of-Ricinus-communis ortholog comprises SEQ ID NO: 8, the fibrillin-like protein comprises SEQ ID NO: 9, the flavodoxin-like quinone reductase 1 comprises SEQ ID NO: 10, the fructose-bisphosphate aldolase comprises SEQ ID NO: 11, the glyceraldehyde 3-phosphate dehydrogenase comprises SEQ ID NO: 12, the H0825G02.11 ortholog comprises SEQ ID NO: 13, the large subunit of ribulose-1,5-bisphosphate carboxylase/oxygenase comprises SEQ ID NO: 14, the LealP comprises SEQ ID NO: 15, the methionine synthase protein comprises SEQ ID NO: 16, the mitochondrial peroxiredoxin comprises SEQ ID NO: 17, the Os02g0753300 ortholog comprises SEQ ID NO: 18, the Os05g0482700 ortholog comprises SEQ ID NO: 19, the Os12g0163700 ortholog comprises SEQ ID NO: 20, the OSJNBb0085F13.17 ortholog comprises SEQ ID NO: 21, the predicted-protein-of-Ostreococcus-lucimarinus-CCE9901 ortholog comprises SEQ ID NO: 22, the predicted-protein-of-Physcomitrella patens-subsp.-patens ortholog comprises SEQ ID NO: 23, the predicted-protein-of-Populus-trichocarpa ortholog comprises SEQ ID NO: 24, the hypothetical-protein-isoform-1-of-Vitis-vinifera ortholog comprises SEQ ID NO: 25, the nascent polypeptide associated complex alpha comprises SEQ ID NO: 26, the proline iminopeptidase comprises SEQ ID NO: 27, the protein transporter comprises SEQ ID NO: 28, the putative-NBS-LRR-disease-resistance-protein-homologue-of-Oryza-sativa-Japonica-Group ortholog comprises SEQ ID NO: 29, the Ran GTPase binding protein comprises SEQ ID NO: 50, the chloroplastic triosephosphate isomerase comprises SEQ ID NO: 31, the V-type proton ATPase catalytic subunit A comprises SEQ ID NO: 32, the regulator of ribonuclease activity A comprises SEQ ID NO: 33, the retroelement pol polyprotein-like ortholog comprises SEQ ID NO: 34, the ribosomal protein L10 comprises SEQ ID NO: 35, the short chain type dehydrogenase comprises SEQ ID NO: 36, the temperature-induced lipocalin comprises SEQ ID NO: 37, and the unknown-protein-of-Picea-sitchensis ortholog comprises SEQ ID NO: 38.

51. The kit of claim 49 or 50, wherein the kit further comprises instructions indicating use of the antibody for determining whether there is a difference between the level of the protein in mesocarp tissue of a fruit of a parental oil palm plant and the level of the protein in the extract of the mesocarp tissue of the fruit of the reference oil palm plant.

52. The kit of claim 51, wherein the kit further comprises instructions indicating selection of progeny of the parental oil palm plant based on the difference to obtain the high-yielding oil palm plant.

53. The kit of claim 49 or 50, further comprising at least another antibody for detection of at least another of the proteins.


SEQUENCE LISTING (45 sequences)

SEQ ID NO: 1 - protein, 210 aa, Elaeis guineensis
MASHVVGYPRMGPKRELKFALESFWDGKSSAADLQKVSQDLRCSIWKQMSDAGVKYIPSNTFSYYDQVLDATAMLGAVPQRYGWSGGEIGFDTYFSMARGNASVPAMEMTKWFDTNYHYIVPELAPDTKFTYASHKAVSEYKEAKALGIDTVPVLIGPVTYLLLSKPAKGVEKAFPLLSLLGNVLPVYKEVIAELKAAGASWVQFDEPHL

SEQ ID NO: 2 - protein, 68 aa, Elaeis guineensis
  NON_TER (68)..(68)
MEHLGEMGAVAAGAFALYEKHEAKKHPEHAHRHKIEEEIAAAVAVGSGGYAFHEHHEKKAAKKEAEES

SEQ ID NO: 3 - protein, 377 aa, Elaeis guineensis
MADAEDIQPLVCDNGTGMVKAGFAGDDAPRAVFPSIVGRPRHTGVMVGMGQKDAYVGDEAQSKRGILTLKYPIEHGIVSNWDDMEKIWHHTFYNELRVAPEEHPVLLTEAPLNPKANREKMTQIMFETFNVPAMYVAIQAVLSLYASGRTTGIVLDSGDGVSHTVPIYEGYALPHAILRLDLAGRDLTDALMKILTERGYSFTTTAEREIVRDMKEKLAYVALDYEQELETAKSSSSVEKSYELPDGQVITIGAERFRCPEVLFQPSLIGMEAAGIHETTYNSIMKCDVDIRKDLYGNIVLSGGSTMFPGIADRMSKEITALAPSSMKIKVVAPPERKYSVWIGGSILASLSTFQQMWISKGEYDESGPSIVHRKCF

SEQ ID NO: 4 - protein, 377 aa, Elaeis guineensis
MAEAEDIQPLVCDNGTGMVKAGFAGDDAPRAVFPSIVGRPRHTGVMVGMGQKDAYVGDEAQSKRGILTLKYPIEHGIVTNWDDMEKIWHHTFYNELRVAPEEHPVLLTEAPLNPKANREKMTQIMFETFNVPAMYVAIQAVLSLYASGRTTGIVLDSGDGVTHTVPIYEGFALPHAILRLDLAGRDLTDALMKILTERGYSFTTTAEREIVRDIKEKLGYVALDYEQELETAKSSSSVEKSYELPDGQVITIGAERFRCPEVLFQPSLIGMEAAGIHETTYNSIMKCDVDIRKDLYGNIVLSGGSTMFPGIADRMSKEISALAPSSMKIKVVAPPERKYSVWIGGSILASLSTFQQMWISKEEYDECGPAIVHRKCF

SEQ ID NO: 5 - protein, 535 aa, Elaeis guineensis
MDSMIACKSGCLPPGLFIRPTRGIRSSQCSFMVGNDPKFCKISFPRKRTACAIRKSKKNGGALNATCYDEKILVANRGEIAVRVIRTAHEMGIPCVAVHSTIDQDALHVRLADEAVCIGEAPSSQSYLFIPNVLSAAVSRGCTMLHPGYGFLAENAGFVDICKEHGINFIGPNPDSIRVMGDKSTARETMKKAGVPTVPGSDGLLQSTEEAVKLAHEIGFPVMIKATAGGGGRGMRLAYEPEQFVKLLQQAKSEAAAAFGNDGVYLEKYIQNPRHIEFQVLADKYGNVVHFGERDCSIQRRNQKLLEEAPSPALTPELRKAMGDAAVAAAASIGYIGVGTVEFLLDERGSFYFMEMNTRIQVEHPVTEMISSTDLIEEQIRVALGERLTYKQEDIVLRGHSIECRINAEDAFKGFRPGPGKITSYLPSGGPFVRMDSHVYPGYVVPPSYDSLLGKLIVWAPTREKAIERMKRALDDTIITGIPTTIEYHKLILDIEDFRNGKVDTAFIPKHEKDLTAPHKLVLSTSEKELAGVGA

SEQ ID NO: 6 - protein, 367 aa, Elaeis guineensis
MGSIEKQGMPAPSVENQAGDEASCMYALQLASSSILPMTLKAAIELGVLEIIAKAGSVGKLSPAQVAAQLPTENPMAATMLDRMLRLLASYNILTCSVEVDADGKPLRRYGPAPVCKWLTKNEDGVSMAALTLMNQDKVLMESWYYLKDAVLDGGIPFNKAYGMTAFEYHGTDPRFNKVFNEGMKNHSIIITKKLLEFYKGFEDVNVLVDVGGGIGATLHMITSKYPHIKGINFDLPHVISEAPPFPGVEHIGGDMFASVPSGDAILMKWIMHDWSDEHCAKILENCYKALPNNGKVILCECILPVAPEPVPSAQGVFHVDMIMLAHNPGGKERTEKEYQDLAKGAGFTGFKAQYCFSTAWVLEFTK

SEQ ID NO: 7 - protein, 326 aa, Elaeis guineensis
  NON_TER (326)..(326)
MDPYKFRPSSANNSPFWTTNSGAPVWNNNSSLTVGNRGPILLEDYHVIEKLAQFDRERIPERVVHARGASAKGFFEVTHDVSHLTCADFLHAPGVQTPVIVRFSTVIHERGSPETLRDPRGFAVKFYTREGNFDLVGNNFPVFFVRDGIKFPDMVHALKPNPKSHIQENWRIVDFFSHHPESLHMFTFLFDDVGVPADYRHMDGSGVNTYTLINKEGKARYVKFHWRPTCGVKCLLEDEAVIVGGNNHSHATKDLYDSIAAGNYPEWKLFIQTIDPDHEDRFDFDPLDVTKTWPEDILPLQPVGRMVLNKNIDNFFAENEQLAFCP

SEQ ID NO: 8 - protein, 170 aa, Elaeis guineensis
MAAIANSFVSIRDLRPPALSGCPLKPSPQSLVSCSPMNLSLSTIRWRRAKIATYKSRITVQAAYSDGGRPNSASIFIGGFVLGGIIVGTLGCVYAPQISKILAGADRKDLMRKLPKFIYDEEKALERTRKILADKIEQLNSAIDDVSSQLRADDGPNGVAVASDEVEAAI

SEQ ID NO: 9 - protein, 328 aa, Elaeis guineensis
MAGVASWKPMALKAPPAAPPLRYHSSIHPIPIGAPASVFQKAPILRSAMGLPLAAAPIRPRAIADDEEWGGELGEEEGAAAGGLAVAEQEEEEVKVEPIEVGELKRKLMDLLYGTDRGLQASSETRAEIVEVINQLEVRNPTPAPTEALTLLNGKWILAYTSFSPLFPLLGSARLPELVKVEEISQTIDSENFTVQNSVQFAVPLATTSVTTNAKFEVRSPKRVQIKFEEGIIGTPQLTDSIVLPEKVEFLGQNIDLSPFKSVITSVQNAASSVARTISERPPWKISINNDNAQSWLLTTYLDEELKISRADGGGIFLLIKEGSSLLN

SEQ ID NO: 10 - protein, 203 aa, Elaeis guineensis
MATKVYIVYYSMYGHVEKLAEEIKRGASTVGGVEAKMWQVPETLPEEVLGKMGAPPKSDVPIITPNELAEADGIIFGFPTRFGMMAAQFKAFLDATGGLWRAQQLAGKPAGIFYSTGSQGGGQETTPLTAITQLVHHGMIFVPIGYTFGAGMFEMEKVKGGSPYGAGTYAGDGSRFPSELELEQAFHQGKYFAGIAKKFKSSS

SEQ ID NO: 11 - protein, 358 aa, Elaeis guineensis
MSAYCGKYHDELIKNAAYIGTPGKGILAADESTGTIGKRLASINVENVEENRRALRELLFCTPGALQYLSGVILFEETLYQKTKDGKPFVEVLKEGGVLPGIKVDKGTIELAGTNGETTTQGHDDLAKRCQKYYEAGARFAKWRAVLKIGPTEPSQLAIDLNAHGLARYAIICQENGLVPIVEPEILVDGPHDIKRCADVTERVLAACYKALNDHHVLLEGTLLKPNMVTPGSESAKVAPEVVAEYAVRALQRTVPAAVPAIVFLSGGQSEEEATLNLNAMNKLGGKKPWSLSFSFGRALQQSTLKAWAGKVENVEKARAAFLARCKANSEATLGKYEGDAAGGEGVSESLHVKDYKY

SEQ ID NO: 12 - protein, 206 aa, Elaeis guineensis
MFVVGVNEKEYKSDINVVSNASCTTNCLAPLAKVIHDKFGIVEGLMTTVHSITATQKTVDGPSSKDWRGGRAASFNIIPSSTGAAKAVGKVLPALNGKLTGMAFRVPTVDVSVVDLTVRLEKSATYDQIKDAIKEESEGKMKGILGYVDEDLVSTDFLGDSRSSIFDAKAGIALNGNFVKLVSWYDNEWGYSSRVVDLIRHIHGTQ

SEQ ID NO: 13 - protein, 116 aa, Elaeis guineensis
  NON_CONS (14)..(15)
  NON_CONS (20)..(21)
  NON_CONS (30)..(31)
  NON_CONS (38)..(39)
  NON_CONS (49)..(50)
  NON_CONS (70)..(71)
  NON_CONS (78)..(79)
  NON_CONS (108)..(109)
  NON_TER (116)..(116)
MDLGKSASTSATLKFYYELRYVPFQCPSSRRIQPLQARELAEAHEDLTKLSCPELDLREILDQGAASDVRFEVAELEREKAVETLRGREVWFSSYLQSCCTSMARVCRSMSPLAEK

SEQ ID NO: 14 - protein, 56 aa, Elaeis guineensis
  NON_TER (1)..(1)
  NON_CONS (23)..(24)
  NON_CONS (31)..(32)
  NON_TER (56)..(56)
DYRLTYYTPDYVVRDTDVLAAFRALRLEDLRSQAETGEVKGHYLNVTAGTAEEMLK

SEQ ID NO: 15 - protein, 81 aa, Elaeis guineensis
  NON_CONS (10)..(11)
  NON_CONS (25)..(26)
  NON_CONS (34)..(35)
  NON_CONS (45)..(46)
  NON_TER (81)..(81)
MASHQDTSYKDQTGSYVSDKASAARSCETGQAAKEKASQMGESAKTGGVISSAAEQVKGMAQGATEAVKNTFGMGGGNEEK

SEQ ID NO: 16 - protein, 413 aa, Elaeis guineensis
  NON_TER (413)..(413)
MGPKRELKFALESFWDGKSSADDLQKVAADLRCSIWKQMADAGIKYIPSNTFSYYDQVLDTTAMLGAVPQRYGWSGAEIGFDTYFSMARGNALVPAMEMTKWFDTNYHYIVPELAPDTKFTYASHKAVSEYKEAKALGIDTVPVLIGPVTYLLLSKPAKGVEKSFATLSLLGNVLPIYQEVIMELKAAGASWIQFDEPTLVMDLDAHQLEAFTKAYSELESSFSGLNVLVETYFADVPAEAYKTITTLNGVSGFGFDLVRGTNTLDLIKSAGFPSGKYLFAGVVDGRNIWANDLASSLSSLGVLEAIVGKDNLVVSTSCSLMHTAVDLVNETKLDGEIKSWLAFAAQKVVEVNALAKALAGHKDEAFFSANAAAQASRKSSPRVTNEEVQTAAAALKGSDHCRATNVSARLDA

SEQ ID NO: 17 - protein, 197 aa, Elaeis guineensis
MASALMRARRSAALPSAAVMALRRAFAAASVGCDIVSAAPGVALQKARSWDEGVSSKFSTTPLKDIFLGKQVVVFGLPGAFTGVCSAQHVPSYKNNIDKFKAKGIDSVICVAVNDPYVLNGWAENLQAKEAIDFYGDFDGSFHKSLELDLDLSAALLGHRSQRWSAYVVDGKIKVLNVEKVPSEFKVSGGEVILGQF

SEQ ID NO: 18 - protein, 195 aa, Elaeis guineensis
MMKGTKKAIFLFLLLSSLVIISHCVTPLTIKPVNRDDDEYQCVYTVYIRTGSIWKGGTDSVISLVLAGSDGWGVAITDIESWGGIMEAGHNYFERGNLDIFSGRGPCLSTPPCWMNLTSDGSGSHHGWYCNYVEVTTTGPHMGCAQQLFTVEQWLATDAAPYQLYATRDSCTGRGNAEEEPKIGDRIGRHVTHVA

SEQ ID NO: 19 - protein, 559 aa, Elaeis guineensis
MGSSEFSWKLADHPKLPKGKTVAVVVLDGWGEANPDKYNCIHVAETPAMDSLKKGAPDRWRLVRAHGTAVGLPTEDDMGNSEVGHNALGAGRIYAQGAKLVDLALASGKIYEGEGFKYIKECFDQGTLHLIGLLSDGGVHSRLDQLQLLLKGASENGAKRIRVHVLTDGRDVLDGSSVGFVETLENDLLKLREKGVDAQIASGGGRMYVTMDRYENDWGVVKRGWDAQVLGEAPHKFQSAVEAVKKLREVPKANDQYLAPFVIVDESGKAVGPIVDGDAVVTFNFRADRMVMLAKALEYEDFDKFDRVRFPKIQYAGMLQYDGELKLPSHYLVSPPEIERTSGEYLVHNGIRTFACSETVKFGHVTFFWNGNRSGYFDPSMEEYVEIPSDAGITFNVKPKMKAVEIAEKARDAILSRKFDQVRVNLPNGDMVGHTGDIEATVVACKAADDAVKMILDAIEQVGGIYVVTADHGNAEDMVKRNKSGQPQLDKNGDIQILTSHTLQPVPVAIGGPGLAPGVGFRKDVPNGGLANIAATVMNLHGFEAPSDYEPTLIEVVDN

SEQ ID NO: 20 - protein, 377 aa, Elaeis guineensis
MADAEDIQPLVCDNGTGMVKAGFAGDDAPRAVFPSIVGRPRHTGVMVGMGQKDAYVGDEAQSKRGILTLKYPIEHGIVSNWDDMEKIWHHTFYNELRVAPEEHPILLTEAPLNPKANREKMTQIMFETFDVPAMYVAIQAVLSLYASGRTTGIVLDSGDGVSHTVPIYEGYALPHAILRLDLAGRDLTDALMKILTERGYSFTTTAEREIVRDIKEKLAYVALDYEQELETAKSSSAVEKSYELPDGQVITIGAERFRCPEVLFQPSLIGMESPGIHETTYNSIMKCDVDIRKDLYGNIVLSGGSTMFPGIGDRMSKEITALAPSSMKIKVVAPPERKYSVWIGGSILASLSTFQQMWITKAEYDESGPAIVHRKCF

SEQ ID NO: 21 - protein, 702 aa, Elaeis guineensis
MADIQMGEAETFAFQAEINQLLSLIINTFYSNKEIFLRELISNSSDALDKIRYEGLTDKSKLDAQPELFIRLVPDKANKTLSIIDSGIGMTKADLVNNLGTIARSGTKEFMEALQAGADVSMIGQFGVGFYSAYLVAEKVVVTTKHNDDEQYIWESQAGGSFTVTRDVSGEQLGRGTKITLYLKEDQLDYLEERRLKDLVKKHSEFISYPIYLWTEKTTEKEISDDEDEETKKEEEGDVENIDEEKETKSKKKKVKEVLHEWSLVNKQKPIWLRKPEEITKEEYASFYKSLTNDWEDHLAVKHFSVEGQLEFKAILFVPKRAPFDLFDTRKKMNNIKLYVRRVFIMDNCEELIPEYLGFVKGVVDSDDLPLNISREMLQQNKILKVIRKNLVKKCIELFSEIAENKEDYNKFYEAFSKNLKLGIHEDSQNRAKLADLLRYHSTKSGDEMTSLKDYVTRMKEDQKDIYYITGESKKAVENSPFLEKLKKKGYEVLFMVDAIDEYAVGQLKEYDGKKLVSATKEGLKIDESEDEKKRKEEKKAAFESLCKVMKDILGDKVEKVVVSDRIVDSPCCLVTGEYGWTANMERIMKAQALKDNSMGSYMSSKKTMEINPENGIMEEMRKRAEADKNDKSVKDLVLLLFETAILTSGFSLDDPNTFAGRIHRMLKLGLSIDEDEAAGDDTDMPALEEDGNEESKMEEVD

SEQ ID NO: 22 - protein, 26 aa, Elaeis guineensis
  NON_TER (1)..(1)
  NON_CONS (6)..(7)
  NON_TER (26)..(26)
MKWCERVYLTIELPDAKDAEVTIEAR

SEQ ID NO: 23 - protein, 29 aa, Elaeis guineensis
  NON_TER (1)..(1)
  NON_CONS (21)..(22)
  NON_TER (29)..(29)
YIANKFEGQGTPIYGSTPVEKVLDIYEAR

SEQ ID NO: 24 - protein, 707 aa, Elaeis guineensis
MASSSAQIHVLGSAAAFPSSRKPSARISSSKSLFFGNRHSNGINGAFLSIRRGAGRGNRGYGGPLRVVSEKVVGIDLGTTNSAVAAMEGGKPTIVTNAEGQRTTPSVVAYTKNGDRLVGQIAKRQAVVNPENTFFSVKRFIGRKMSEVDEESKQVSYRVVKDENGNVKLECPAIGKQFAAEEISAQVLRKLVDDASKFLNDKVTKAVVTVPAYFNDSQRTATKDAGRIAGLDVLRIINEPTAASLAYGFEKKNNETILVFDLGGGTFDVSVLEVGDGVFEVLSTSGDTHLGGDDFDKRVVDWLAGNFKRDEGIDLLKDKQALQRLTETAEKAKMELSSLTQTNISLPFITATADGPKHIETTITRAKFEELCSDLLDRLRTPVDNALKDAKLSFKDIDEVILVGGSTRIPAVQELVKKMTGKEPNVTVNPDEVVALGAAVQAGVLAGDVSDIVLLDVTPLSLGLETLGGVMTKIIPRNTTLPTSKSEVFSTAADGQTSVEINVLQGEREFVRDNKSLGSFRLDGIPPAPRGVPQIEVKFDIDANGILSVTAVDKGTGKKQDITITGASTLPGDEVDRMVKEAERYAKEDKEKRDAIDTKNQSESVVYQTEKQLKELGDKVPAAVKEKVEAKLKELKDAIAGGSTQSMKDAMAALNQEVMQLGQSLYNQPGAAGAGPAPGADAGSAGSTGKGPDDGGDVIDADFTDSK

SEQ ID NO: 25 - protein, 205 aa, Elaeis guineensis
MQGALQAIRAQGNVLKCAVLRHVRIMNPAMLPTAISRFESTSPAQLEEHGFESTTISDVLKAKGKGADGSWLWCTTDDTVYDAVKSMTQHNVGALVVVKPGADKAIAGIITERDYLRKIIVQGRSSKSTKVGDIMTEENKLITVTPDTKVLQAMQLMTDNRIRHIPVIDAKGMMGMVSIGDVVRAVVTEHREELDRLNAYIQGGY

SEQ ID NO: 26 - protein, 201 aa, Elaeis guineensis
MTAQTQEELLAAHLEQQKIDYDEPVVEDEDDDEDDEDDDDKDEDDAEGQAGDASGRSKQSRSEKKSRKAMLKLGMKPIPGVSRVTVKKSKNILFVISKPDVFKSPTSDTYVIFGEAKIEDLSSQLQTQAAEQFKAPDLSHVISKPETSAMAQDDEEVDETGVEPKDIELVMTQAGVSRSKAVKALKAADGDIVTAIMELTN

SEQ ID NO: 27 - protein, 312 aa, Elaeis guineensis
METSKESQELNRSLYPHIEPYSSGFLKVSDIHTLYWEQSGNPNGHPVVFLHGGPGAGTSASNRRFFDPEFYRIILFDQRGSGKSTPHACLEDNTTWDLVNDIEKLREHLEIPEWQVTGIILRGIFLLRKKELDWFYEGGAAAIFPDAWEPFRDFIPENERNNFIVAYHKRLNSDDNHIQVTAAKIWTTWELMTAHLIQNEENIKRGEDDDFSLAFARIENHYFVNKGFFPSDSYLLDNVDKIRHISTVIVQGRYDVCCPMMSAWDLHKAWPEAELKVVPDAGHSANEIGIAAELVAANEKFKNILKSGGFKM

SEQ ID NO: 28 - protein, 289 aa, Elaeis guineensis
MASSGPDHLFNLRNSFYLGAYQDAINKSDIPNLSGDDAVERDSIVYRSYIALGSYQLVINEIDSSAPTALQAVKLLALYLAGDKESAIASLQEWLNDAAIGNNHVLRLIAGIIYMHEQNYVEALKHTNSGGTMELHALNVQIYLKMHRSDYAEKQLKIMLQIDEDHTLTQLANAWVNLAVGGSKIQEAYLIYQDFSEKNQMTPLILNGKAVCCLHMGRFDEAESLLLDALNKDAKDAETLANLVVCSLHLGKSAARYLNQLKLSHPDHMLVQRASSAEDSFERAVQSIA

SEQ ID NO: 29 - protein, 224 aa, Elaeis guineensis
  NON_TER (1)..(1)
  NON_CONS (39)..(40)
  NON_CONS (48)..(49)
  NON_CONS (56)..(57)
  NON_CONS (85)..(86)
  NON_CONS (106)..(107)
  NON_CONS (126)..(127)
  NON_CONS (143)..(144)
  NON_CONS (155)..(156)
  NON_CONS (168)..(169)
  NON_CONS (173)..(174)
  NON_CONS (192)..(193)
  NON_CONS (199)..(200)
  NON_CONS (210)..(211)
  NON_TER (224)..(224)
ERQEIDRSAMVGALASASTGVMNSVIAKLSKLLEDEYAKAHVVEESKRLRDTLQDKYLVVIDDVWATEAWETIKLALLSNNCDSRCSYHGGYVYHMEPLSFVDSKRVLAAIGSALAKDPNAGNMRRSLIQPVDVQYGKPVACRENVIMATNLSLKMQSLEELQTFDIFTYSSRWMNMLACLTELDLTLCSTKLQHLMIKCLNGASDLGIRSVNYEIEVNLGEVR

SEQ ID NO: 30 - protein, 176 aa, Elaeis guineensis
  NON_TER (1)..(1)
  NON_CONS (46)..(47)
  NON_CONS (66)..(67)
  NON_CONS (82)..(83)
  NON_CONS (103)..(104)
  NON_CONS (120)..(121)
  NON_CONS (130)..(131)
  NON_CONS (151)..(152)
  NON_CONS (161)..(162)
  NON_TER (176)..(176)
FQKDMSFRFVTPAYGCTQIGKNDSLKDVLMWGEGVEGGNIGGMVQRVNMDVGMPKVVESLDDVHIKFNAVGGKLFTWGDGDKSISVTDQSSCSGCRMPFGLTRHNCYNCGLLFCHSCSSKMSKPGTHGSKDQGEHQHHVETVSSLSAGLPRAMSEKISAGRGGVELSVSQNTPAYK

SEQ ID NO: 31 - protein, 307 aa, Elaeis guineensis
MAAASSLVPQFSGLAVARERAPSLSSILFFHDASSQRRPFSTRRAPRGVVAMAGTGKFFVGGNWKCNGMKDSISKLVADLNDAKMENDVDVAVAPPFIYIDQVNNSLTDRIDISAQNCWVGKGGAFTGEISAEQLIDMGCKWVILGHSERRHIIGEDDQFIGKKAAYALSQNLKVIACIGEKLEEREAGKTFEVCFQQMQAFADSITSWENVVIAYEPVWAIGTGKVATPEQAQEVHVAVRDWLKKNISAEVASSTRIIYGGSVNGSNCSELAKQEDIDGFLVGGASLKGPEFATIVNSVTSKKVAV

SEQ ID NO: 32 - protein, 621 aa, Elaeis guineensis
MAYGDRLTTFEDSEKESEYGYVRKVSGPVVVADGMGGAAMYELVRVGHDNLIGEIIRLEGDSATIQVYEETAGLMVNDPVLRTHKPLSVELGPGILGNIFDGIQRPLKTIALKSGDVYIPRGVSVPALDKDTLWEFEPKKLAAGDLLTGGDLYATVFENTLMQHHVALPPGSMGKITYIAPAGQYSLKDTVLELEFQGVKKQFTMLQTWPVRTPRPVAAKLAADTPLLTGQRVLDALFPSVLGGTCAIPGAFGCGKTVISQALSKYSNSDTVVYVGCGERGNEMAEVLMDFPQLTMTLPDGREESVMKRTTLVANTSNMPVAAREASIYTGITIAEYFRDMGYNVSMMADSTSRWAEALREISGRLAEMPADSGYPAYLAARLASFYERAGKVKCLGGPDRTGSVTIVGAVSPPGGDFSDPVTSATLGIVQVFWGLDKKLAQRKHFPSVNWLISYSKYSKALESFYEKFDPDFIDIRTKAREVLQREDDLNEIVQLVGKDALAESDKITLETAKLLREDYLAQNAFTPYDKFCPFYKSVWMMRNIIHFNALANQAVERGAGSDGQKITYSVIKHRLGDLFYRLVSQKFEDPAEGEEVLVAKFKKLYDDLSAGFRNLEDETR

SEQ ID NO: 33 - protein, 168 aa, Elaeis guineensis
MAALPLATAEVCDANAHLIQNGELRALQPIFQIYGRRQVFAGPIVTLKVFEDNVLVREFLEEKGNGRVLVVDGGGSMRCAILGGNPVQQAQNNGWAGIVVNGCIRDVDEINGCDIGVRALASHPMKANKKGFGEKHVPVNIAGTRICDGEWLYADSDGILVSRMELTV

SEQ ID NO: 34 - protein, 192 aa, Elaeis guineensis
  NON_CONS (18)..(19)
  NON_CONS (39)..(40)
  NON_CONS (57)..(58)
  NON_CONS (75)..(76)
  NON_CONS (86)..(87)
  NON_CONS (126)..(127)
  NON_CONS (134)..(135)
  NON_CONS (148)..(149)
  NON_CONS (166)..(167)
  NON_CONS (179)..(180)
  NON_CONS (192)..(192)
MTIDSELLTNISHRDVARMKADLATCKQEGMTMEGYYGKICASHHMTGNLELLSDMRTPSYSGARYFLTIVDDYSSDNGTEFLCMRFQSYLPIQFWGECILSAAYLINRTPSMLLQGKSPYEMLYKLFDLEEQKVNQTFSIVNLPPGKEEVYMKLPQGFQCDDPSKAMAFLTQELMWLKQLADILTKALGEK

SEQ ID NO: 35 - protein, 321 aa, Elaeis guineensis
MTVKLSKSEKKIEYDKKLCRLLDEYSQVLIAAADNVGSNQLQNIRKGLRGDSVVLMGKNTLIRRCIKIHAEKTGNKDYLNLLPLLVGNVGLIFTKGDLKEVREEVAKYKVGAPARVGLVAPIDVVVPPGNTGLDPSQTSFFQVLNIPTKINKGTVEIITPVELIRKGDKVGSSEAALLAKLGIRPFSYGLVIQAVYDNGSVFSPEVLDLTEDDLVEKFAAGVSMVTSLSLALSYPTLVAAPHMFINAYKNVLAVAIATEYTFEQAEKVKEYLKDPSKFAVAVPVEAAAAAAAAPAAAAKEEEKKEEPAEESDEDMGFSLFD

SEQ ID NO: 36 - protein, 176 aa, Elaeis guineensis
MIKTAVDAWGTVDILVNNAGITRDTLLMRMKKPQWQEVIDLNLTGVFLCTQAAAKLMMKKKKGKIINMTSVIGLTGNIGQANYSAAKAGVIGFTKTVAREYASRNINVNAVAPGFIASDMTAKLGADIETKFLQTIPLGRFGQPEEVAGLVEFLALNPAANYITGQVFTIDGGLVM

SEQ ID NO: 37 - protein, 188 aa, Elaeis guineensis
MATTSTKEMTVVKGLDVERYMGRWYEIASFPSFFQPKNGENTRATYTLNSDGTVHVLNETWSDGKRDAIEGTAYKANPNSDEAKLKVKFYVPPFFPIIPVTGDYWVLYIDDDYQYALIGQPSRKYLWILCRQTHMDDEVYNLLLEKAKEEGYDVKKLHKTPQADPPPEAEAGPKDTKGIWWIKSLFGK

SEQ ID NO: 38 - protein, 319 aa, Elaeis guineensis
MAASIAFNPHVIPARSAAPRRVAPLQTLSAAPRLAARSLCFKSSLRSDGTAAFSGVRTQAAVVEQASIEQAQNVEAPVAIVTGGSRGIGKAIALALGKAGCKVLINYATSTEEAEEVSKEIEASGGQAIIFFGDISKEDDVESMIKIAADAWGTVDILVNNAGITRDTLLMRMKKSQWQEVIDVNLTGVFLCTQAAAKLMMKKKKGKIINIASVVGLTGNAGQANYSASKAGVIGFTKTVAREYASRNINVNAVAPGFIASPMTAQLGEDVEKKILQTIPLGRYGQPEEVAGLVEFLALNPAADYITGQVFTIDGGMVM

SEQ ID NO: 39 - protein, 155 aa, Elaeis guineensis
MSLIPRGWGFGSSIFDPWDSFEGFPFNSSLSSFPRPSFPSESSAFANARIDWKETPEAHVFKADLPGLKKEEVKVEVEEGNVLQISGERSREQEEKTDTWHRVERSSGKFLRRFRLPENAKMDRVKAAMENGVLTVTVPKEEVKKLEVRSIEISG

SEQ ID NO: 40 - protein, 667 aa, Elaeis guineensis
MEFYTTCLPRSSMSYCAHPRLSPPVPRSKQFLSFRIKNAASTSSVPAPPATTPSSSRNGNITTKSLTEKLSSAMDQLDIERGVCIPFRKYTPETVRNKVLESRGAILSLIGRGVEIVWNLGLYWSALTYDCLVGRDEEVVPYRARQLRNLLCNLGPSFIKAGQVLANRPDIIREDYMNELCILQDDVPPFPNQEAFSIIEEELGQPLEKVFSRISSETIAAASLGQVYRATLRETREDVAIKVQRPGIEPIIYRDLFLFRTLASFLNGISLQKLGCNAELIVDEFGEKLLEELDYTLEARNIEDFLENFKNDPTVKIPRVYKQLSGRRVLVMEWIDGIRCTDPEAVKEAGIDVNGFLTVGVSAALRQLLEFGLFHGDPHPGNIFAMRDGRIAYVDFGNVAVLSQQNKQILIDAVVHAVNEDYAEMANDFTRLGFLASGTDVSPIIPALEAIWQNSAGKGLADFNFRSVTGKFNQLVYNYPIRIPERFSLVIRSLLTQEGICFTLKPDFKFLEVAYPYIAKRLLTDPNPALRERLIQVLFKDGLFQWKRLENLIVLAKENVAKMNQNPALQVTNTQNSRNWQVEKKLDLTETIKDGARMFLIDAGIRRQLIMALTEDSKLHIQELVDVYRLVEDDVDIPAVAFEVLQDLPSVTRDLMLSWSEAVLSDR

SEQ ID NO: 41 - protein, 170 aa, Elaeis guineensis
MAGNSSQATQTVHDFTVKDARGNDVNLGTYKGKVLLIVNVASQCGLTNSNYTELTALYEKYKDKGLEILAFPCNQFGAQEPGSNEQILEFACTRFKAEYPIFDKVDVNGDNAAPIYKFLKSSKGGLFGESIKWNFSKFLVDKEGHVVDRYAPTTSPLSIEKDVKKLLGLN

SEQ ID NO: 42 - protein, 37 aa, Elaeis guineensis
  NON_TER (1)..(1)
  NON_CONS (7)..(8)
  NON_CONS (19)..(20)
  NON_CONS (28)..(29)
  NON_TER (37)..(37)
GIEYEYKSSLLLEMNPVHKFWADFVDKKGEEQETAKK

SEQ ID NO: 43 - protein, 216 aa, Elaeis guineensis
MGVKVYGPTMSTCTARVLLCLEEVGAEYELVPINLSTGEHKQPAHLARNPFGQVPAFEDGALMLHESRAIARYVSRKYKSSGADLLKEGGLEESAMVDVWLEVESHQFDPAIGPIFFQSFIVPMIGGVPDQTVINTNLEKLCKVLDIYEARLSKTKYLAGDFFSLADLSHVPLLYYFMGSPHASVVNSRPHVKAWWEAVSSRPACKKVTSAMPGSA

SEQ ID NO: 44 - protein, 813 aa, Elaeis guineensis
MAQILLHGTLHVTIFEANSLSNPNRASGGAPKFIRQLVEGIEDTIGLGKGSSKLYATIDLEKARVGRTRLITKEPVNPRWYESFHIYCAHMSANVIFTVKFDNPIGASLIGRAYLPVTEILNGEEVDRWIEICDEDRNPLDGGARIHVKVQYFDISKDRNWARGIRSAKYPGVPYTFFSQRQGCKVTLYQDAHVPDNFIPKIPLADGKYYEPHRCWEDIFDAISNAQHLIYITGWSVYTEITLVRDAKRQKPGGDVTLGELLKRKASEGVRVLMLVWDDRTSVGLLKKDGLMATHDEETANYFQDTDVHCVLCPRNPDDGGSFVQDLQISTMFTHHQKIVVVDHEMPNKSSQQRRIVSFVGGIDLCDGRYDTQFHSLFRTLDTAHHDDFHQPNFADASIKKGGPREPWHDIHSRLEGPIAWDVLYNFEQRWRKQGGKDLLVQLRDLADIVIPPSPVMFPEDRETWNVQLFRSIDGGAAFGFPETPEDAARAGLVSGKDNIIDRSIQDAYINAIRRAKNFIYIENQYFLGSSFGWRADDIKPEEVGALHLIPKELSLKIVSKIEAGERFTVYVVVPMWPEGVPESGSVQAILDWQRRTMEMMYTDIILALQAKGIEANPKDYLTFFCLGNREVKKGGEYEPEEQPEADTDYSRAQQARRFMIYVHTKMMIVDDEYIIIGSANINQRSMDGARDSEIAMGAYQPFYLSTRGLARGRIHGFRMALWYEHLGMLDDAFLHPESVECVQKVNRIADKYWDLYSSDNLDRDLPGHLLSYPIGVSSDGVITELPGMEFFPDTRARVLGTKADYLPPILTT

SEQ ID NO: 45 - protein, 92 aa, Elaeis guineensis
  NON_TER (1)..(1)
  NON_CONS (18)..(19)
  NON_CONS (25)..(26)
  NON_CONS (37)..(38)
  NON_CONS (58)..(59)
  NON_CONS (71)..(72)
  NON_CONS (83)..(84)
  NON_TER (92)..(92)
LSLDAKSEILPFLPCIFNRFDSVTKEITDLGMESFARLSCGSCTFGAKGINAMLEHCKLQVTDIGLFGISKRIGDQGLMSVAKNLVACTLRR